(19) United States
(12) Patent Application Publication
(10) Pub. No.: US 2022/0370638 A1
(43) Pub. Date: Nov. 24, 2022

(54) COMPOSITIONS AND METHODS FOR TREATMENT OF MAPLE SYRUP URINE DISEASE

(71) Applicant: The Trustees of the University of Pennsylvania, Philadelphia, PA (US)

(72) Inventors: Jenny Agnes Sidrane, Phoenixville, PA (US); James M. Wilson, Philadelphia, PA (US)

(21) Appl. No.: 17/620,490

(22) PCT Filed: Jun. 20, 2020
(86) PCT No.: PCT/US2020/038841

§ 371 Date: Dec. 17, 2021

Related U.S. Application Data
(60) Provisional application No. 63/016,240, filed on Apr. 27, 2020.
(60) Provisional application No. 62/864,263, filed on Jun. 20, 2019.

(51) Int. Cl. A61K 48/00; A61P 3/00; C12N 15/86; C12N 9/02

(52) U.S. Cl. CPC A61K 48/0058; A61P 3/00; C12N 15/86; C12N 9/0008; C12Y 102/04004; A61K 48/0075; C12N 2750/14143

(57) ABSTRACT

Provided herein are combination therapies involving co-expression of an E2 subunit of a branched-chain alpha-keto acid dehydrogenase (BCKDH) from a skeletal muscle-targeted rAAV.hDBT vector and a liver-targeted rAAV.hDBT vector. Also provided herein are combination therapies wherein an E1a and/or an E1b subunit of the BCKDH complex is expressed from muscle and/or liver following rAAV-mediated delivery targeted to these tissues. Further provided is a pharmaceutical composition comprising a rAAV as described herein in a formulation buffer, and a method of treating a human subject diagnosed with MSUD.

### BACKGROUND OF THE INVENTION

Maple syrup urine disease (MSUD) is a rare, inherited metabolic disorder characterized by the dysfunction of the mitochondrial enzyme complex branched chain alpha-keto acid dehydrogenase (BCKDH). MSUD has an estimated incidence of 1 in 185,000 live births in the generation population, [Hinton C F, et al. Developing a public health-tracking system for follow-up of newborn screening metabolic conditions: a four-state pilot project structure and initial findings. Genet Med. 2014; 16(6):484-90; Therrell B L, Jr., et al, Inborn errors of metabolism identified via newborn screening: Ten-year incidence data and costs of nutritional interventions for research agenda planning. Mol Genet Metab. 2014; 113(1-2):14-26] with mutations occurring throughout one of the three genes that make up the complex (BCKDHA, BCKDHB, DBT) resulting in MSUD [Nellis M M, Kasinski, et al.. Mol Genet Metab. 2003; 80(1-2):189-95; Ali E Z, Ngu L H. Mol Genet Metab Rep. 2018; 17:22-30; Henneke M, et al., Hum Mutat. 2003; 22(5):417; Rodriguez-Pombo P et al., Hum Mutat. 2006; 27(7):715] Certain populations have founder effects in one of these genes, for example the Mennonite population of Pennsylvania where a mutation in BCKDHA (c.1312T>A, p.Tyr438Asn) increases the frequency of MSUD to as high as 1 in 200. Morton D H, et al., Pediatrics. 2002; 109(6):999-1008; Strauss K A, et al, E G, et al. Branched-chain alpha-ketoacid dehydrogenase deficiency (maple syrup urine disease): Treatment, biomarkers, and outcomes. Mol Genet Metab. 2020.

BCKDH is responsible for the oxidative decarboxylation of the branched chain amino acids (BCAAs). Without BCKDH, the BCAAs leucine, isoleucine, and valine and their neurotoxic alpha-keto intermediates can build up in the blood and tissues. This disease gets its name from the distinctive sweet odor of affected patient's urine (branched-chain ketoaciduria). The majority of cases of MSUD present as the classic form in the immediate neonatal period. Classic MSUD patients have little to no enzyme activity (0-2% of normal), and the disease is characterized by neurological dysfunction and critical brain edema, which can result in death. A smaller subset of patients present with intermediate MSUD where they retain 3-30% of BCKDH activity resulting in less severe symptoms, including developmental delay, neurological impairment, failure to thrive, ketoacidosis, and seizures.

Inclusion of MSUD in newborn screening programs, particularly in areas with known founder effects has drastically decreased the time to treatment for patients with classic MSUD, has resulted in some improvement in outcomes. However, neurological damage following a metabolic crisis is still of major concern. There is currently no cure for MSUD, and treatment options are limited to strict dietary restriction of protein. Even with close monitoring of BCAA levels, MSUD patients are susceptible to a variety of neurological comorbidities, and may ultimately require a liver transplant. BCAA levels in MSUD patients that receive liver transplants are reduced within hours of transplant, but do remain approximately two-fold over normal levels. Strauss, cited above; Mazariegos G V, et al. J Pediatr. 2012; 160(1):116-21 e1. Following liver transplant, patients are no longer required to restrict protein intake. Mazariegos, cited above. Considering that liver provides only 9-13% of the total BCKDH activity in humans [Suryawan A, Hawes J W, Harris R A, Shimomura Y, Jenkins A E, Hutson S M. A molecular model of human branched-chain amino acid metabolism. Am J Clin Nutr. 1998; 68(1):72-81], it would suggest that correction of the same percentage of hepatocytes by gene therapy would be an effective treatment strategy.

Interestingly, those MSUD patients that choose to receive a liver transplant have recently been involved in domino liver transplantation surgeries, where their MSUD affected liver have been transplanted into patients without MSUD. [Mazariegos 2012, Suryawan 1998; Roda K M O, et al, Transplantation. 2019; 103(3):536-43; Badell I R, et al., Transplant Proc. 2013; 45(2):806-9] These patients do not develop MSUD as the majority (54-66%) of the oxidative BCKDH activity comes from skeletal muscle [Suryawan, cited above], and liver transplant recipients without MSUD are “protected” from BCKDH deficiency due to this muscle activity. Mazariegos, cited above. Therefore, skeletal muscle could provide an alternative site for gene therapy. There are several benefits from avoiding systemic administration of vector into the vasculature to target the liver, and instead injecting vector directly into muscle, including massively reduced involvement of pre-existing neutralizing antibodies to the AAV vector capsid. [Wang L, et al., Gene Ther. 2011; 22(11):1389-401; Greig J A, et al., Vaccine. 2016; 34(50):6323-9].

Currently, no cure exists for MSUD and treatment options are limited to carefully monitoring a restricted diet with the potential for liver transplantation, highlighting the unmet medical need to develop a novel therapeutic approach for this disease.

A need in the art exists for compositions and methods for efficient treatment of MSUD.

### SUMMARY OF THE INVENTION

In certain embodiments, a recombinant vector is provided which comprises one or more of an hDBT (MSUD-E2 coding) sequence of SEQ ID NO: 2 or a sequence at least 95% identical to SEQ ID NO: 2 which encodes SEQ ID NO: 1, a sequence having an BCKDHA (MSUD-E1A) coding sequence of SEQ ID NO:3 or a sequence at least 95% identical to SEQ ID NO: 3 which encodes SEQ ID NO: 4, or a sequence encoding an MSUD-E1B coding sequence of SEQ ID NO: 5 or a sequence at least 95% identical to SEQ ID NO: 5 which encodes SEQ ID NO: 6, an BCKDHB (MSUD-E1B coding) sequence. In certain embodiments, the recombinant vector comprises one or two of these MSUD-E1A, MSUD-E1B or MSUD-E2 coding sequences and optionally further comprises the coding sequences for the remaining protein from another source. In certain embodiments, the vector comprises sequences encoding all three of these proteins. In certain embodiments, the vector comprises sequences encoding two of these protein. In certain embodiments, the vector comprises sequences encoding one of these proteins.

In certain embodiments, a recombinant AAV (rAAV) comprising an AAV capsid and a vector genome packaged therein is provided, wherein the vector genome comprises an AAV 5′ inverted terminal repeat (ITR), an engineered hDBT nucleic acid sequence encoding a human MDUD-E2 subunit protein from a human branched-chain alpha-keto acid dehydrogenase (BCKDH), a regulatory sequence which directs expression of MSUD-E2 in a target cell, and an AAV 3′ ITR, wherein the hDBT (MSUD-E2 coding) sequence is at least 95% identical to SEQ ID NO: 2.

In certain embodiments, a recombinant AAV (rAAV) comprising an AAV capsid and a vector genome packaged therein is provided, wherein the vector genome comprises an AAV 5′ inverted terminal repeat (ITR), an engineered BCKDHA nucleic acid sequence encoding an MSUD-E1A subunit protein from a human branched-chain alpha-keto acid dehydrogenase (BCKDH) in a target cell, and an AAV 3′ ITR, wherein the BCKDHA (MSUD-E1A coding) sequence is at least 95% identical to SEQ ID NO: 3.

In certain embodiments, a recombinant AAV (rAAV) comprising an AAV capsid and a vector genome packaged therein is provided, wherein the vector genome comprises an AAV 5′ inverted terminal repeat (ITR), an engineered BCKDHB nucleic acid sequence encoding an MSUD-E1B subunit protein from a human branched-chain alpha-keto acid dehydrogenase (BCKDH) in a target cell, and an AAV 3′ ITR, wherein the BCKDHB (MSUD-E1B coding) sequence is at least 95% identical to SEQ ID NO: 5.

In certain embodiments, composition comprise one or more vectors comprising an hDBT (i.e., MSUD-E2) coding sequence of SEQ ID NO: 2 or a sequence at least 95% identical to SEQ ID NO: 2 which encodes SEQ ID NO: 1, a BCKDHA sequence (encoding an MSUD-E1A) of SEQ ID NO:3 or a sequence at least 95% identical to SEQ ID NO: 3 which encodes SEQ ID NO: 4, or a BCKDHB sequence (encoding an MSUD-E1B) of SEQ ID NO: 5 or a sequence at least 95% identical to SEQ ID NO: 5 which encodes SEQ ID NO: 6. In certain embodiments, a composition comprising and/or regimen for delivering all three of these hDBT (i.e., E2), BCKDHA (encoding MSUD-E1A) and BCKDHB (encoding MSUD-E1B) sequences. In other embodiments, a composition comprising and/or regimen for delivering coding sequences for all three of MSUD-E2, MSUD-E1A and MSUD-E1B, wherein only two of the sequences are selected from the engineered sequences above and the other coding sequence may be a wild-type coding sequence or a coding sequence from another source. In other embodiments, a composition comprising and/or regimen for delivering coding sequences for all three of MSUD-E2, MSUD-E1A and MSUD-E1B, wherein only one of the sequences are selected from the engineered sequences above and the other coding sequences may be wild-type sequence or from another source. Optionally, the composition may comprise a single vector carrying one, two or three of these coding sequences, separate vectors which differ from one another in the sequences they carry, or combinations. In certain embodiments, the vector is an rAAV expressing the MSUD-E1A subunit protein (e.g., rAAV. BCKDHA), MSUD-E1B subunit protein (e.g., rAAV. BCKDHB) or MSUD-E2 subunit protein (e.g., rAAV.hDBT), or combinations thereof is provided in a suspension buffer.

In certain embodiments, a composition may comprise an engineered MSUD-E2 mRNA sequence [SEQ ID NO: 30], an engineered MSUD-E1A mRNA sequence [SEQ ID NO: 31] and/or an engineered MSUD-E1B mRNA sequence [SEQ ID NO: 32]. In certain embodiments, one or more of the engineered mRNA sequences may be combined with one or more wild-type mRNA sequences, such that the composition, regimen and/or method of treatment comprises two or more, or all three of MSUD-E1A, MSUD-E1B and/or MSUD-E2. Optionally, a composition may comprise an mRNA corresponding to a wild-type MSUD-E1A, MSUD-E1B and/or MSUD-E2 protein in combination with one, two or all three engineered sequences provided herein.

In certain embodiments, a patient may be treated with an mRNA therapy as provided herein prior to viral vector—mediated gene therapy (e.g., rAAV.) with a composition comprising an rAAV or other viral vector as provided herein. In certain embodiments, a patient may be treated with mRNA therapy concurrently with rAAV-mediated therapy.

Use of a vector (e.g., rAAV) or an mRNA sequence as described herein in the manufacture of a medicament for treatment of Maple Syrup Urine Disease in a subject in need thereof is provided. In certain embodiments, the use provides for co-administration to the liver and muscle.

In certain embodiments, a method is provided for treating Maple Syrup Urine Disease comprising co-administering at least one gene therapy vector (e.g., comprising a human DBT gene encoding an MSUD-E2 subunit protein (e.g., rAAV.hDBT) under control of regulatory sequences which direct expression in liver and muscle. In certain embodiments, a method is provided for treating Maple Syrup Urine Disease comprising administering at least one gene therapy vector (e.g, comprising a sequence encoding an MSUD-E1A subunit protein (e.g., rAAV.DBCKHA) under control of regulatory sequences which direct expression in liver and muscle. In certain embodiments, a method for treating Maple Syrup Urine Disease comprising administering at least one gene therapy (e.g., an rAAV vector stock comprising a sequence encoding an MSUD-E1B subunit protein under control of regulatory sequences which direct expression in liver and muscle. In certain embodiments, a single vector stock (e.g., rAAV) is used which has a promoter directing expression in both liver and muscle. In other embodiments, two or more different vector stocks are utilized.

Other aspects and advantages of the invention will be readily apparent from the following detailed description of the invention.

### BRIEF DESCRIPTION OF THE DRAWINGS

FIGS. 1A to 1C show a comparison between a classic MSUD (cMSUD) and intermediate MSUD (iMSUD) mouse model for mutations in DBT/E2 of BCKDH protein. FIG. 1A shows a schematic overview of genotype of cMSUD mouse model. FIG. 1B shows a schematic overview of genotype corresponding to the tet-activating knock in of E2 resulting in iMSUD mouse model. FIG. 1C shows a comparison of percent survival in mice of cMSUD versus iMSUD models.

FIGS. 2A to 2B further provided data generated in an iMSUD mouse model. FIG. 2A shows percent survival of untreated iMSUD mice. FIG. 2B shows levels of branched chain amino acids (BCAAs) leucine, isoleucine and valine, plotted as normalized to alanine in untreated iMSUD mice.

FIGS. 3A to 3F shows anti-DBT (E2) immunohistochemistry (IHC). FIG. 3A shows a representative IHC anti-DBT staining in wild type mouse model with a human E2 knock in only. FIG. 3B a representative IHC anti-DBT staining in heterozygous mouse model with heterozygous for mouse E2 knockout (KO), and homozygous for human E2 knock in. FIG. 3C shows a representative IHC anti-DBT staining in hypomorph mouse model with mouse E2 KO, homozygous for human E2 knock in only. FIG. 3D shows a zoomed-in view of a representative IHC anti-DBT staining in wild type mouse model FIG. 3E shows a zoomed-in view of a representative IHC anti-DBT staining in heterozygous mouse model. FIG. 3F shows a representative IHC anti-DBT staining in hypomorph mouse model.

FIGS. 4A to 4D show a percent survival in various MSUD mouse models. FIG. 4A shows percent survival in E1α MSUD KO mouse model. FIG. 4B shows percent survival in E1β MSUD KO mouse model. FIG. 4C shows percent survival in E2 classic MSUD KO mouse model. FIG. 4A shows percent survival in E2 iMSUD mouse model.

FIGS. 5A to 5C shows efficacy of systemically administered gene therapy over time in the iMSUD mouse model. Treatment groups included: 3×10¹³ GC/kg of AAV8.TBG, 3×10¹¹ GC/kg of AAV9.CB7, 3×10² GC/kg of AAV9.CB7, and 3×10¹³ GC/kg of AAV9.CB7, all encoding human E2, and administered intravenously. FIG. 5A shows a plotted percent survival of iMSUD mice in treated and untreated groups. FIG. 5B shows plotted measurements of body weight followed during the in-life phase in treated and untreated groups. FIG. 5C shows plotted leucine levels as measured in ng per ml followed during in-life phase in treated and untreated groups. FIG. 5B and FIG. 5C values are presented as mean±SEM.

FIGS. 6A to 6C show a high dose vector with muscle-specific promoter increases body weight in the iMSUD mouse model, but has limited effect on survival and serum leucine levels. Treatment groups included: 3×10² GC/kg of AAV8.tMCK and 3×10¹³ GC/kg of AAV9.tMCK administered intramuscularly, and 3×10² GC/kg of AAV9.tMCK and 3×10¹³ GC/kg of AAV9.tMCK administered intravenously, and all encoding E2. FIG. 6A shows a plotted percent survival of iMSUD mice in treated and untreated groups. FIG. 6B shows plotted measurements of body weight followed during the in-life phase in treated and untreated groups. FIG. 6C shows plotted leucine levels as measured in ng per ml followed during in-life phase in treated and untreated groups. FIG. 6B and FIG. 6C values are presented as mean±SEM.

FIGS. 7A to 7C show a dose-dependent effect of intramuscularly administered vector on serum biomarker, but not on body weight in the intermediate mouse model of MSUD. Treatment groups included: 3×10¹¹ GC/kg of AAV9.CB7, 3×10¹² GC/kg of AAV9.CB7, and 3×10¹³ GC/kg of AAV9.CB7, all encoding human E2, and administered intramuscularly. FIG. 7A shows a plotted percent survival of iMSUD mice in treated and untreated groups. FIG. 7B shows plotted measurements of body weight followed during the in-life phase in treated and untreated groups. FIG. 7C shows plotted leucine levels as measured in ng per ml followed during in-life phase in treated and untreated groups. FIG. 7B and FIG. 7C values are presented as mean±SEM.

FIGS. 8A and 8B show enhanced RNA expression per vector genome copy from the CB7 promoter in the iMSUD mouse model. FIG. 8A shows DNA and RNA expression measurements from harvested liver tissues following necropsy. FIG. 8B shows DNA and RNA expression measurements from extracted muscle tissue following necropsy.

FIGS. 9A to 9D show an enhanced survival in gene therapy treated iMSUD mice in response to challenge with a high protein diet. Treatment groups included: 3×10¹¹ GC/kg of AAV9.CB7, 3×10² GC/kg of AAV9.CB7, and 3×10¹³ GC/kg of AAV9.CB7, all encoding human E2, and administered intramuscularly. FIG. 9A shows percent survival in iMSUD mice following challenge with the high protein diet. FIG. 9B shows plotted body weight of iMSUD mice from vector administration (day −14) through initiation of high protein diet challenge (day 0) to end of study (day 7). FIG. 9C shows a percentage change in body weight following challenge with the high protein diet. FIG. 9D shows serum leucine levels following high protein diet challenge. FIG. 9B and FIG. 9D values are presented as mean SEM.

FIGS. 10A to 10B show serum leucine levels in iMSUD mice following injection with rAAV9.CB7 at doses of 3×10¹² and 3×10¹³ GC/kg, as indicated, encoding a wild-type (WT) or an engineered DBT/E2 protein. FIG. 10A shows serum leucine levels following intramuscular injection with rAAV as specified. FIG. 10B shows serum leucine levels following intravenous injection with rAAV, as specified.

FIG. 11 shows an extended survival of classic MSUD mice following intravenous LNP encapsulated mRNA administration.

FIGS. 12A to 12D show an LNP encapsulated mRNA (E1a/E1b/E2; 2mpk) administered (weekly or biweekly) intravenously extends survival, increases body weight, and reduced serum leucine levels of cMSUD mice. FIG. 12A shows plotted body weight during in-life phase of the study up until day 14. FIG. 12B shows plotted body weight during in-life phase of the study up until day 21. FIG. 12C shows plotted body weight during in-life phase of the study up until day 42. FIG. 12D shows plotted serum leucine levels from harvested blood sample of a mouse euthanized at 24-hours post LNP injection.

FIG. 13 shows a comparison of percent survival of cMSUD (E2 KO) and E1a MSUD KO mice with respect to rescuing of acute crisis in newborns following triple LNP (E1a/E1b/E2 LNP) injections at 1, 2, and 3mpk.

FIGS. 14A to 14D show chronic therapy study with intravenously administered mRNA-LNP in adult iMSUD mice. Treatment groups included: E2 only (1 mpk), E2.GFP (0.5 and 1 mpk), E1a/E1b/E2 (0.2, 0.5, and 1 mpk), GFP (1 mpk). FIG. 14A shows a plotted percent survival of iMSUD mice in treated and untreated groups. FIG. 14B shows plotted measurements of body weight followed during the in-life phase in treated and untreated groups. FIG. 14C shows plotted leucine level values as normalized to alanine followed during in-life phase in treated and untreated groups. FIG. 14D shows plotted RNA levels of DBT/E2 expression as evaluated by RT-qPCR from extracted liver tissue harvested at necropsy.

FIGS. 15A to 15E show chronic therapy study with intravenously administered mRNA-LNP in newborn iMSUD mice. Treatment groups included: E1a/E1b/E2 (1 mpk) and GFP (1 mpk). Mice were administered newborn formulation of LNP at day 0 and 3, following an injection with adult formulation starting on day 7, and administered weekly or biweekly. FIG. 15A shows a plotted percent survival of iMSUD mice in treated and untreated groups. FIG. 15B shows plotted measurements of body weight followed during the in-life phase in treated and untreated groups. FIG. 15C shows plotted leucine level values (ng/mL) followed during in-life phase in treated and untreated groups. FIG. 15D shows plotted leucine level values (ng/ml) as measured at 24- and 48-hours post-injection. FIG. 15E shows plotted RNA levels of DBT/E2 and E1a expression as evaluated by RT-qPCR from extracted liver tissue harvested at necropsy.

### DETAILED DESCRIPTION OF THE INVENTION

Regimens and compositions useful for the treatment of Maple Syrup Urine Disease (MSUD) and/or alleviating symptoms of MSUD are provided herein. Compositions for delivery to both muscle and liver, has been found to have an improved therapeutic effect as compared to prior treatments which targeted only a single tissue type. This effect may be observed regardless of viral or non-viral gene therapy vector selected.

In certain embodiments, composition comprise one or more vectors comprising an E2 coding sequence (hDBT, or hDBTco) of SEQ ID NO: 2 or a sequence at least 95% identical to SEQ ID NO: 2 which encodes SEQ ID NO: 1, a DBCKHA sequence (encoding E1A) of SEQ ID NO:3 or a sequence at least 95% identical to SEQ ID NO: 3 which encodes SEQ ID NO: 4, or a DBCKHA sequence (encoding an E1B) of SEQ ID NO: 5 or a sequence at least 95% identical to SEQ ID NO: 5 which encodes SEQ ID NO: 6. In certain embodiments, a composition comprising and/or regimen for delivering all three of these MSUD-E2, -E1A and -E1B coding sequences. In other embodiments, a composition comprising and/or regimen for delivering coding sequences for all three of MSUD-E2, -E1A and -E1B, wherein only two of the sequences are selected from the engineered sequences above and the other coding sequence may be a wild-type coding sequence or a coding sequence from another source. In other embodiments, a composition comprising and/or regimen for delivering coding sequences for all three of MSUD-E2, MSUD-E1A and MSUD-E1B, wherein only one of the sequences are selected from the engineered sequences above and the other coding sequences may be wild-type sequence or from another source.

In certain embodiments, a pharmaceutical composition the vector is comprising an rAAV expressing the MSUD-E1A subunit protein, MSUD-E1B subunit protein or MSUD-E2 subunit protein, or combinations thereof is provided in a suspension buffer.

In certain embodiments, a composition may comprise an engineered MSUD-E2 mRNA sequence [SEQ ID NO: 30 or a sequence at least 95% identical thereto], an engineered MSUD-E1A mRNA sequence [SEQ ID NO: 31 or a sequence at least 95% identical thereto] and/or an engineered MSUD-E1B mRNA sequence [SEQ ID NO: 32 or a sequence at least 95% identical thereto]. In certain embodiments, one or more of the engineered mRNA sequences may be combined with one or more wild-type mRNA sequences, such that the composition, regimen and/or method of treatment comprises two or more, or all three of MSUD-E1A, E1B and/or E2.

Provided herein are therapies involving expression of an MSUD-E2 subunit protein of a branched-chain alpha-keto acid dehydrogenase (BCKDH) from a skeletal muscle-targeted rAAV.DBT (E2) vector, a liver-targeted rAAV.DBT (E2) vector, a vector that expresses in both skeletal muscle and liver. In other embodiments, the therapies involve additionally or alternatively delivering an mRNA encoding E2 encapsulated in an LNP formulation. Also provided herein are combination therapies wherein an MSUD-E1A subunit of BCKDH and/or an MSUD-E1B subunit of the BCKDH complex is expressed from muscle and/or liver following viral vector (e.g., AAV)-mediated delivery targeted to these tissues.

In addition, provided herein is a combination therapy wherein all three subunit proteins (MSUD-E1A, MSUD-E1B, and MSUD-E2) of the BCKDH complex expressed from muscle and/or liver following rAAV-mediated delivery and/or mRNA-mediated delivery targeted to these tissues. Further provided is a pharmaceutical composition comprising a rAAV as described herein in a formulation buffer, and a method of treating a human subject diagnosed with MSUD.

In certain embodiments, a vector (e.g, rAAV) comprises a DBT nucleic acid sequence encoding an MSUD-E2 subunit protein from branched-chain alpha-keto acid dehydrogenase (BCKDH). In certain embodiments, the MSUD-E2 subunit protein has the amino acid sequence of the transit peptide and mature chain of SEQ ID NO: 1 or a sequence at least 95% identical thereto. In certain embodiments, the transit peptide of the MSUD-E2 subunit is replaced with an exogenous transit peptide. For example, all or a portion of amino acids 1 to 61 of SEQ ID NO: 1 is replaced and a chimeric protein comprising an exogenous transit peptide located at the N-terminus of the mature chain (e.g., aa 62 to 482 of SEQ ID NO: 1). In certain embodiments, the native human coding sequence for the transit peptide and/or for the mature chain is used in the compositions and methods provided herein. In some embodiments, the engineered coding sequence of SEQ ID NO: 2 is selected or a sequence at least about 80%, 85%, 90%, 91%, 92%, 93%, 94%, 95%, 96%, 97%, 98%, 99%, or 99.9%) identical thereto which encodes SEQ ID NO: 1. In other embodiments, the engineered coding sequence of SEQ ID NO: 2 is selected or a sequence at least 95% identical thereto.

In certain embodiments, a composition and/or non-viral vector may comprise an engineered MSUD-E2 mRNA sequence of SEQ ID NO: 30 or a sequence at least 80%, 85%, 90%, 91%, 92%, 93%, 94%, 95%, 96%, 97%, 98%, 99%, or 99.9% identical thereto corresponding to the mature protein or the full-length mature MSUD-E2 mRNA. In certain embodiments, a composition and/or non-viral vector may comprise an engineered MSUD-E1A mRNA sequence of SEQ ID NO: 31 or a sequence at least 80%, 85%, 90%, 91%, 92%, 93%, 94%, 95%, 96%, 97%, 98%, 99%, or 99.9% identical thereto corresponding to the mature protein or the full-length mature MSUD-E1A mRNA. In certain embodiments, a composition and/or non-viral vector may comprise an engineered MSUD-E1B mRNA sequence of SEQ ID NO: 32 or a sequence at least 80%, 85%, 90%, 91%, 92%, 93%, 94%, 95%, 96%, 97%, 98%, 99%, or 99.9% identical thereto corresponding to the mature protein or the full-length mature MSUD-E1A mRNA. In certain embodiments, one or more of the engineered mRNA sequences may be combined with one or more wild-type mRNA sequences, such that the composition, regimen and/or method of treatment comprises two or more, or all three of MSUD-E1A, MSUD-E1B, and/or MSUD-E2.

In certain embodiments, a vector (e.g., rAAV) comprises a nucleic acid sequence encoding an MSUD-E1A subunit protein from BCKDH. In certain embodiments, the E1A subunit protein has the amino acid sequence of SEQ ID NO: 4. In certain embodiments, the native human BCKDHA coding sequence for the transit peptide and/or for the mature chain is used in the compositions and methods provided herein. In some embodiments, the engineered BCKDHA sequence of SEQ ID NO: 3 is selected or a sequence at least about 80%, 85%, 90%, 91%, 92%, 93%, 94%, 95%, 96%, 97%, 98%, 99%, or 99.9%) identical thereto and encodes SEQ ID NO: 4. In other embodiments, the engineered coding sequence of SEQ ID NO: 3 or a sequence at least 95% identical thereto is selected.

In certain embodiments, a vector (e.g., rAAV) comprises a nucleic acid sequence encoding an MSUD-E1B subunit protein from BCKDH. In certain embodiments, the MSUD-E1B subunit protein has the amino acid sequence of SEQ ID NO: 6. In certain embodiments, the native BCKDHB human coding sequence for the transit peptide and/or for the mature chain is used in the compositions and methods provided herein. In some embodiments, the engineered BCKDHB sequence of SEQ ID NO: 5 is selected or a sequence at least about 80%, 85%, 90%, 91%, 92%, 93%, 94%, 95%, 96%, 97%, 98%, 99%, or 99.9%) identical thereto and which encodes SEQ ID NO: 6. In other embodiments, the engineered coding sequence of SEQ ID NO: 5 or a sequence at least 95% identical thereto is selected.

In certain embodiments, a vector (e.g., rAAV) comprises the vector genome is nt 1 to nt 3428 of SEQ ID NO: 20, or nt 1 to nt 2981 of SEQ ID NO: 22, or nt 1 to nt 3538 of SEQ ID NO: 24, or nt 1 to nt 3811 of SEQ ID NO: 26, or nt 1 to nt 3027 of SEQ ID NO: 28 or a nucleic acid sequence at least about 80%, 85%, 90%, 91%, 92%, 93%, 94%, 95%, 96%, 97%, 98%, 99%, or 99.9%) identical thereto.

In certain embodiments, the compositions and methods described herein involve nucleic acid sequences, expression cassettes, vectors (e.g., recombinant viruses or LNPs), other compositions and methods for expression of a functional human MSUD-E2 (hE2). In another embodiment, the compositions and methods described herein involve nucleic acid sequences, expression cassettes, vectors, recombinant viruses, host cells, other compositions and methods for production of a composition comprising the DBT nucleic acid sequence encoding a functional hE2. In yet another embodiment, the compositions and methods described herein involve nucleic acid sequences, expression cassettes, vectors, recombinant viruses, other compositions and methods for delivery of the DBT nucleic acid sequence encoding a functional hE2 to a subject for the treatment of MSUD. In one embodiment, the compositions and methods described herein are useful for providing a therapeutic level of E2 into the muscle, liver, or muscle and liver. In certain embodiments, the methods involves delivery of the engineered hDBT sequence of SEQ ID NO: 2 (encoding MSUD-E2) or a sequence identical thereto as provided herein or an mRNA sequence of SEQ ID NO:30 or a sequence identical thereto and/or combinations thereof.

In certain embodiments, the compositions and methods described herein involve nucleic acid sequences, expression cassettes, vectors (e.g., recombinant viruses or LNPs), other compositions and methods for expression of a functional human E1A (hE1A). In another embodiment, the compositions and methods described herein involve nucleic acid sequences, expression cassettes, vectors, recombinant viruses, host cells, other compositions and methods for production of a composition comprising the BCKDHA nucleic acid sequence encoding a functional hE1A. In yet another embodiment, the compositions and methods described herein involve nucleic acid sequences, expression cassettes, vectors (e.g., recombinant viruses or LNPs), other compositions and methods for delivery of the BCKDHA nucleic acid sequence encoding a functional hE1A to a subject for the treatment of MSUD. In one embodiment, the compositions and methods described herein are useful for providing a therapeutic level of MSUD-E1A into the muscle, liver or muscle and liver. In certain embodiments, the methods involves delivery of the engineered BCKDHA sequence of SEQ ID NO: 3 (encoding MSUD-E1A) or a sequence identical thereto as provided herein or an mRNA sequence of SEQ ID NO:31 or a sequence identical thereto and/or combinations thereof.

In certain embodiments, the compositions and methods described herein involve nucleic acid sequences, expression cassettes, vectors, recombinant viruses, other compositions and methods for expression of a functional human MSUD-E1B (hE1B). In another embodiment, the compositions and methods described herein involve nucleic acid sequences, expression cassettes, vectors, recombinant viruses, host cells, other compositions and methods for production of a composition comprising the BCKDHB nucleic acid sequence encoding a functional hE1B. In yet another embodiment, the compositions and methods described herein involve BCKDHB nucleic acid sequences, expression cassettes, vectors, recombinant viruses, other compositions and methods for delivery of the BCKDHB nucleic acid sequence encoding a functional hE1B to a subject for the treatment of MSUD. In one embodiment, the compositions and methods described herein are useful for providing a therapeutic level of MSUD-E1B into the muscle, liver, or muscle and liver. In certain embodiments, the methods involves delivery of the engineered BCKDHB sequence of SEQ ID NO: 5 (encoding MSUD-E1B) or a sequence identical thereto as provided herein or an mRNA sequence of SEQ ID NO:32 or a sequence identical thereto and/or combinations thereof.

In certain embodiments, one or more vectors (e.g., one or more viral (e.g., rAAV) or non-viral (e.g., LNP)) described herein deliver hDBT, BCKDHA, and BCKDHB (and MSUD-E2, MDUD-E1A and/or MSUD-E1B) to the muscle and liver. In certain embodiments, one or more vectors will target the muscle for expression of the subunit protein(s). In certain embodiments, one or vectors will target the liver for expression of the subunit protein(s). These vectors may be formulated separately or admixed and delivered together. In certain embodiments, the vector are formulated separately and delivered sequentially. For example, a patient may receive non-viral gene therapy (e.g., via an LNP, naked DNA, peptide, or liposomal delivery systems) at a younger age and then a viral vector—mediated gene therapy upon reaching a threshold age. In some embodiments, the patient may receive non-viral gene therapy through the age of 1 year, up through age 3, through age 12, through age 18. In certain embodiments, viral-mediated gene therapy may be administered to an infant, to a patient after 3 years of age, after 12 years of age, after 18 years of age, or at another suitable age.

As used herein, the term “a therapeutic level” means an enzyme activity at least about 5%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of a healthy control. It will be understood that when reference is made therein to delivery of a E1A, E1B and/or E2, that expression of a therapeutic level of the protein is considered delivery of a functional subunit protein. Suitable assays for measuring the enzymatic activity of an MSUD subunit protein are described herein. In some embodiments, such therapeutic levels of the one or more subunit protein may result in alleviation of the MSUD related symptom(s); reversal of certain MSUD-related symptoms and/or prevention of progression of MSUD-related certain symptoms; or any combination thereof.

As used herein, “a healthy control” refers to a subject or a biological sample therefrom, wherein the subject does not have an MSUD. The healthy control can be from one subject. In another embodiment, the healthy control is a pool of multiple subjects.

As used herein, the term “biological sample” refers to any cell, biological fluid or tissue. Suitable samples for use in this invention may include, without limitation, whole blood, leukocytes, fibroblasts, serum, urine, plasma, saliva, bone marrow, cerebrospinal fluid, amniotic fluid, and skin cells. Such samples may further be diluted with saline, buffer or a physiologically acceptable diluent. Alternatively, such samples are concentrated by conventional means.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

As used herein, “disease”, “disorder” and “condition” are Maple Syrup Urine Disease (MSUD). MSUD has also been called BCKD Deficiency, branched-chain ketoacid dehydrogenase deficiency, and/or branched-chain ketoaciduria.

As used herein, the term “MSUD-related symptom(s)” or “symptom(s)” refers to symptom(s) found in MSUD patients as well as in MSUD animal models. Such symptoms include, e.g., lethargy, poor appetite, weight loss, weak sucking ability, irritability, a distinctive maple sugar odor in earwax, sweat, and urine, irregular sleep patterns, alternating episodes of hypertonia (muscle rigidity), and hypotonia (muscle limpness).

“Patient” or “subject” as used herein means a male or female human, dogs, and animal models used for clinical research. In one embodiment, the subject of these methods and compositions is a human diagnosed with MSUD. In certain embodiments, the human subject of these methods and compositions is a prenatal, a newborn, an infant, a toddler, a preschool, a grade-schooler, a teen, a young adult or an adult. In a further embodiment, the subject of these methods and compositions is a pediatric MSUD patient.

“Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention. It should be understood that while various embodiments in the specification are presented using “comprising” language, under various circumstances, a related embodiment is also described using “consisting of” or “consisting essentially of” language.

It is to be noted that the term “a” or “an”, refers to one or more, for example, “a vector”, is understood to represent one or more rAAV(s) or another specified vector. As such, the terms “a” (or “an”), “one or more,” and “at least one” is used interchangeably herein.

As used herein, the term “about” means a variability of plus or minus 10% from the reference given, unless otherwise specified.

### 1. BRANCHED-CHAIN ALPHA-KETO ACID DEHYDROGENASE (BCKDH) SUBUNIT PROTEINS

The BCKDH complex is composed of multiple subunit proteins. Deficiency of the E1α, E1β or E2 subunits result in MSUD. The E1a (also termed herein E1A or E1a), E1β (also termed herein E1B or E1b) and E2 (also termed herein DBT/E2) subunits are encoded by the BCKDHA, BCKDHB and DBT genes, respectively. It will be understood that when reference is made therein to delivery of a BCKDHA gene (encoding MSUD-E1A), BCKDHB gene (encoding MSUD-E1B) and/or hDBT gene (encoding MSUD-E2), that reference is to that of a coding sequence which expresses a functional subunit protein, regardless of whether the term “functional” is specified in the specific passage in the specification.

As used herein, the term “functional E2” means an enzyme having the amino acid sequence of the full-length wild-type (native) human E2 (as shown in SEQ ID NO: 1 and UniProtKBSwiss-Prot P11182.3), a variant thereof, a mutant thereof with a conservative amino acid replacement, a fragment thereof, a full-length or a fragment of any combination of the variant and the mutant with a conservative amino acid replacement, which provides at about 5%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of a normal human E2. In one embodiment, a functional MSUD-hE2 refers to an E2 protein with sequence of at least the mature chain in SEQ ID NO: 1.

As used herein, the term “functional E1A” means an enzyme having the amino acid sequence of the full-length wild-type (native) human E1A (as shown in SEQ ID NO: 4), a variant thereof, a mutant thereof with a conservative amino acid replacement, a fragment thereof, a full-length or a fragment of any combination of the variant and the mutant with a conservative amino acid replacement, which provides a about 5%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of normal human E1A. In one embodiment, a functional E1B refers to a wild-type MSUD-hE1A protein with sequence of SEQ ID NO: 4.

As used herein, the term “functional E1B” means an enzyme having the amino acid sequence of the full-length wild-type (native) human E1B (as shown in SEQ ID NO: 6), a variant thereof, a mutant thereof with a conservative amino acid replacement, a fragment thereof, a full-length or a fragment of any combination of the variant and the mutant with a conservative amino acid replacement, which provide about 5%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of normal human E1B. In one embodiment, a functional E1B refers to a wild-type MUSD-hE1B protein with sequence of SEQ ID NO: 6.

In this specification, these subunits protein may be referred to as MSUD-E1A, MSUD-E1B, or MSUD-E2 to distinguish them from subunits from other sources.

As used herein, the “conservative amino acid replacement” or “conservative amino acid substitutions” refers to a change, replacement or substitution of an amino acid to a different amino acid with similar biochemical properties (e.g. charge, hydrophobicity and size), which is known by practitioners of the art. Also see, e.g. French et al. What is a conservative substitution? Journal of Molecular Evolution, March 1983, Volume 19, Issue 2, pp 171-175 and YAMPOLSKY et al. The Exchangeability of Amino Acids in Proteins, Genetics. 2005 August; 170(4): 1459-1472, each of which is incorporated herein by reference in its entirety.

A variety of assays exist for measuring MSUD-E1A, -E1B and/or-E2 expression and activity levels by conventional methods.

A nucleic acid refers to a polymeric form of nucleotides and includes RNA, mRNA, cDNA, genomic DNA, peptide nucleic acid (PNA) and synthetic forms and mixed polymers of the above. A nucleotide refers to a ribonucleotide, deoxynucleotide or a modified form of either type of nucleotide (e.g., a peptide nucleic acid oligomer). The term also includes single- and double-stranded forms of DNA. The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules.

In certain embodiments, the nucleic acid molecules encoding a functional human MSUD-E1A, E1B and/or E2 and other constructs encompassed in the compositions and methods described herein and useful in generating expression cassettes and vector genomes engineered for expression in yeast cells, insect cells or mammalian cells, such as human cells. In certain embodiments, a nucleic acid sequence comprises an expression cassette is nt 1 to nt 3428 of SEQ ID NO: 20, or nt 1 to nt 2981 of SEQ ID NO: 22, or nt 1 to nt 3538 of SEQ ID NO: 24, or nt 1 to nt 3811 of SEQ ID NO: 26, or nt 1 to nt 3027 of SEQ ID NO: 28 or a nucleic acid sequence at least about 70% (e.g., at least about 75%, 80%, 85%, 90%, 91%, 92%, 93%, 94%, 95%, 96%, 97%, 98%, 99%, or 99.9%) identical thereto.

Methods are known and have been described previously (e.g. WO 96/09378). A sequence is considered engineered if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in www.kazusa.jp/codon. Preferably more than one non-preferred codon, preferably most or all non-preferred codons, are replaced by codons that are more preferred. Preferably the most frequently used codons in an organism are used in an engineered sequence. Replacement by preferred codons generally leads to higher expression. It will also be understood by a skilled person that numerous different nucleic acid molecules can encode the same polypeptide as a result of the degeneracy of the genetic code. It is also understood that skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g. GeneArt, GenScript, Life Technologies, Eurofins). In certain embodiments, the nucleic acid sequences are mRNA transcripts and can generated using routine in vitro transcription reactions in laboratory (i.e. MegaScript T7 Transcription kit by Thermo Fisher) or by service companies having business in the field (e. g. TriLink).

In one aspect, the E2 coding sequence (DBT) is an engineered nucleic acid sequence (DBTco). In one embodiment, the engineered sequence is useful to improve production, transcription, expression or safety in a subject. In another embodiment, the engineered sequence is useful to increase efficacy of the resulting therapeutic compositions or treatment. In a further embodiment, the engineered sequence is useful to increase the efficacy of the functional MSUD-E1A, -E1B and/or -E2 protein being expressed, but may also permit a lower dose of a therapeutic reagent that delivers the functional protein to increase safety.

By “engineered” is meant that the nucleic acid sequences encoding a functional E2, E1A or E1B protein described herein are assembled and placed into any suitable genetic element, e.g., naked DNA, phage, transposon, cosmid, episome, etc., which transfers the E2 (E1A or E1B) sequences carried thereon to a host cell, e.g., for generating non-viral delivery systems (e.g., RNA-based systems, naked DNA, or the like), or for generating viral vectors in a packaging host cell, and/or for delivery to a host cells in a subject. In one embodiment, the genetic element is a vector. In one embodiment, the genetic element is a plasmid. In one embodiment, the genetic element is an mRNA transcript. The methods used to make such engineered constructs are known to those with skill in nucleic acid manipulation and include genetic engineering, recombinant engineering, in vitro and synthetic techniques. See, e.g., Green and Sambrook, Molecular Cloning: A Laboratory Manual, Cold Spring Harbor Press, Cold Spring Harbor, N.Y. (2012).

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Multiple sequence alignment programs are also available for nucleic acid sequences. Examples of such programs include, “Clustal Omega”, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available (e.g., BLAST, ExPASy; Clustal Omega; FASTA; using, e.g., Needleman-Wunsch algorithm, Smith-Waterman algorithm). Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used herein, the phrases “ameliorate a symptom”, “improve a symptom” or any grammatical variants thereof, refer to reversal of an MSUD-related symptoms, showdown or prevention of progression of an MSUD-related symptoms. In one embodiment, the amelioration or improvement refers to the total number of symptoms in a patient after administration of the described composition(s) or use of the described method, which is reduced by about 5%, about 10%, about 20%, about 30%, about 40%, about 50%, about 60%, about 70%, about 80%, about 90%, about 95% compared to that before the administration or use. In another embodiment, the amelioration or improvement refers to the severity or progression of a symptom after administration of the described composition(s) or use of the described method, which is reduced by about 5%, about 10%, about 20%, about 30%, about 40%, about 50%, about 60%, about 70%, about 80%, about 90%, about 95% compared to that before the administration or use.

It should be understood that the compositions comprising an BCKDHA gene, an BCKDHB gene and/or a DBT gene encoding a functional E2 protein and E2 coding sequences described herein are intended to be applied to E1A and/or E1B, other compositions, regimens, aspects, embodiments and methods described across the Specification.

### 2. EXPRESSION CASSETTE

In one aspect, provided is an expression cassette comprising an engineered DBT nucleic acid sequence encoding a functional E2, and a regulatory sequence which directs expression thereof. In one embodiment, provided is an expression cassette comprising an engineered nucleic acid sequence as described herein which encodes a functional hE2, and a regulatory sequence which directs expression thereof. In one embodiment, the hDBT (hE2 coding sequence) is at least 95% identical to SEQ ID NO: 2. In a further embodiment, the hDBT (hE2 coding sequence) is SEQ ID NO: 2. In one embodiment, provided is an expression cassette comprising an engineered BCKDHA nucleic acid sequence as described herein which encodes a E1A, and a regulatory sequence which direct expression thereof. In one embodiment, the BCKDHA (E1A coding sequence) is at least 95% identical to SEQ ID NO: 3. In a further embodiment, the BCKDHA (E1A coding sequence) is SEQ ID NO: 3. In one embodiment, provided is an expression cassette comprising an engineered BCKDHB nucleic acid sequence as described herein which encodes a E1B, and a regulatory sequence which direct expression thereof. In one embodiment, the BCKDHB (E1B coding sequence) is at least 95% identical to SEQ ID NO: 5. In a further embodiment, the BCKDHB (E1B coding sequence) is SEQ ID NO: 5.

In one embodiment, the regulatory sequence comprises a promoter. In a further embodiment, the regulatory sequence comprises a CB7 promoter. In one embodiment, the regulatory sequence further comprises a chicken beta-actin intron. In one embodiment, the regulatory sequence further comprises a rabbit globin poly A. In one embodiment, the regulatory sequence comprises a multicistronic element, wherein the element is an internal ribosome entry site (IRES), a furin-2a or Thosea asigna virus cleavage site (T2a), thereby allowing expression of two or more encoding constructs. In certain vectors, e.g., rAAV, fewer than three coding sequences are packaged into a single vector due to preference or packaging capacity and multiple different viral stock can be required for a desired composition or a therapeutic regimen. However, in other vectors, including, e.g., lentiviruses or non-viral vectors, a single nucleic acid may contain multiple MSUD-subunit coding sequences. In such embodiments, use of an IRES, F2A or T2A protein may be desired in a vector which comprises more than one of the MSUD-E2, E1A and/or E1B coding sequences, to permit expression from a single expression cassette.

As used herein, the term “expression” or “gene expression” refers to the process by which information from a gene is used in the synthesis of a functional gene product. The gene product may be a protein, a peptide, or a nucleic acid polymer (such as a RNA, a DNA or a PNA).

As used herein, an “expression cassette” refers to a nucleic acid polymer which comprises the hDBT (and/or BCKDHA and/or BCKDHB) coding sequences for a functional hE2 (and/or E1A and/or E1B), promoter, and may include other regulatory sequences therefor, which cassette may be packaged into a vector (e.g., rAAV). Optionally, an expression cassette (and a vector genome) may comprise one or more dorsal root ganglion (drg)-miRNA targeting sequences in the UTR, e.g., to reduce drg-toxicity and/or axonopathy. See, e.g., PCT/US2019/67872, filed Dec. 20, 2019, U.S. Provisional Patent Application No. 63/023,593, filed May 12, 2020, which is incorporated herein in its entirety.

As used herein, the term “regulatory sequence”, or “expression control sequence” refers to nucleic acid sequences, such as initiator sequences, enhancer sequences, and promoter sequences, which induce, repress, or otherwise control the transcription of protein encoding nucleic acid sequences to which they are operably linked.

As used herein, the term “operably linked” refers to both expression control sequences that are contiguous with the hDBT (and/or BCKDHA and/or BCKDHB) nucleic acid sequence encoding the functional E2, E1A, E1B proteins and/or expression control sequences that act in trans or at a distance to control the transcription and expression thereof.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g. a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector (e.g., rAAV), indicates that the protein or the nucleic acid is present with another sequence or subsequence which with which the protein or nucleic acid in question is not found in the same relationship to each other in nature.

In one embodiment, the expression cassette is designed for expression and secretion in a human subject. In one embodiment, the expression cassette is designed for expression in muscle. In one embodiment, the expression cassette is designed for expression in liver.

In certain embodiments, the expression cassette is designed for expression in both liver and muscle. In such embodiments, a constitutive promoter may be selected. In one embodiment, the promoter is a chicken 0-actin promoter. A variety of chicken beta-actin promoters have been described alone, or in combination with various enhancer elements (e.g., CB7 is a chicken beta-actin promoter with cytomegalovirus enhancer elements; a CAG promoter, which includes the promoter, the first exon and first intron of chicken beta actin, and the splice acceptor of the rabbit beta-globin gene; a CBh promoter, SJ Gray et al, Hu Gene Ther, 2011 September; 22(9): 1143-1153). Alternatively, other constitutive promoters may be selected.

Suitable promoters may be selected, including but not limited to a constitutive promoter, a tissue-specific promoter or an inducible/regulatory promoter. Example of a constitutive promoter is chicken beta-actin promoter. Examples of liver-specific promoters may include, e.g., thyroid hormone-binding globulin (TBG), albumin, Miyatake et al., (1997) J. Virol., 71:5124-32; hepatitis B virus core promoter, Sandig et al., (1996) Gene Ther., 3:1002-9; or human alpha 1-antitrypsin, phosphoenolpyruvate carboxykinase (PECK), or alpha-fetoprotein (AFP), Arbuthnot et al., (1996) Hum. Gene Ther., 7:1503-14). Preferably, such promoters are of human origin. Examples of muscle-specific promoters may include, e.g., the muscle creatine kinase (MCK) promoter and truncated forms thereof. See, e.g., B. Wang, et al, Gene Therapy volume 15, pages 1489-1499 (2008). See, also, muscle-specific transcriptional cis-regulatory modules (CRMs), such as those described S. Sarcare, et al, (January 2019) Nat Commun. 2019; 10: 492. Alternatively, a regulatable promoter may be selected. In one embodiment, a regulatable promoter is a regulatable system wherein may be selected from a tet-on/off system, a tetR-KRAB system, a mifepristone (RU486) regulatable system, a tamoxifen-dependent regulatable system, a rapamycin—regulatable system, or an ecdysone-based regulatable system. See, e.g., WO 2011/126808B2, incorporated by reference herein.

In one embodiment, the regulatory sequence further comprises an enhancer. In one embodiment, the regulatory sequence comprises one enhancer. In another embodiment, the regulatory sequence contains two or more expression enhancers. These enhancers may be the same or may be different. For example, an enhancer may include an Alpha mic/bik enhancer or a CMV enhancer. This enhancer may be present in two copies which are located adjacent to one another. Alternatively, the dual copies of the enhancer may be separated by one or more sequences.

In one embodiment, the regulatory sequence further comprises an intron. In a further embodiment, the intron is a chicken beta-actin intron. Other suitable introns include those known in the art may by a human β-globulin intron, and/or a commercially available Promega® intron, and those described in WO 2011/126808.

In one embodiment, the regulatory sequence further comprises a Polyadenylation signal (polyA). In a further embodiment, the polyA is a rabbit globin poly A. See, e.g., WO 2014/151341. Alternatively, another polyA, e.g., a human growth hormone (hGH) polyadenylation sequence, a bovine growth hormone polyadenylation sequence, an SV40 polyA, or a synthetic polyA may be included in an expression cassette.

In one embodiment the regulatory sequence further comprises a Woodchuck Hepatitis Virus (WHP) Posttranscriptional Regulatory Element (WRPE).

It should be understood that the compositions in the expression cassette described herein are intended to be applied to other compositions, regiments, aspects, embodiments and methods described across the Specification.

### 3. VECTOR

In one aspect, provided herein is a vector comprising an engineered hDBT nucleic acid sequence encoding a functional human E2 and a regulatory sequence which direct expression thereof in a target cell. In one embodiment, the hDBT (hE2 coding sequence) is at least 95% identical to SEQ ID NO: 2. In a further embodiment, the hDBT (hE2 coding sequence) is SEQ ID NO: 2. In one aspect, provided herein is a vector comprising an engineered BCKDHA nucleic acid sequence encoding a functional human E1A and a regulatory sequence which direct expression thereof in a target cell. In one embodiment, the BCKDHA (hE1A coding sequence) is at least 95% identical to SEQ ID NO: 3. In a further embodiment, the BCKDHA(hE1A coding sequence) is SEQ ID NO: 3. In one aspect, provided herein is a vector comprising an engineered BCKDHB nucleic acid sequence encoding a functional human E1B and a regulatory sequence which direct expression thereof in a target cell. In one embodiment, the BCKDHB (hE1B) coding sequence is at least 95% identical to SEQ ID NO: 5. In a further embodiment, the BCKDHB (hE1B) coding sequence is SEQ ID NO: 5. In certain embodiments, combinations of these vectors are used. In particularly desirable embodiments, replication-defective recombinant adeno-associated viruses carrying the MSUD subunit protein are used in the compositions and methods provided herein. In certain embodiments, a vector comprises a nucleic acid molecule of nucleic acid sequence of SEQ ID NO: 20, or SEQ ID NO: 22, or SEQ ID NO: 24, or SEQ ID NO: 26, or SEQ ID NO: 28.

A “vector” as used herein is a biological or chemical moiety comprising a nucleic acid sequence which can be introduced into an appropriate target cell for replication or expression of said nucleic acid sequence. Examples of a vector includes but not limited to a recombinant virus, a plasmid, Lipoplexes, a Polymersome, Polyplexes, a dendrimer, a cell penetrating peptide (CPP) conjugate, a magnetic particle, or a nanoparticle. In one embodiment, a vector is a nucleic acid molecule into which an exogenous or heterologous or engineered hDBT (and/or BCKDHA and/or BCKDHB) nucleic acid encoding a functional MSUD-E2 (and/or MSUD-E1A and/or MSUD-E1B, respectively) may be inserted, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

In one embodiment, the vector is a non-viral plasmid that comprises an expression cassette described thereof, e.g., “naked DNA”, “naked plasmid DNA”, RNA, mRNA, shRNA, RNAi, etc. Optionally the plasmid or other nucleic acid sequence is delivered via a suitable device, e.g., via electrospray, electroporation. In other embodiments, the nucleic acid molecule is coupled with various compositions and nano particles, including, e.g., micelles, liposomes, cationic lipid—nucleic acid compositions, poly-glycan compositions and other polymers, lipid and/or cholesterol-based—nucleic acid conjugates, and other constructs such as are described herein. See, e.g., WO2014/089486, US 2018/0353616A1, US2013/0037977A1, WO2015/074085A1, U.S. Pat. No. 9,670,152B2, and U.S. Pat. No. 8,853,377B2, X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiment, a non-viral vector is used for delivery of an mRNA transcript comprising an engineered hDBT nucleic acid sequence encoding a functional human MSUJ-E2 and a regulatory sequence which direct expression thereof in a target cell. In one embodiment, the hDBT (hE2 coding) sequence is at least 95% identical to SEQ ID NO: 30. In a further embodiment, the hDBT (hE2 coding) sequence is SEQ ID NO: 30. In one embodiment, a non-viral vector is used for delivery of an mRNA transcript comprising an engineered BCKDHA nucleic acid sequence encoding a functional human E1A and a regulatory sequence which direct expression thereof in a target cell. In one embodiment, the BCKDHA (hE1A coding) sequence is at least 95% identical to SEQ ID NO: 31. In a further embodiment, the BCKDHA (hE1A coding) sequence is SEQ ID NO: 31. In one embodiment, a non-viral vector is used for delivery of an mRNA transcript comprising an engineered BCKDHB nucleic acid sequence encoding a functional human E1B and a regulatory sequence which direct expression thereof in a target cell. In one embodiment, the BCKDHB (hE1B coding) sequence is at least 95% identical to SEQ ID NO: 32. In a further embodiment, the BCKDHB (hE1B coding) sequence is SEQ ID NO: 32. In certain embodiments, combinations of these vectors are used.

In certain embodiments, the regulatory sequence of an mRNA transcript comprises of a cap structure at 5′ end, an untranslated region at 5′ end (5′UTR), an untranslated region at 3′ end (3′UTR), and poly(A) tail at 3′end. In certain embodiments, the nucleic acid sequence of an mRNA transcript comprises modified nucleosides of 5-Methylcytosine, and/or pseudouridine. In certain embodiments, the 5′ cap is a modified 5′ cap analog. In certain embodiments, the poly(A) tail comprises of at least about 100 to at least about 250 adenylates. In one embodiment, the poly(A) tail is at least about 150 to at least about 200 adenylates. In certain embodiments, the mRNA transcript sequence is 5′cap-5′UTR-hE2-3′UTR-3′poly(A)tail. In certain embodiments, the mRNA transcript sequence is 5′cap-5′UTR-hE1A-3′UTR-3′poly(A)tail. In certain embodiments, the mRNA transcript sequence is 5′cap-5′UTR-hE1B-3′UTR-3′poly(A)tail.

In one example, a non-viral vector genome comprising an mRNA transcript contains, at a minimum, from 5′ to 3′, 5′ cap, an 5′ UTR, a nucleic acid sequence encoding at least one functional hMSUD-E2 (and/or hMSUD-E1A, and/or hMSUD-E1B), a 3′UTR and a poly(A) tail.

In some embodiments, the mRNA is delivered at an amount greater than about 0.5 mg/kg (e.g., greater than about 10 mg/kg, 1.5 mg/kg, 2.0 mg/kg, 2.5 mg/kg, 3.0 mg/kg, 4.0 mg/kg, 5.0 mg/kg, 6.0 mg/kg, 7.0 mg/kg 8.0 mg/kg, 9.0 mg/kg or 10.0 mg/kg) body weight of mRNA per dose. In some embodiments, the mRNA is delivered at an amount ranging from about 0.1-100 mg/kg (e.g. about 0.1-90 mg/kg, 0.1-80 mg/kg, 0.1-70 mg/kg, 0.1-60 mg/kg, 0.1-50 mg/kg, 0.1-40 mg/kg, 0.1-30 mg/kg, 0.1-20 mg/kg 0.1-10 mg/kg) body weight of mRNA per dose. In some embodiments, the mRNA is delivered at an amount of or greater than about 1 mg, 5 mg, 10 mg, 15 mg, 20 mg, 25 mg, 30 mg, 35 mg, 40 mg, 45 mg, 50 mg, 55 mg, 60 ng, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg per dose.

In certain embodiments, mRNA transcripts are encapsulated in a lipid nanoparticle (LNP). As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more mRNA to one or more target cells (e.g., liver and/or muscle). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a mRNA to a target cell. Useful lipid nanoparticles for mRNA comprise a cationic lipid to encapsulate and/or enhance the delivery of mRNA into the target cell that will act as a depot for protein production. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid, PEG-lipid and polymer forming a lipid bilayer around encapsulated mRNA (Kowalski et al., 2019, Mol. Ther. 27(4):710-728). In some embodiments, LNP comprises a cationic lipids (i.e. N-[1-(2,3-dioleoyloxy)propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino)esters (PBAEs). See, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference.

In certain embodiments, the vector described herein is a “replication-defective virus” or a “viral vector” which refers to a synthetic or artificial viral particle in which an expression cassette containing a nucleic acid sequence encoding a functional hE2, E1A or E1B is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the nucleic acid sequence encoding E2 flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant viral vector may be any suitable replication-defective viral vector, including, e.g., a recombinant adeno-associated virus (AAV), an adenovirus, a bocavirus, a hybrid AAV/bocavirus, a herpes simplex virus or a lentivirus.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

As used herein, the term “target cell” refers to any target cell in which expression of the functional E2, E1A and/or E1B is desired. In certain embodiments, the term “target cell” is intended to reference the cells of the subject being treated for MSUD. Examples of target cells may include, but are not limited to, a liver cell, skeletal muscle cell, and/or a stem cell. In certain embodiments, the vector is delivered to a target cell ex vivo. In certain embodiments, the vector is delivered to the target cell in vivo.

It should be understood that the compositions in the vector described herein are intended to be applied to other compositions, regiments, aspects, embodiments and methods described across the Specification.

### 4. ADENO-ASSOCIATED VIRUS (AAV)

In one aspect, provided herein is a recombinant AAV (rAAV) comprising an AAV capsid and a vector genome packaged therein. The rAAV is for use in the treatment of MSUD. In certain embodiments, the vector genome comprises an AAV 5′ inverted terminal repeat (ITR), an engineered hDBT nucleic acid sequence encoding a functional hE2 as described herein, a regulatory sequence which direct expression of hE2 in a target cell, and an AAV 3′ ITR. In one embodiment, the hDBT (hE2 coding) sequence is at least 95% identical to SEQ ID NO: 2. In a further embodiment, the hDBT (hE2) coding sequence is SEQ ID NO: 2. In certain embodiments, the regulatory sequence comprises a tissue-specific promoter (e.g., muscle or liver). In certain embodiments, a composition or regimen is designed to include a combination of AAV.hDBT (i.e., AAV.hE2) stocks, each having a different tissue-specific promoter. In certain embodiments, a vector genome is SEQ ID NO: 20 or is at least 95% identical to SEQ ID NO: 20. In certain embodiments, a vector genome is SEQ ID NO: 22 or is at least 95% identical to SEQ ID NO: 22. In certain embodiments, a vector genome is SEQ ID NO: 24 or is at least 95% identical to SEQ ID NO: 24. In certain embodiments, a vector genome is SEQ ID NO: 26 or is at least 95% identical to SEQ ID NO: 26. In certain embodiments, a vector genome is SEQ ID NO: 28 or is at least 95% identical to SEQ ID NO: 28.

The vector genome comprises an AAV 5′ inverted terminal repeat (ITR), an engineered BCKDHA nucleic acid sequence encoding a functional hE1A as described herein, a regulatory sequence which direct expression of hE1A in a target cell, and an AAV 3′ ITR. In one embodiment, the BCKDHA (hE1A coding) sequence is at least 95% identical to SEQ ID NO: 3. In a further embodiment, the BCKDHA (hE1A coding) sequence is SEQ ID NO: 3. In certain embodiments, the regulatory sequence comprises a tissue-specific promoter (e.g., muscle or liver). In certain embodiments, a composition or regimen is designed to include a combination of AAV.hBCKDHA stocks, each having a different tissue-specific promoter.

The vector genome comprises an AAV 5′ inverted terminal repeat (ITR), an engineered BCKDHB nucleic acid sequence encoding a functional hE1B as described herein, a regulatory sequence which direct expression of hE1B in a target cell, and an AAV 3′ ITR. In one embodiment, the BCKDHB (hE1B coding) sequence is at least 95% identical to SEQ ID NO: 5. In a further embodiment, the BCKDHB (hE1B coding) sequence is SEQ ID NO: 5. In certain embodiments, the regulatory sequence comprises a tissue-specific promoter (e.g., muscle or liver). In certain embodiments, a composition or regimen is designed to include a combination of AAV. BCKDHB stocks, each having a different tissue-specific promoter.

In a further embodiment, the regulatory sequence further comprises an enhancer. In one embodiment, the regulatory sequence further comprises an intron. In one embodiment, the regulatory sequence further comprises a poly A. In one embodiment, the AAV capsid is an AAV1 capsid. In certain embodiments, the AAV capsid is an AAV8 capsid. In certain embodiments, the AAV capsid is an AAV9 capsid. In one embodiment, the rAAV described herein is for use in the treatment of MSUD.

In one embodiment, the regulatory sequence is as described above. In one embodiment, the vector genome comprises an AAV 5′ inverted terminal repeat (ITR), an expression cassette as described herein, and an AAV 3′ ITR.

In one embodiment, provided is a rAAV comprising an AAV serotype 9 (AAV9) capsid and a vector genome comprises CB7.CI.hMSUD-E2.SV40 (SEQ ID NO: 26); TBG.hMSUD-E2.WRPE.BGH (SEQ ID NO: 24), TBG.PI.hMSUD-E1A.WRPE.BGH (SEQ ID NO: 20), TGB.PI.hMSUD-E2.BGH (SEQ ID NO: 22), or tMCK.PI.hMSUD-E2.SV40 (SEQ ID NO: 28). In one embodiment, provided is a rAAV comprising an AAV serotype 8 (AAV8) capsid and a vector genome comprises CB7.CI.hMSUD-E2.SV40; TBG.hMSUD-E2.WPRE.BGH, TBG.PI.hMSUD-E1A.WPRE.BGH, TGB.PI.hMSUD-E2.BGH, or tMCK.PI.hMSUD-E2.SV40. In one embodiment, provided is a rAAV comprising an AAV serotype 1 (AAV1) capsid and a vector genome comprises CB7.CI.hMSUD-E2.SV40; TBG.hMSUD-E2.WPRE.BGH, TBG.PI.hMSUD-E1A.WPRE.BGH, TGB.PI.hMSUD-E2.BGH, or tMCK.PI.hMSUD-E2.SV40. In certain embodiments, these vector genomes may be engineered into another AAV capsid and/or another vector. Additionally, or alternatively, other vector elements may be selected.

As used herein, a “vector genome” refers to the nucleic acid sequence packaged inside a vector. In one embodiment, the vector genome refers to the nucleic acid sequence packaged inside a rAAV capsid forming an rAAV vector. Such a nucleic acid sequence contains AAV inverted terminal repeat sequences (ITRs). In one example, a vector genome contains, at a minimum, from 5′ to 3′, an AAV2 5′ ITR, a hDBT (and/or BCKDHA and/or BCKDHB) nucleic acid sequence encoding a functional hMSUD-E2 (hMSUD-E1A or hMSUD-E1B) and an AAV2 3′ ITR. However, ITRs from a different source AAV other than AAV2 may be selected. Further, other ITRs may be used. Further, the vector genome contains regulatory sequences which direct expression of the functional subunit protein.

The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (ΔITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the E1A, E1B or E2 coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used.

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV Dnase-resistant particle having an AAV protein capsid into which is packaged expression cassette flanked by AAV inverted terminal repeat sequences (ITRs) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10). These documents also describe other AAV which may be selected for generating AAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV7, AAV8, AAV9, AAVrh10, AAVhu37, AAVrh32.33, AAV8 bp, AAV7M8 and AAVAnc80, AAV1, AAV2, AAV3, AAV4, AAV5, AAV6, AAV7, AAV8, AAV9.47, AAV9(hu14), AAV10, AAV 11, AAV12, AAVrh8, AAVrh74, AAV-DJ8, AAV-DJ, AAVhu68, without limitation, See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10), and, WO 2005/033321, which are incorporated herein by reference. Other suitable AAVs may include, without limitation, AAVrh90 [PCT/US20/31273, filed Apr. 28, 2020], AAVrh91 [PCT/US20/30266, filed Apr. 28, 2020], AAVrh92, AAVrh93, AAVrh91.93 [PCT/US20/30281, filed Apr. 28, 2020], which are incorporated by reference herein. As used herein, relating to AAV, the term “variant” means any AAV sequence which is derived from a known AAV sequence, including those with a conservative amino acid replacement, and those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, Va.). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

As used herein, the terms “rAAV” and “artificial AAV” used interchangeably, mean, without limitation, a AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

As used herein, “AAV9 capsid” refers to the AAV9 as defined in PCT/US19/19804, which is incorporated herein by reference. “AAV1 capsid” refers to the AAV9 as defined in PCT/US19/19804, which is incorporated herein by reference. As used herein, “AAV8 capsid” refers to the AAV8 as defined in PCT/US19/19804, which is incorporated herein by reference. In certain embodiments, the AAV9 has the encoded amino acid sequence of (a) GenBank accession: AAS99264, I and/or (b) the amino acid sequence encoded by the nucleotide sequence of GenBank Accession: AY530579.1: (nt 1 . . . 2211). Some variation from this encoded sequence is encompassed by the present invention, which may include sequences having about 99% identity to the referenced amino acid sequence in GenBank accession: AAS99264 and U.S. Pat. No. 7,906,111 (also WO 2005/033321) (i.e., less than about 1% variation from the referenced sequence). Such AAV may include, e.g., natural isolates (e.g., hu68 (described in “Novel Adeno-associated virus (AAV) Clade F Vector and Uses Therefor”, WO 2018/160582), hu31 or hu32), or variants of AAV9 having amino acid substitutions, deletions or additions, e.g., including but not limited to amino acid substitutions selected from alternate residues “recruited” from the corresponding position in any other AAV capsid aligned with the AAV9 capsid; e.g., such as described in U.S. Pat. Nos. 9,102,949, 8,927,514, US2015/349911; WO 2016/049230A11; U.S. Pat. Nos. 9,623,120; 9,585,971. However, in other embodiments, other variants of AAV9, or AAV9 capsids having at least about 95% identity to the above-referenced sequences may be selected. See, e.g., US Published Patent Application No. 2015/0079038. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, the rAAV as described herein is a self-complementary AAV. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In certain embodiments, the rAAV described herein is nuclease-resistant. Such nuclease may be a single nuclease, or mixtures of nucleases, and may be endonucleases or exonucleases. A nuclease-resistant rAAV indicates that the AAV capsid has fully assembled and protects these packaged genomic sequences from degradation (digestion) during nuclease incubation steps designed to remove contaminating nucleic acids which may be present from the production process. In many instances, the rAAV described herein is dNase resistant.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr. 15; 20(R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95(6):1081-92; SAMI S. THAKUR, Production of Recombinant Adeno-associated viral vectors in yeast. Thesis presented to the Graduate School of the University of Florida, 2012; Kondratov O et al. Direct Head-to-Head Evaluation of Recombinant Adeno-associated Viral Vectors Manufactured in Human versus Insect Cells, Mol Ther. 2017 Aug. 10. pii: S1525-0016(17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28(1):15-22. doi: 10.1089/hgtb.2016.164.; Li L et al. Production and characterization of novel recombinant adeno-associated virus replicative-form genomes: a eukaryotic source of DNA for gene transfer. pLoS One. 2013 Aug. 1; 8(8):e69879. doi: 10.1371/journal.pone.0069879. Print 2013; Galibert L et al, Latest developments in the large-scale production of adeno-associated virus vectors in insect cells toward the treatment of neuromuscular diseases. J Invertebr Pathol. 2011 July; 107 Suppl:S80-93. doi: 10.1016/j.jip.2011.05.008; and Kotin R M, Large-scale recombinant adeno-associated virus production. Hum Mol Genet. 2011 Apr. 15; 20(R1):R2-6. doi: 10.1093/hmg/ddr141. Epub 2011 Apr. 29.

A two-step affinity chromatography purification at high salt concentration followed by anion exchange resin chromatography are used to purify the vector drug product and to remove empty capsids. These methods are described in more detail in WO 2017/160360 entitled “Scalable Purification Method for AAV9”, which is incorporated by reference herein. In brief, the method for separating rAAV9 particles having packaged genomic sequences from genome-deficient AAV9 intermediates involves subjecting a suspension comprising recombinant AAV9 viral particles and AAV 9 capsid intermediates to fast performance liquid chromatography, wherein the AAV9 viral particles and AAV9 intermediates are bound to a strong anion exchange resin equilibrated at a pH of 10.2, and subjected to a salt gradient while monitoring eluate for ultraviolet absorbance at about 260 and about 280. Although less optimal for rAAV9, the pH may be in the range of about 10.0 to 10.4. In this method, the AAV9 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

Conventional methods for characterization or quantification of rAAV are available to one of skill in the art. To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Viral. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO stain. In one embodiment, the concentration of AAV vector genomes (vg) in column fractions can be measured by quantitative real time PCR (Q-PCR). Samples are diluted and digested with dNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the dNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Methods for determining the ratio among vp1, vp2 and vp3 of capsid protein are also available. See, e.g., Vamseedhar Rayaprolu et al, Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

As used herein, the term “treatment” or “treating” refers to composition(s) and/or method(s) for the purposes of amelioration of one or more symptoms of MSUD, restoration of a desired function of E2, or improvement of a biomarker of disease. In some embodiments, the term “treatment” or “treating” is defined as encompassing administering to a subject one or more compositions described herein for the purposes indicated herein. “Treatment” can thus include one or more of reducing onset or progression of MSUD, preventing disease, reducing the severity of the disease symptoms, retarding their progression, removing the disease symptoms, delaying progression of disease, or increasing efficacy of therapy in a given subject.

It should be understood that the compositions in the rAAV described herein are intended to be applied to other compositions, regiments, aspects, embodiments and methods described across the Specification.

### 5. PHARMACEUTICAL COMPOSITION

In certain embodiments, a composition is designed to include a single stock of vectors encoding MSUD-E2 (e.g., rAAV.hDBT), a single stock of vectors comprising an MSUD-E1A (e.g., rAAV.BCKDHA) or a single stock of vectors encoding MSUD-E1B (e.g., rAAV.BCKDHB). In certain embodiments, a composition is designed to contain a mixture of two or more viral stocks encoding different MSUD subunit proteins (e.g., a viral stock encoding MSUD E1A and a viral stock MSUD E2). In certain embodiments, a vector is engineered to contain two or more of these coding sequences. In certain embodiments, these compositions may be delivered via different routes, which may include two or more separately formulated vector stocks and/or two or more separately formulated LNPs being co-administered. Various combination of different vector stocks encoding MSUD-hE2 (e.g., rAAV.hDBT), MSUD-hE1A (e.g., rAAV.BCKDHA) and/or MSUD-hE1b (e.g., rAAV.BCKDHB) and/or one or more different LNPs carrying the mRNA encoding MSUD-hE2, MSUD-hE1A, and/or MSUD-hE1B may be selected. One, two or all three of the vectors and/or one, two or all three of the mRNA may comprises the engineered nucleic acids as provided herein. In certain embodiments, a composition or regimen is designed to include a combination of different recombinant AAV stocks. In certain embodiments, a composition or regimen is designed to include LNP(s) carrying a nucleic acid sequence (e.g., mRNA) encoding MSUD-hE2, MSUD-hE1A and/or MSUD-hE1B.

In embodiments where separate vectors are combined in a single composition or co-administered, the ratio of vectors or other compositions (e.g., LNP-mRNA) may be such that there are equivalent amounts of transgene nucleic acid delivered (e.g., a 1:1 ratio, or a 1:1:1 ratio). This may be determined based on genome copies (GC) for a vector or by weight of the nucleic acid sequences. In other embodiments involving combination therapy, the ratio of vectors or other composition may be varied so that the coding sequences for one MSUD subunit protein is delivered in an amount in excess of one or both of the coding sequence for the other subunit protein(s).

These compositions may be delivered intravenously, or by any other suitable route, e.g., oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In one aspect, provided herein is a pharmaceutical composition comprising a single vector stock or combinations thereof as described herein in a suitable carrier, diluent, and/or other excipient (e.g., in a formulation buffer).

In one embodiment, the pharmaceutical composition is suitable for co-administering with a functional hMSUD-E2 protein or a protein comprising a functional hMSUD-E1A, or hMSUD-E1B. In one embodiment, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL. In one embodiment, provided is a pharmaceutical composition comprising a mRNA encapsulated in LNP (mRNA-LNP) as described herein in a formulation buffer. In one embodiment, mRNA-LNP is formulated to comprise a combination of nucleic acid sequences encoding hMSUD-E2, hMSUD-E1A, and hMSUDE1B. In one embodiment, mRNA-LNP is formulated at about 0.1 μg/mL to 10 mg/mL.

In one embodiment, the formulation further comprises a surfactant, preservative, excipients, and/or buffer dissolved in the aqueous suspending liquid. In one embodiment, the buffer is PBS. Suitably, the formulation is adjusted to a physiologically acceptable pH, e.g., in the range of pH 6 to 8; for intravenous delivery, a pH of 6.8 to about 7.2 may be desired. However, other pHs within the broadest ranges and these subranges may be selected for other route of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Caprylocaproyl macrogol glycerides), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

Additionally provided is a pharmaceutical composition comprising a pharmaceutically acceptable carrier and a vector comprising a nucleic acid sequence encoding a functional E2 as described herein. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Delivery vehicles such as liposomes, nanocapsules, microparticles, microspheres, lipid particles, vesicles, and the like, may be used for the introduction of the compositions of the present invention into suitable host cells. In particular, the rAAV vector may be formulated for delivery either encapsulated in a lipid particle, a liposome, a vesicle, a nanosphere, or a nanoparticle or the like. In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

Also, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10⁵, 5×10¹⁵, 6×10¹⁵, 7×10¹⁵, 8×10¹⁵, or 9×10⁵ GC per dose including all integers or fractional amounts within the range. In one embodiment, for human application the dose can range from 1×10¹⁰ to about 1×10¹² GC per dose including all integers or fractional amounts within the range.

In one embodiment, the pharmaceutical composition comprising a rAAV as described herein is administrable at a dose of about 1×10⁹ GC per gram of brain mass to about 1×10¹⁴ GC per gram of brain mass.

In one embodiment, the pharmaceutical composition comprising mRNA-LNP as described herein is administrable at a dose of about 0.01, 0.03, 0.25, 0.6, 1, or 2 mg/kg. In one embodiment, mRNA-LNP is dosed weekly. In one embodiment, mRNA-LNP is dosed twice a week. In one embodiment, mRNA-LNP is dosed biweekly.

The aqueous suspension or pharmaceutical compositions described herein are designed for delivery to subjects in need thereof by any suitable route or a combination of different routes. In one embodiment, the pharmaceutical composition is formulated for delivery via intracerebroventricular (ICV), intrathecal (IT), or intracisternal injection. In one embodiment, the compositions described herein are designed for delivery to subjects in need thereof by intravenous injection. Alternatively, other routes of administration may be selected (e.g., oral, inhalation, intranasal, intratracheal, intraarterial, intraocular, intramuscular, and other parenteral routes).

It should be understood that the compositions in the pharmaceutical composition described herein are intended to be applied to other compositions, regiments, aspects, embodiments and methods described across the Specification.

### 6. METHOD OF TREATMENT

In one aspect, provided herein is a method of treating a human subject diagnosed with MSUD. The method comprises administering to a subject a suspension of a vector as described herein. In one embodiment, the method comprises administering to a subject a suspension of a rAAV as described herein in a formulation buffer.

The composition(s) and method(s) provided achieve efficacy in treating a subject in need with MSUD.

In certain embodiments, the human subject of these methods and compositions is a prenatal, a newborn, an infant, a toddler, a preschool, a grade-schooler, a teen, a young adult or an adult. In a further embodiment, the subject of these methods and compositions is a pediatric MSUD patient. In some embodiments, wherein a subject is prenatal, a newborn, an infant, a toddler, a preschool, a grade-schooler, a teen, a young adult, the subject received mRNA-LNP formulation, as described herein. In some embodiments, wherein a subject is an adult, the subject receives rAAV formulation, as described herein. In some embodiments, the mRNA-LNP formulation comprises a nucleic acid sequence encoding hMSUD-E2 and is used in treating a subject in need with MSUD. In some embodiments, the mRNA-LNP formulation comprises a nucleic acid sequence encoding hMSUD-E1A and is used in treating a subject in need with MSUD. In some embodiments, the mRNA-LNP formulation comprises a nucleic acid sequence encoding hMSUD-E1B and is used in treating a subject in need with MSUD. In certain embodiments, the mRNA-LNP formulation comprises a nucleic acid sequences encoding combination of hMSUD-E2, hMSUD-E1A, and/or hMSUD-E1B and is used in treating MSUD in a subject in need. In some embodiments, rAAV formulation comprises a nucleic acid sequence encoding hMSUD-E2 and is used in treating a subject with MSUD. In some embodiments, the rAAV formulation comprises a nucleic acid sequence encoding hMSUD-E1A and is used in treating a subject with MSUD. In some embodiments, the mRNA-LNP formulation comprises a nucleic acid sequence encoding hMSUD-E2 and is used in treating a subject with MSUD.

or a sequence identical thereto and/or combinations thereof.

In certain embodiments, one or more vectors (e.g., one or more viral (e.g., rAAV) or non-viral (e.g., LNP)) described herein deliver MSUD-E2, MDUD-E1A and/or MSUD-E1B to the muscle and liver. In certain embodiments, one or more vectors will target the muscle for expression of the subunit protein(s). In certain embodiments, one or vectors will target the liver for expression of the subunit protein(s). These vectors may be formulated separately or admixed and delivered together. In certain embodiments, the vector are formulated separately and delivered sequentially. For example, a patient may receive non-viral gene therapy (e.g., via an LNP, naked DNA, peptide, or liposomal delivery systems) at a younger age and then a viral vector—mediated gene therapy upon reaching a threshold age. In some embodiments, the patient may receive non-viral gene therapy through the age of 1 year, up through age 3, through age 12, through age 18. In certain embodiments, viral-mediated gene therapy may be administered to an infant, to a patient after 3 years of age, after 12 years of age, after 18 years of age, or at another suitable age.

As used herein, “facilitation of any treatment(s) for MSUD” or any grammatical variant thereof, refers to a decreased dosage or a lower frequency of a treatment of MSUD in a subject other than the composition(s) or method(s) which is/are firstly disclosed in the invention, compared to that of a standard treatment without administration of the described composition(s) and use of the described method(s).

An “increase in enzymatic activity” as used to reference MSUD-E2, E1A and/or E1B is used interchangeably with the term “increase in desired function”, and refers to activity at least about at least about 5%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of a healthy control. The enzymatic activity might be measured by any suitable assay.

In one embodiment, the suspension has a pH of about 7.28 to about 7.32.

In one embodiment, the subject is delivered a therapeutically effective amount of the vectors described herein. As used herein, a “therapeutically effective amount” refers to the amount of the composition comprising the nucleic acid sequence encoding a functional E2 which delivers and expresses in the target cells an amount of enzyme sufficient to achieve efficacy. In one embodiment, the dosage of the vector is about 1×10⁹ GC to about 1×10¹³ genome copies (GC) per dose. In specific embodiments, the dose of the vector administered to a patient is at least about 1.0×10⁹ GC/kg, about 1.5×10⁹ GC/kg, about 2.0×10⁹ GC/g, about 2.5×10⁹ GC/kg, about 3.0×10⁹ GC/kg, about 3.5×10⁹ GC/kg, about 4.0×10⁹ GC/kg, about 4.5×10⁹ GC/kg, about 5.0×10⁹ GC/kg, about 5.5×10⁹ GC/kg, about 6.0×10⁹ GC/kg, about 6.5×10⁹ GC/kg, about 7.0×10⁹ GC/kg, about 7.5×10⁹ GC/kg, about 8.0×10⁹ GC/kg, about 8.5×10⁹ GC/kg, about 9.0×10⁹ GC/kg, about 9.5×10⁹ GC/kg, about 1.0×10¹⁰ GC/kg, about 1.5×10¹⁰ GC/kg, about 2.0×10¹⁰ GC/kg, about 2.5×10¹⁰ GC/kg, about 3.0×10¹⁰ GC/kg, about 3.5×10¹⁰ GC/kg, about 4.0×10¹⁰ GC/kg, about 4.5×10¹⁰ GC/kg, about 5.0×10¹⁰ GC/kg, about 5.5×10¹⁰ GC/kg, about 6.0×10¹⁰ GC/kg, about 6.5×10¹⁰ GC/kg, about 7.0×10¹⁰ GC/kg, about 7.5×10¹⁰ GC/kg, about 8.0×10¹⁰ GC/kg, about 8.5×10¹⁰ GC/kg, about 9.0×10¹⁰ GC/kg, about 9.5×10¹⁰ GC/kg, about 1.0×10¹¹ GC/kg, about 1.5×10¹¹ GC/kg, about 2.0×10¹¹ GC/kg, about 2.5×10¹¹ GC/kg, about 3.0×10¹¹ GC/kg, about 3.5×10¹¹ GC/kg, about 4.0×10¹¹ GC/kg, about 4.5×10¹¹ GC/kg, about 5.0×10¹¹ GC/kg, about 5.5×10¹¹ GC/kg, about 6.0×10¹¹ GC/kg, about 6.5×10¹¹ GC/kg, about 7.0×10¹¹ GC/kg, about 7.5×10¹¹ GC/kg, about 8.0×10¹¹ GC/kg, about 8.5×10¹¹ GC/kg, about 9.0×10¹¹ GC/kg, about 9.5×10¹¹ GC/kg, about 1.0×10¹² GC/kg, about 1.5×10¹² GC/kg, about 2.0×10¹² GC/kg, about 2.5×10¹² GC/kg, about 3.0×10¹² GC/kg, about 3.5×10¹² GC/kg, about 4.0×10¹² GC/kg, about 4.5×10¹² GC/kg, about 5.0×10¹² GC/kg, about 5.5×10¹² GC/kg, about 6.0×10¹² GC/kg, about 6.5×10¹² GC/kg, about 7.0×10¹² GC/kg, about 7.5×10¹² GC/kg, about 8.0×10¹² GC/kg, about 8.5×10¹² GC/kg, about 9.0×10¹² GC/kg, about 9.5×10¹² GC/kg, about 1.0×10¹³ GC/kg, about 1.5×10¹³ GC/kg, about 2.0×10¹³ GC/kg, about 2.5×10¹³ GC/kg, about 3.0×10¹³ GC/kg, about 3.5×10¹³ GC/kg, about 4.0×10¹³ GC/kg, about 4.5×10¹³ GC/kg, about 5.0×10¹³ GC/kg, about 5.5×10¹³ GC/kg, about 6.0×10¹³ GC/kg, about 6.5×10¹³ GC/kg, about 7.0×10¹³ GC/kg, about 7.5×10¹³ GC/kg, about 8.0×10¹³ GC/kg, about 8.5×10¹³ GC/kg, about 9.0×10¹³ GC/kg, about 9.5×10¹³ GC/kg, or about 1.0×10¹⁴ GC/kg.

In one embodiment, the dosage of the mRNA-LNP is administrable at about 0.01, 0.03, 0.25, 0.6, 1, or 2 mg/kg. In one embodiment, mRNA-LNP is dosed weekly. In one embodiment, mRNA-LNP is dosed twice a week. In one embodiment, mRNA-LNP is dosed biweekly.

In one embodiment, the method further comprises the subject receives an immunosuppressive co-therapy. Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent.

In certain embodiments, the immunosuppressive therapy may be started 0, 1, 2, 7, or more days prior to the gene therapy administration. Such therapy may involve co-administration of two or more drugs, the (e.g., prednisone, mycophenolic acid (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected. In one embodiment, the rAAV as described herein is administrated once to the subject in need. In another embodiment, the rAAV is administrated more than once to the subject in need.

It should be understood that the compositions in the method described herein are intended to be applied to other compositions, regiments, aspects, embodiments and methods described across the Specification.

### 7. KIT

In certain embodiments, a kit is provided which includes a concentrated vector suspended in a formulation (optionally frozen), optional dilution buffer, and devices and components required for intrathecal, intracerebroventricular or intracisternal administration. In another embodiment, the kit may additional or alternatively include components for intravenous delivery. In one embodiment, the kit provides sufficient buffer to allow for injection. Such buffer may allow for about a 1:1 to a 1:5 dilution of the concentrated vector, or more. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

It should be understood that the compositions in kit described herein are intended to be applied to other compositions, regiments, aspects, embodiments and methods described across the Specification.

### 8. EXAMPLES

These examples are provided for the purpose of illustration only and the invention should in no way be construed as being limited to these examples but rather should be construed to encompass any and all variations that become evident as a result of the teaching provided herein.

Based on the mutation responsible for the deficiency in BCKDH activity, patients with MSUD have a variety of disease phenotypes. Approximately 75% of patients have classic MSUD resulting from <2% BCKDH activity. Patients with classic MSUD typically appear normal at birth with the disorder presenting during the first week of life as poor feeding, lethargy, weight loss, encephalopathy, acidosis, stereotypic movements, coma, and respiratory failure. The BCKDH complex consists of three catalytic proteins, E1, E2, and E3. Most of the mutations described have been in the E2 subunit of BCKDH.

In certain embodiments, rAAV expressing the E2 subunit of BCKDH targeted to the liver may be utilized.

In other embodiments, an rAAV stock for expressing one or more of the E1, E2 or E3 subunits in skeletal muscle is utilized. In certain embodiments, a combination of rAAV stocks each for expressing at least one of the subunits in skeletal muscle is delivered.

In certain embodiments, rAAV expressing E2 are co-administered for delivery to muscle and liver. In certain embodiments, combinations of rAAV expressing E1, E2 and/or E3 are co-administered for muscle and/or liver therapy.

However, an alternative is to use the oxidative enzyme capacity of skeletal muscle for metabolism of BCAAs instead of or in addition to that of the liver. In humans, skeletal muscle is responsible for 60% of the oxidative enzyme capacity compared to an approximately 10% contribution from the liver.

The intermediate or hypomorphic MSUD mouse is deficient in the mouse E2 subunit of BCKDH but has low levels of expression of human E2, which is required to rescue the neonatal lethality seen in the classic mouse E2 knockout. Hypomorphic MSUD mice exhibit decreased survival beginning at weaning and display elevated BCAA levels reminiscent of MSUD patients. Here, we evaluated the efficacy of a muscle-directed gene therapy approach compared to one directed to the liver.

For evaluation of a muscle-specific approach, we expressed E2 from a muscle-specific promoter following intramuscular (IM) administration of an adeno-associated viral (AAV) vector. A liver-specific approach was evaluated by expression of E2 from a liver-specific promoter following intravenous (IV) administration of an AAV vector. For evaluation of a combined approach, expression of E2 was driven from a promoter that is active in both muscle and liver following IM or IV administration of an AAV vector. Following IM injection substantial vector will target the liver resulting in approximately equal transduction and expression from each organ. Following vector administration into weaned hypomorphic MSUD mice, mice were evaluated for survival, body weight, and serum BCAA levels.

Untreated hypomorphic MSUD mice do not survive past day 30 of the study with extremely low body weights and uncontrolled serum BCAA levels. Only mice administered with the highest dose of vector using the muscle-specific promoter either by IV or IM injection survived until the end of the study (day 91) with higher serum BCAAs than other groups with similar survival levels. Hypomorphic MSUD mice administered with the liver-directed approach (IV injection of a vector containing the liver-specific TBG promoter) survived until the end of the study, with increases in body weight that did not reach that of an age-matched wild type mouse and poorly controlled serum BCAA levels. Utilizing the CB7 promoter for expression of E2 from both muscle and liver following IM injection resulted in survival until the end of the study, increases in body weight to that of wild type mice of equivalent age, and serum BCAA levels similar to that of wild type mice. However, when the same vector with the CB7 promoter was administered IV, which would result in substantially reduced muscle expression, survival and body weight was much reduced.

While rodents are not the ideal animal model for this research approach as only 30% of the oxidative capacity comes from skeletal muscle, compared to 60% in monkeys and humans, we have shown that a combined muscle and liver gene therapy approach has enhanced therapeutic potential compared to a liver only directed approach.

### Example 1: MSUD Mouse Models

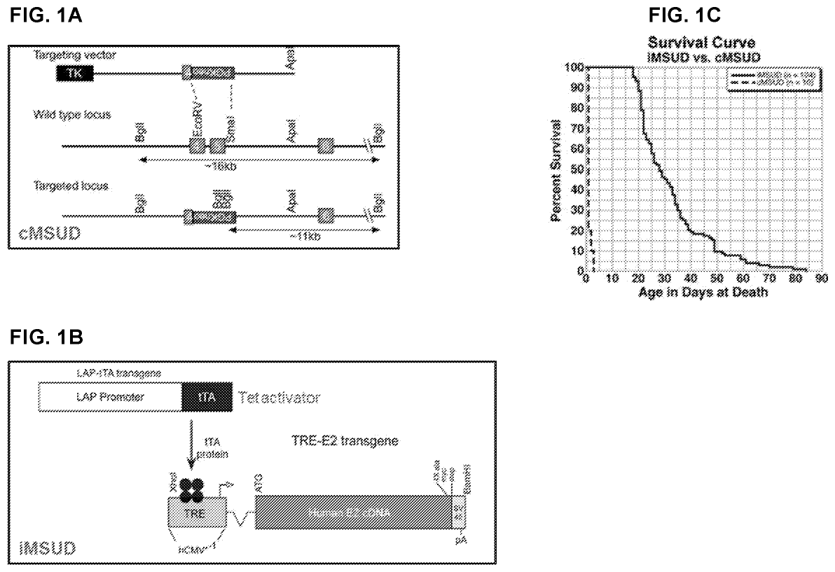

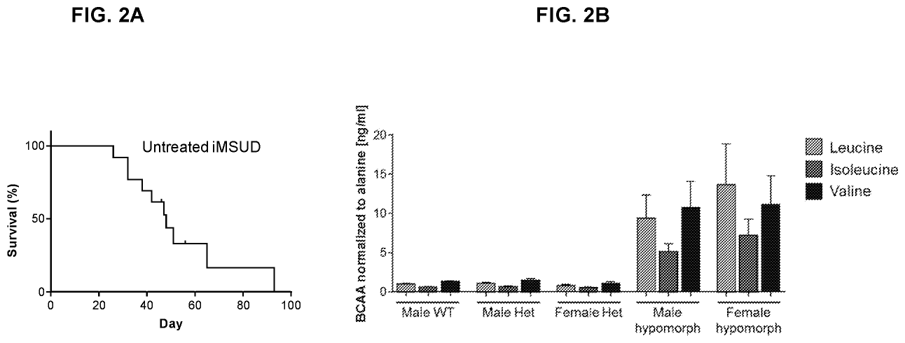

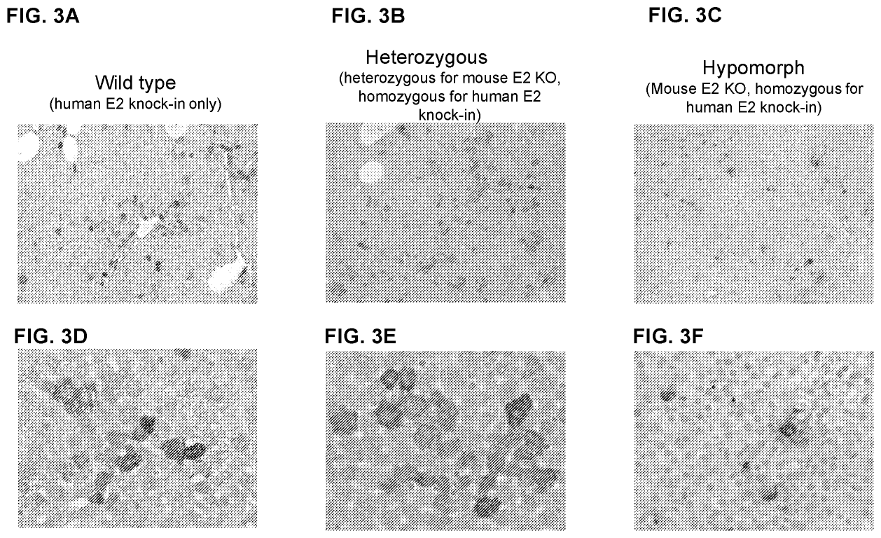

Natural history studies were performed in four MSUD mouse strains, these included two mouse models of MSUD that had been described: the classic (cMSUD) and intermediate (iMSUD) mouse models affecting the E2 subunit of BCKDH. FIG. 1A describes a cMSUD mouse model that was generated by Gregg E. Homanics at the University of Pittsburgh (Homanics G. E., et al., 2006, BMC Medical Genetics, 33). The cMSUD model has partial deletion of exon 4 and complete deletion of exon 5 in the mouse E2 subunit of BCKDH, which models the genotype that is most prevalent in human population. However, all cMSUD knockout (KO) animal die within 72 hours at birth, which requires early intervention. FIG. 1B describes an iMSUD mouse models for that was generated by Gregg E. Homanics at the University of Pittsburgh and donated to Jackson Laboratories (Homanics G. E., et al., 2006, BMC Medical Genetics, 33).). The iMSUD also has partial deletion of exon 4 and complete deletion of exon 5 in the mouse E2 subunit of BCKDH, but additionally has the human version of E2 knocked in, which corresponds to 5-6% of normal BCKDH activity. Hypomorph mice survive until early adulthood, but still have a reduced lifespan as compared to wild type mice. For iMSUD mice, when mouse E2 subunit of BCDKH is knocked out, a knock in of human version of E2 provides for a low expression, which is required to rescue the neonatal lethality seen in cMSUD KO (FIG. 1C). The iMSUD mice survive until early adulthood (FIG. 2A) and display elevated levels of BCAAs (FIG. 2B). For comparison, FIG. 3A to FIG. 3F shows anti-DBT (E2) immunohistochemistry (IHC) in wild type, heterozygous and polymorph mouse models.

Both, cMSUD and iMSUD mouse model has its advantages and disadvantages. For cMSUD, the advantage is that it provides for a KO mouse model with no interference from low level expression with human DBT/E2 protein. However, for cMSUD, the disadvantage is that the intervention needs to be administered immediately following birth. Additionally, there is a likely immune response to a non-self protein when DBT/E2 is expressed. For iMSUD mouse model, the advantage is that mice survive until weaning, so it is possible to model treatment in adolescents. Additionally, iMSUD mouse model provides immune tolerance to DBT protein as human version is present at low levels. However, for iMSUD, the disadvantage is that the mouse model is not a complete KO model due to presence of low-level expression of human DBT/E2 protein.

The majority of patients, approximately 80%, have mutations in the E2 subunit of BCDKH. Currently available mouse models, cMSUD and iMSUD, only model mutations in the DBT/E2 subunit of BCKDH. However, there are mutations with founder effects that have been seen in several populations including: Mennonite population of Pennsylvania and Costa Rican population. In Mennonite population of Pennsylvania, Y393N mutation is in BCKDHA/E1a subunit of BCKDH and has a prevalence of 1:176. This mutation corresponds to Y439N mutation in mouse sequence and can generFIate a classic phenotype model. In Costa Rican population, R285X mutation is in BCKDHB/E1β subunit of BCKDH, corresponds to R215X in the mouse sequence and generates a classic phenotype model.

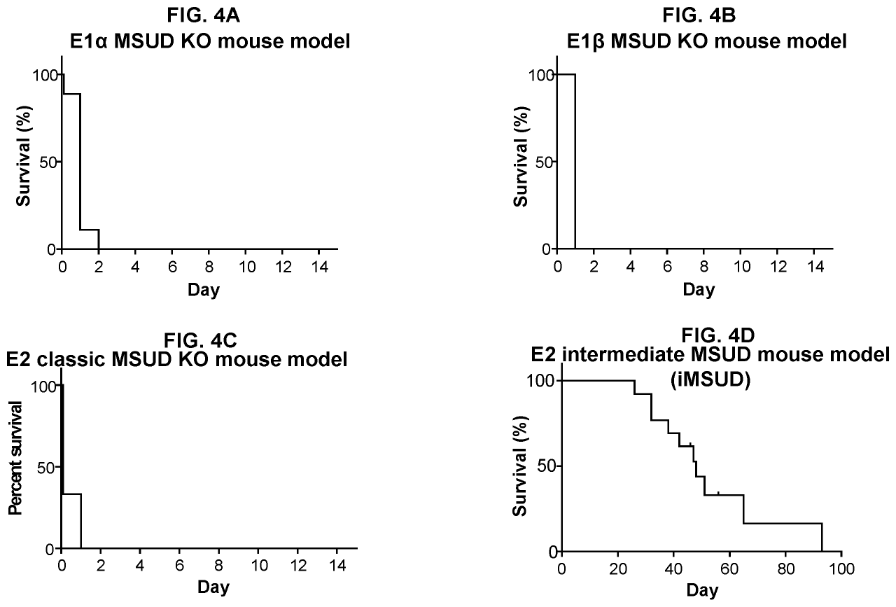

There is a need for new mouse models for MSUD to be developed, while reflecting mutations in E1α and E1β, including Type E1α Y393N mutation and Type E1β K241X mutation. We developed two new mouse strains to model founder effect mutations in the E1α and E1β subunits prevalent in the Mennonite and Costa Rican populations, respectively. Four MSUD mouse strains were evaluated for survival. The E1α (FIG. 4A), E1β (FIG. 4B), and E2 KO (FIG. 4C) mouse models all display the classical phenotype with survival only until day 1-2 of life. The iMSUD mice display the intermediate phenotype with survival past weaning till about 21 to about 28 day of life (FIG. 4D).

### Example 2: Methods Vector—AAV9.MSUD-E2

All AAV vectors were produced as previously described. Gao G, et al., Mol Ther. 2006; 13(1):77-87. rAAV9.MSUD-E2 (also termed rAAV9.MSUD.hDBTco), rAAV9-MSUD-E1A, and rAAV9-MSUD-E1B vectors are manufactured with iodixanol gradient method. See, Lock, M., et al., Rapid, Simple, and Versatile Manufacturing of Recombinant Adeno-Associated Viral Vectors at Scale. Human Gene Therapy, 2010. 21(10): p. 1259-1271. The purified vectors are titrated with classic qPCR.

A series of rAAV9.MSUD-E2 vectors are constructed. The rAAV.MSUD-E2 designed for muscle delivery include a muscle-specific promoter: a truncated MCK promoter [2R5-S(SEQ ID NO: 16) or SEQ ID NO: 17 or a constitutive promoter, CB7 [SEQ ID NO: 7]. The vector genomes generated are:

CB7.CI.hMSUD-E2.SV40, which includes the AAV2-5′ ITR, CB7 promoter, the CI Intron of SEQ ID NO: 8, the hMSUD-E2 [SEQ ID NO: 2], and the SV40 poly A [SEQ ID NO: 9], and the AAV2-3′ ITR.

tMCK.PI.hMSUD-E2co.SV40, which includes: the AAV2-5 ITR with a 22 bp deletion (SEQ ID NO: 15), two copies of the 2R5 S shortened MCK promoter (SEQ ID NO: 16), the PI intron (SEQ ID NO: 19), the hMSUD-E2 (SEQ ID NO: 2), the SV40 polyA, and the AAV 2-3′ ITR.

The rAAV.MSUD-E2 designed for liver delivery include a liver-specific promoter: TBG promoter with enhancer [SEQ ID NO: 18] or the constitutive CB7 promoter.

The vector genomes generated includes:
TBG.hMSUD-E2.WPRE.BGH which includes: the AAV2-5′ ITR, two copies of the alpha mic/bik (SEQ ID NO: 13), TBG promoter (SEQ ID NO: 10), the SV40 misc intron (SEQ ID NO: 14), WPRE (SEQ ID NO: 11), BGH poly A (SEQ ID NO: 12), and AAV 2-3′ ITR.
TBG.PI.hMSUD-E2.BGH includes the AAV2-5′ ITR, two copies of the alpha mic/bik (SEQ ID NO: 13), TBG promoter (SEQ ID NO: 10), the SV40 misc intron (SEQ ID NO: 14), WPRE (SEQ ID NO: 11), BGH poly A (SEQ ID NO: 12), and AAV 2-3′ ITR.

Using similar techniques, rAAV9 are constructed for E1A [SEQ ID NO: 3] and E1B [SEQ ID NO: 5]. One illustrative rAAV9 has a 5′ ITR, a TBG promoter with enhancers (two copies of alpha mic/bik) (SEQ ID NO: 18), an SV40 misc intron (SEQ ID NO: 14), hE1A coding sequence (SEQ ID NO: 4), a WPRE, a bGH poly A, and a 3′ ITR.

### Example 3. Muscle-Directed AAV Gene Therapy Rescues the Maple Syrup Urine Disease Phenotype in a Mouse Model

We evaluated the efficacy of a muscle directed gene therapy approach compared to one directed to the liver in a mouse model of MSUD. For these studies, we chose the intermediate MSUD (iMSUD) mouse model to evaluate our gene therapy approaches. [Homanics G E, et al, Production and characterization of murine models of classic and intermediate maple syrup urine disease. BMC Med Genet. 2006; 7:33; Skvorak K J. Animal models of maple syrup urine disease. J Inherit Metab Dis. 2009; 32(2):229-46]. The iMSUD mouse is a hypomorphic model, where the mouse DBT gene is knocked out (KO) by partial deletion of exon 4 and complete deletion of exon 5 and the human DBT gene is knocked in, expressing at 5-6% activity. The presence of low level expression from the human DBT gene allows these mice to survive until weaning, compared to DBT KO mice that survive for less than 72 hours. Skvorak (2009), cited above. While the distribution of BCKDH activity in rats is different from that in humans with liver providing 60-83% activity and only 3-29% activity from skeletal muscle (Suryawan A, et al., A molecular model of human branched-chain amino acid metabolism. Am J Clin Nutr. 1998; 68(1):72-81) the ability to improve the MSUD phenotype in a mouse model may provide insight into the feasibility of a gene therapy approach for patients with MSUD.

Systemically delivering an AAV vector to the liver that expressed DBT from the liver-specific TBG promoter did not sufficiently ameliorate all aspects of the disease phenotype in the iMSUD mouse. A similar effect occurred when we used the ubiquitous CB7 promoter for expression following intravenous vector administration. These findings underscored the need for an alternative approach. In humans, muscle is a larger source of BCAA metabolism than liver. However, when we deployed a muscle-specific approach using the tMCK promoter for expression of DBT following intramuscular (IM) administration, this strategy only partially rescued the MSUD phenotype. When we combined the ubiquitous CB7 promoter with vector delivery by IM injection, survival drastically increased across all assessed doses. Additionally, serum BCAA levels remained near normal levels in the mid- and high-dose cohorts throughout the study duration. This gene therapy approach also protected iMSUD mice from lethal high protein diet challenge. Therefore, administering a gene therapy vector that expresses in both the muscle and liver may be a viable alternative treatment path for patients with MSUD. Table 1 and Table 2 provides a summary of gene therapy efficacy following administration to the hypomorphic iMUSD mouse model.

TABLE 1
Gene therapy efficacy in iMSUD mouse model.

| | | | | Doses | | Survival |
|---|---|---|---|---|---|---|
| Name | ROA | Capsid | Promoter | (GC/kg) | n | (%) |
| Untreated | N/A | N/A | N/A | N/A | 7 | 0% |
| Highly muscle-specific | IM | AAV9 | tMCK | 3.00E+09 | 2 | 50% |
| approach | | | | | | |
| Highly muscle-specific | IM | AAV9 | tMCK | 3.00E+11 | 4 | 0% |
| approach | | | | | | |
| Highly muscle-specific | IM | AAV9 | tMCK | 3.00E+12 | 5 | 0% |
| approach | | | | | | |
| Highly muscle-specific | IM | AAV9 | tMCK | 3.00E+13 | 7 | 71% |
| approach | | | | | | |
| Muscle-specific | IV | AAV9 | tMCK | 3.00E+11 | 3 | 33% |
| approach | | | | | | |
| Muscle-specific | IV | AAV9 | tMCK | 3.00E+12 | 5 | 40% |
| approach | | | | | | |
| Muscle-specific | IV | AAV9 | tMCK | 3.00E+13 | 4 | 100% |
| approach | | | | | | |
| Liver-specific approach | IV | AAV8 | TBG | 3.00E+13 | 5 | 100% |
| Muscle injected | IM | AAV9 | CB7 | 3.00E+09 | 3 | 33% |
| combined approach | | | | | | |
| Muscle injected | IM | AAV9 | CB7 | 3.00E+11 | 6 | 100% |
| combined approach | | | | | | |
| Muscle injected | IM | AAV9 | CB7 | 3.00E+12 | 6 | 100% |
| combined approach | | | | | | |
| Muscle injected | IM | AAV9 | CB7 | 3.00E+13 | 5 | 80% |
| combined approach | | | | | | |
| Systemic injected | IV | AAV9 | CB7 | 3.00E+11 | 3 | 67% |
| combined approach | | | | | | |
| Systemic injected | IV | AAV9 | CB7 | 3.00E+12 | 5 | 80% |
| combined approach | | | | | | |
| Systemic injected | IV | AAV9 | CB7 | 3.00E+13 | 3 | 33% |
| combined approach | | | | | | |

TABLE 2
Further view of the efficacy of gene therapy in
iMSUD mouse model (continuation of Table 1).

| | | End of study | | End of study | |
|---|---|---|---|---|---|
| | | average | | average | |
| | Survival | body | Body weight | leucine | Leucine |
| Name | advantage | weight (g) | improvement | (ng/ml) | improvement |
| Untreated | No | 8.80 | No | 501600 | No |
| Highly muscle-specific | No | 22.10 | No | 90800 | No |
| approach | | | | | |
| Highly muscle-specific | No | 8.65 | No | 147150 | No |
| approach | | | | | |
| Highly muscle-specific | No | 10.50 | No | 141333 | No |
| approach | | | | | |
| Highly muscle-specific | Partial | 20.80 | Corrected | 51425 | Partial |
| approach | | | | | |
| Muscle-specific approach | Partial | 16.70 | Partial | 172000 | No |
| Muscle-specific approach | Partial | 17.00 | Partial | 130600 | No |
| Muscle-specific approach | Yes | 22.93 | Corrected | 37467 | Partial |
| Liver-specific approach | Yes | 17.30 | Partial | 143800 | Partial |
| Muscle injected combined | No | 17.50 | No | 209000 | No |
| approach | | | | | |
| Muscle injected combined | Yes | 22.58 | Corrected | 119820 | No |
| approach | | | | | |
| Muscle injected combined | Yes | 21.60 | Corrected | 59000 | Partial |
| approach | | | | | |
| Muscle injected combined | Partial | 15.77 | Partial | 21433 | Corrected |
| approach | | | | | |
| Systemic injected combined | Partial | 19.75 | Corrected | 137500 | No |
| approach | | | | | |
| Systemic injected combined | Partial | 20.16 | Corrected | 55000 | Partial |
| approach | | | | | |
| Systemic injected combined | Partial | 16.90 | Partial | 24600 | Corrected |
| approach | | | | | |

### A. Materials and Methods

AAV Vector Production: All AAV vectors were produced as previously described. Gao G, et al., Mol Ther. 2006; 13(1):77-87. Briefly, plasmids expressing a codon-optimized version of human DBT (hDBTco) from the thyroxine binding globulin (TBG) promoter were packaged within the AAV8 capsid, and plasmids expressing hDBTco from either the chicken beta actin (CB7) promoter or muscle creatinine kinase (tMCK) promoter were packaged within the AAV9 capsid. See, Example 1 above.

Mice: Breeding pairs of heterozygous Dbt^{tm1}Geh Tg(Cebpb-tTA)5Bjd Tg(tetO-DBT)A1Geh/J mice were obtained from The Jackson Laboratory (Bar Harbor, Me.), hereafter to be referred to as iMSUD. A colony was maintained at the University of Pennsylvania under specific pathogen-free conditions. All animal procedures and protocols were approved by the Institutional Animal Care and Use Committee of the University of Pennsylvania. P21-28 iMSUD mice received an IV injection with 3×10¹³ genome copies (GC)/kg of AAV8.TBG.hDBTco, 3×10¹¹-3×10¹³ GC/kg of AAV9.CB7.hDBTco, or 3×10¹²-3×10¹³ GC/kg of AAV9.tMCK.hDBTco via the retro-orbital or tail vein depending on the size of the mouse at the time of injection. Additional iMSUD mice received an IM injection with 3×10¹¹-3×10¹³ GC/kg of AAV9.CB7.hDBTco into the gastrocnemius muscles of both hind limbs. Mice were monitored for survival and changes in body weight throughout the in-life phase of the study.

High protein diet challenge: An additional cohort of iMSUD mice were administered IM with one of three doses of AAV9.CB7.hDBTco vector in the same manner as described previously were challenged with a high protein diet. Groups of untreated mice were included as a negative control (untreated iMSUD, heterozygous, and wild type mice). Fourteen days post-vector administration, mice challenged with a high protein diet for seven days.

Serum analyses: Blood was collected in serum separator tubes and allowed to clot. Serum was isolated and analyzed for leucine levels by Charles River Laboratories (Wilmington, Mass.).

In situ hybridization: Liver samples were fixed in 10% neutral buffered formalin and used for determination of hUGT1A1co messenger RNA expression by ISH, as described previously. [Hinderer C, et al. Severe Toxicity in Nonhuman Primates and Piglets Following High-Dose Intravenous Administration of an Adeno-Associated Virus Vector Expressing Human SMN. Hum Gene Ther. 2018.] Z-shaped probe pairs specific for hDBTco were synthesized by the kit manufacturer. Sections were counterstained with DAPI to show nuclei.

Vector genome copy and transgene RNA analysis: Liver and muscle samples were snap frozen at the time of necropsy, and DNA was extracted using the QIAamp DNA Mini Kit (Qiagen, Valencia, Calif.). dNase treated total RNA was isolated from 100 mg of tissue. RNA was quantified by spectrophotometry and aliquots reverse transcribed to cDNA using random primers. Detection and quantification of vector GC in extracted DNA and relative hDBTco transcript expression in extracted RNA were performed by real-time PCR, as described previously. [Greig J A, et al. Intramuscular injection of AAV8 in mice and macaques is associated with substantial hepatic targeting and transgene expression. pLoS One. 2014; 9(11):e112268; Bell P, et al. Analysis of tumors arising in male B6C3F1 mice with and without AAV vector delivery to liver. Mol Ther. 2006; 14(1):34-44]. Briefly, vector GC and RNA levels were quantified using primers/probe designed against a transgene-specific sequence.

Statistical analysis: All values are presented as mean±SEM. Treatment groups were compared using a one-way ANOVA with Tukey's multiple comparison test. A p value of <0.05 was considered significant.

### B. Results

Limited Effectiveness of Systemically Administered Gene Therapy Approach

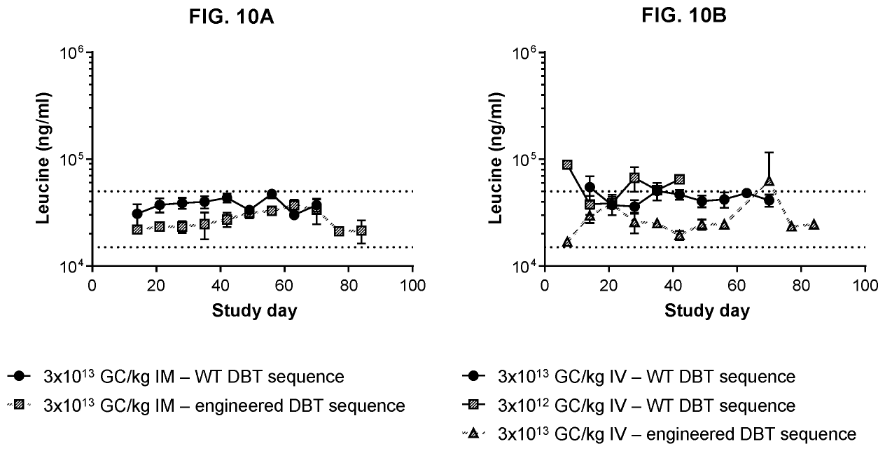

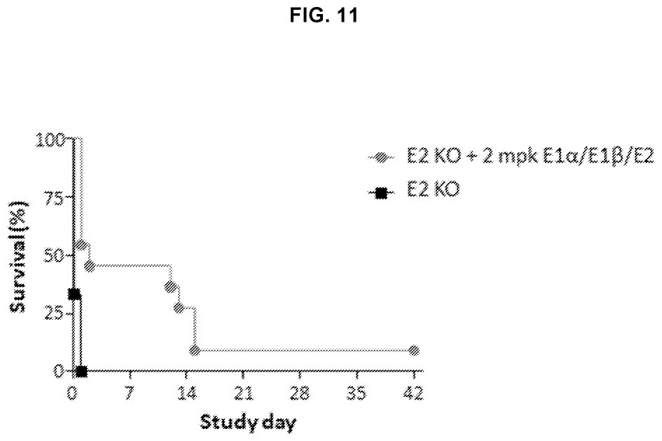

As the current standard of care for MSUD is a restricted diet or liver transplant, we initially attempted a traditional liver-directed gene therapy approach using the liver-specific TBG promoter for expression following IV injection. As the iMSUD mice are a hypomorphic model of DBT deficiency, the vector expressed an engineered version of human dihydrolipoamide branched-chain transacylase (hDBTco). For comparison, FIG. 10 shows serum leucine levels in iMSUD mice following injection via intravenously (FIG. 10B) or intramuscularly (FIG. 10A) with an AAV9.CB7 viral particles at doses of 3×10¹² and 3×10¹³ GC/kg, as indicated, comprising a wild-type (WT) or engineered DBT/E2 nucleic acid sequence encoding human E2 protein.

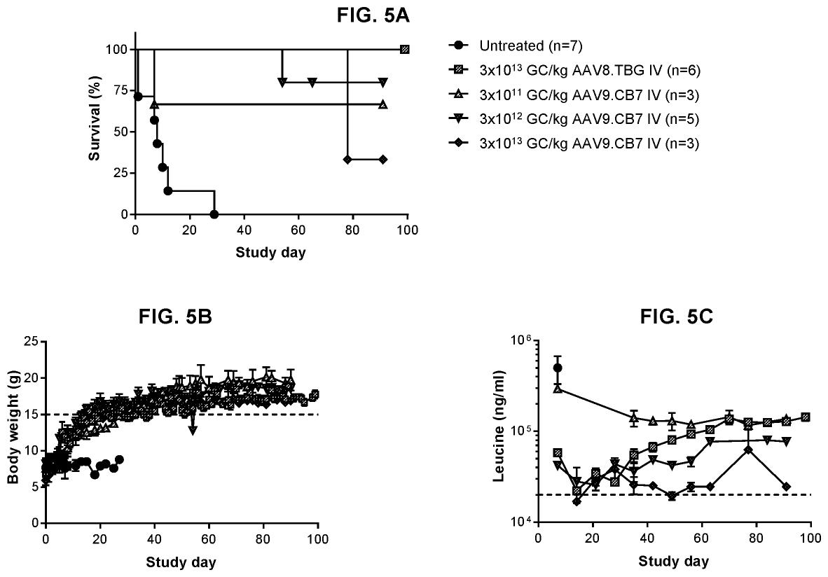

iMSUD mice were administered with 3×10¹³ GC/kg of AAV8.TBG.hDBTco on P21-28 and monitored for survival, body weight, and serum leucine levels throughout the in-life phase of the 91-day study. Additional mice were administered with 3×10¹¹, 3×10¹², or 3×10¹³ GC/kg of AAV9.CB7.hDBTco to evaluate expression from an alternative highly expressed promoter. We compared all parameters to a group of untreated iMSUD mice (FIGS. 5A to 5C).

Natural history studies performed by us and others (Homanics (2006); Skvorak (2009)) have found that iMSUD mice do not survive past 100 days of life. At the time of weaning (P21-28), iMSUD mice have reduced body weights compared to heterozygous and wild type littermates with the iMSUD mice enrolled into our studies averaging 7.9 g in body weight. In our current study, untreated iMSUD mice did not survive past study day 29, failed to gain weight over this time, and had average serum leucine levels of 501,600 ng/ml (FIGS. 5A to 5C).

In comparison to untreated controls, all mice treated with the AAV8.TBG.hDBTco vector survived until the end of the study (FIG. 5A). These mice gained weight rapidly following vector administration and initially had significantly reduced serum leucine levels (FIGS. 5B and 5C). However, over the course of the study leucine levels rose from a trough of 22,200 ng/ml (close to wild type levels) to a plateau of 132,206 ng/ml from day 70 onwards (FIG. 5C), indicating a waning in efficacy from the gene therapy vector. Following IV administration of a vector using the CB7 promoter for expression, the majority of gene expression will come from the liver in mice, even though the promoter is active in other cell types. (Grieg 2014). We were unsurprised to see a similar waning effect on serum leucine levels was seen for mice administered with the middle and high dose of AAV9.CB7.hDBTco (FIG. 5C), but mice administered with this vector also had reduced survival compared to the AAV8.TBG.hDBTco administered group (FIG. 5A), while increases in body weight were similar (FIG. 5B). The low dose of AAV9.CB7.hDBTco resulted in 67% survival, only minor changes in serum leucine levels, but interestingly the same increases in body weight as for the other vector treated groups (FIGS. 5A to 5C). Therefore, the limited effectiveness of a liver-directed approach suggested that an alternative gene therapy approach would be required for complete reversal of the MSUD phenotype in this mouse model.

Vast Improvement in Body Weight Following a Muscle-Specific Gene Therapy Approach

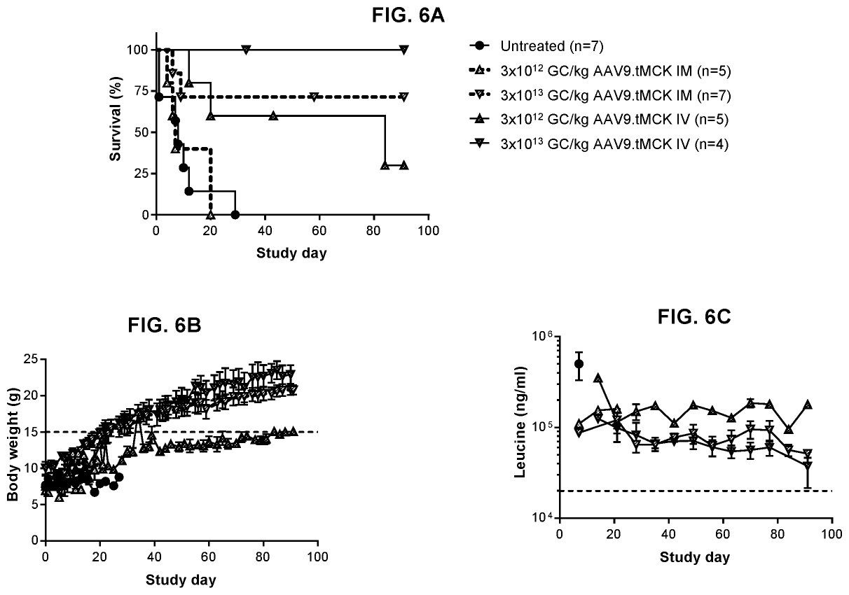

We attempted a muscle-specific gene therapy approach in the iMSUD mice using the muscle-specific tMCK promoter for expression. Mice were administered with 3×10¹² or 3×10¹³ GC/kg of AAV9.tMCK.hDBTco by either IM or IV injection, and compared to the same group of untreated iMSUD as previously described above (FIGS. 6A to 6C).

3×10¹² GC/kg of AAV9.tMCK.hDBTco administered IM did not improve survival compared to untreated iMSUD mice and there was no increase in body weight seen in these animals (FIGS. 6A and 6B). Whereas, the same vector dose administered IV did improve survival with only a minor improvement in body weight. For the high dose vector administered animals, both routes of administration enhanced survival and vastly increased body weight with mice averaging 20.8 g and 22.9 g in body weight by the end of the study for IM and IV vector delivery, respectively (FIGS. 6A and 6B). Interesting, administration of AAV9.tMCK.hDBTco by either route of administration at either dose resulted in similar limited reductions in serum leucine levels compared to the control group (FIG. 6C). Due to the inability to normalize serum leucine levels even at a high vector dose, a muscle-specific approach does not appear to be the way forward for treatment of MSUD.

Dose-Dependent Improvement in iMSUD Phenotype with a Combined Muscle and Liver Gene Therapy Approach

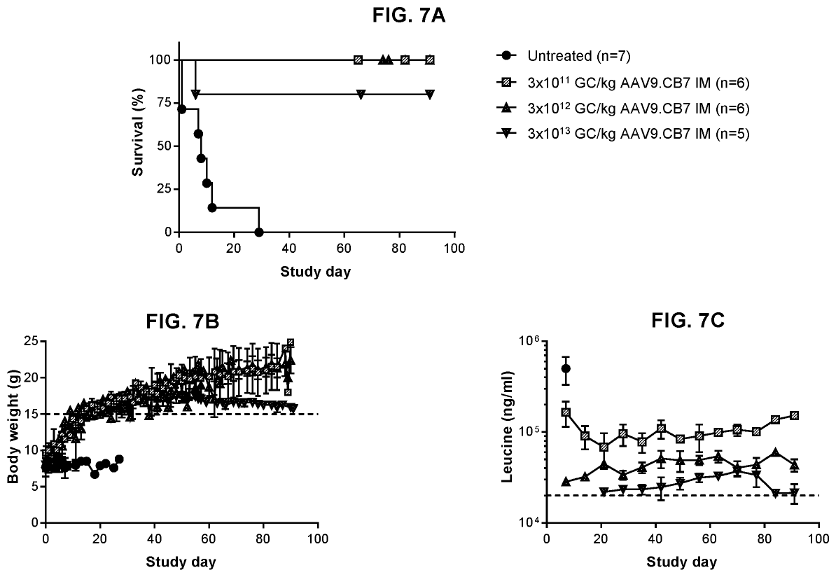

We evaluated the ability of a combined muscle and liver directed approach following IM administration of 3×10¹¹-3×10¹³ GC/kg of AAV9.CB7.hDBTco in iMSUD mice using the same untreated controls for comparison (FIGS. 7A to 7C). Low, middle, and high dose treated mice demonstrated enhanced survival compared to the control iMSUD mice, with mice administered with 3×10¹¹ and 3×10¹² GC/kg all surviving until the end of the study (FIG. 7A). Vast improvements in body weight were seen following vector administration, with the low and middle doses of vector appearing to outperform the high dose with final body weights of 21.5 g and 22.4 g, respectively (FIG. 7B). There was a dose-dependent reduction in serum leucine levels that was maintained throughout the in-life phase of the study, and mice administered with 3×10¹³ GC/kg of AAV9.CB7.hDBTco had approximately normalized leucine levels (wild type levels are approximately 20,000 ng/ml, indicated by the dotted line in FIG. 7C).

Improved Efficacy Resulting from High Gene Expression in Muscle

At the end of the study, mice were necropsied with liver and muscle harvested for evaluation of hDBTco RNA expression by ISH. As the iMSUD mouse model has reportedly 5-6% expression of human DBT (Homanics (2006); Skvorak (2009)) we designed the probes used for the ISH to bind only to the codon-optimized hDBTco sequence. As expected, there was no hDBTco RNA detected in untreated iMSUD mice as determined by the lack staining by ISH (data not shown). Only following high dose (3×10¹³ GC/kg) IM injection of AAV9.tMCK.hDBTco was hDBTco RNA expression detected by ISH in the injected muscle with some positive hepatocytes in the liver. Both the middle (3×10¹² GC/kg) and high (3×10¹³ GC/kg) doses of AAV9.CB7.hDBTco resulted in ISH detectable RNA in the injected muscle and liver, with mice administered with the high dose showing hDBTco RNA expression in uninjected muscle. Therefore, the improved efficacy seen with the combined muscle and liver approach following IM injection of AAV9.CB7.hDBTco was high expression from the CB7 promoter across both organ systems, including the ability of the vector to transduce uninjected skeletal muscle. An observed enhanced transgene expression in both liver and muscle with the CB7 promoter in the intermediate mouse model of MSUD supports the results as described above.

Enhanced RNA Expression Per Vector Genome Copy from the CB7 Promoter

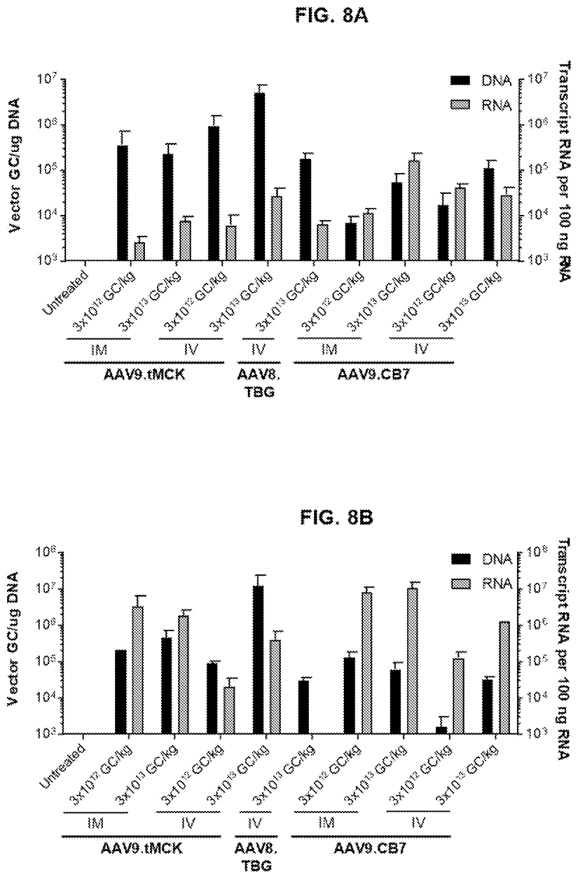

To further evaluate the enhanced efficacy observed following treatment of iMSUD with the AAV9.CB7.hDBTco vector by IM injection, we determined the vector GC and hDBTco RNA transcript levels in liver and injected muscle (FIGS. 8A and 8B). Following systemic administration of the AAV8.TBG.hDBTco vector, vector GCs were detected in both the liver and muscle (X-fold lower vector GC in muscle), but hDBTco RNA transcript levels were only detected in the liver due to the tissue specificity of the TBG promoter (FIGS. 8A and 8B).

Vector GCs detected in the liver following either IM or IV administration of AAV9.tMCK.hDBTco were, on average, 35-fold higher than those in mice administered with AAV9.CB7.hDBTco by either route (FIG. 8A). Following either IM or IV injection similar vector GCs were detected in muscle for the AAV9 vectors, reflective of the ability of this capsid to transduce muscle (FIG. 8B). For evaluation of the hDBTco RNA levels in both liver and muscle, and comparison to the previously described physiological data collected, the efficacy of these vectors at normalizing serum leucine levels is likely linked to their ability to express hDBTco from the vector genome in a given organ. AAV9.CB7.hDBTco appears to be the most efficient vector with the highest relative RNA levels per vector GC in either liver or muscle (FIGS. 8A and 8B).

Enhanced Survival in Gene Therapy Treated iMSUD Mice in Response to Challenge with a High Protein Diet

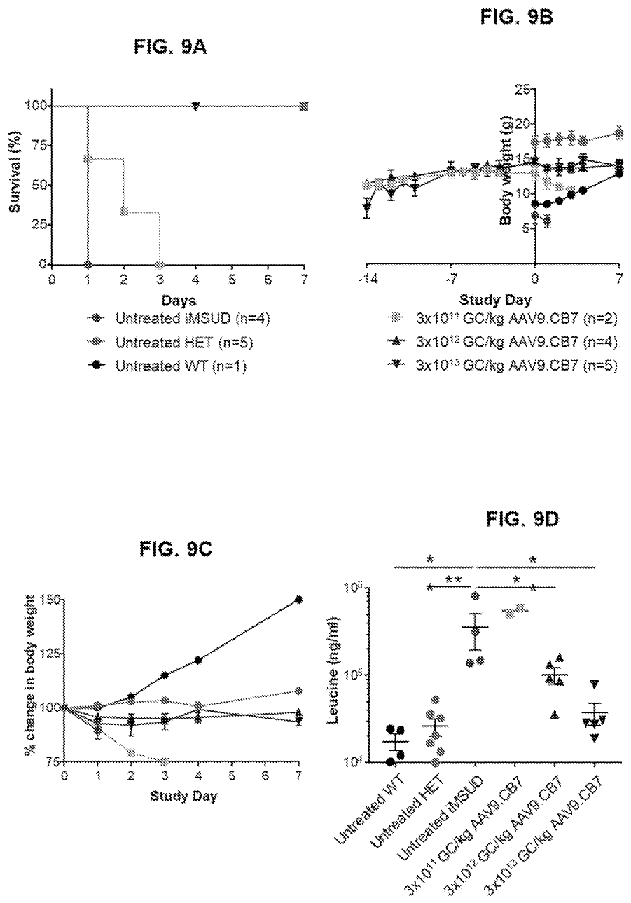

Patients with MSUD need to be on a restricted low protein diet in an effort to control their BCAA levels. Within hours following liver transplant, MSUD patients have decreased BCAA levels, their leucine tolerance levels are increased ten-fold, and they can eat a normal diet. Mazariegos (2012), cited above. Therefore, to provide functional evidence of efficacy with the combined muscle and liver gene therapy approach, we attempted to model the clinical leucine tolerance test by providing the mice with a high protein diet. An additional group of iMSUD mice that received AAV9.CB7.hDBTco by IM injection were challenged with a high protein diet for seven days (FIGS. 9A to 9D). Vector administered mice were followed for 14 days post-IM injection to allow hDBTco transgene expression levels to peak prior to high protein diet challenge, whereas control groups were initiated on the same diet on study day 0. Wild type and heterozygous littermates of the iMSUD mice used in this study were not affected by the high protein diet as there was no change in survival or serum leucine levels (FIGS. 9A and 9D). Untreated iMSUD mice succumbed to the high protein diet challenge within one day following a drop in body weight (FIGS. 9A and 9C). Mice injected with the low dose (3×10¹¹ GC/kg) of AAV9.CB7.hDBTco survived for up to three days on the high protein diet with substantial weight loss and no change in serum leucine levels compared to untreated iMSUD mice at the time of death. iMSUD mice administered with the middle and high dose of AAV9.CB7.hDBTco survived for the duration of the seven day challenge with no change in body weight and a vector dose-dependent decrease in serum leucine levels, indicating that this gene therapy approach not only improves the MSUD phenotype in this mouse model, but also protects survival in extreme situations (FIGS. 9A to 9D).

C. Discussion

Here, we evaluated the potential of liver and muscle directed gene therapy applications for MSUD by utilizing the iMSUD mouse model. Systemic administration of AAV vectors to the liver, which express hDBTco from the liver-specific TBG promoter, resulted in an initial correction of serum leucine levels to approximately wild type levels, but the efficacy waned over time and did not sufficiently ameliorate all aspects of the disease phenotype in the iMSUD mouse. A similar effect occurred when we used the ubiquitous CB7 promoter for expression following IV vector administration. The decrease in efficacy of liver-directed gene therapy over time is either due to growth of the animal (as seen by the increase in body weight) and resulting increase in liver size, [Wang L, et al, Hepatic gene transfer in neonatal mice by adeno-associated virus serotype 8 vector. Hum Gene Ther. 2012; 23(5):533-9] or due to a reduction in activity of a liver-directed gene therapy approach over time as seen for other disease applications.

In humans, muscle is a much larger source of BCKDH activity for BCAA metabolism than the liver. Suryawan (1998), cited above. However, when we deployed a muscle-specific approach using the tMCK promoter for expression of DBT following intramuscular (IM) administration, this strategy only partially rescued the MSUD phenotype. This could be partly due to differences in BCKDH activity across organs in mice (inferred by data in rats) and humans. In rats, 60-83% BCKDH activity is derived from the liver compared to 9-13% in humans. Suryawan (1998), cited above. Therefore, replacement of the deficient DBT gene in the skeletal muscle only in this mouse model may not improve the overall oxidative capacity for BCAA metabolism in this mouse model. When we combined the ubiquitous CB7 promoter with vector delivery by IM injection, survival drastically increased across all assessed doses. Additionally, serum BCAA levels remained near normal levels in the mid- and high-dose cohorts throughout the study duration. We have previously demonstrated that the CB7 promoter will express equally in muscle and liver of mice following IM injection. Grieg (2014), cited above. Here, we also found that AAV9.CB7.hDBTco was able to transduce uninjected skeletal muscle, increasing total body expression of DBT.

We have previously shown that the CB7 promoter is less active in the liver of primates, than it is in mice. Grieg (2014), cited above. However, due to the differences in location of BCKDH activity between rats and humans, a muscle-directed approach can result in correction of BCKDH activity. Direct injection into skeletal muscle will vastly expand the number of participants eligible for gene therapy. Systemic gene therapy into the vasculature requires strict inclusion criteria based on the pre-existing neutralizing antibody titers to the vector capsid, as titers ≥1/10 results in ablation of transgene expression in the liver. [Wang L, et al. Hum Gene Ther. 2011; 22(11):1389-401.] Titers up to 1/160 have been previously shown to have no impact on transgene expression from skeletal muscle [Greig J A, et al. Vaccine. 2016; 34(50):6323-9], but may impact on expression from liver or uninjected muscle groups.

In an effort to model an extreme situation that should result in metabolic crisis in the iMSUD mice, we provided a high protein diet challenge for seven days. IM administration of AAV9.CB7.hDBTco at doses ≥3×10¹² GC/kg protected iMSUD mice from the lethal high protein challenge and retained serum leucine levels close to normal. Therefore, if transgene expression levels following gene therapy is sufficient patients should be protected from metabolic crisis.

While this gene therapy approach was developed in the iMSUD mouse model, which is a hypomorphic model of DBT deficiency, this approach is applicable to mutations in the other genes that make up the BCKDH enzyme complex (BCKDHA and BCKDHB). Administering a gene therapy vector that expresses in both the muscle and liver may be a viable alternative treatment path for patients with MSUD.

### Example 4: Rescue Acute Crisis in Newborn Classic MSUD Mouse Models with Triple mRNA LNP

Lipid Nanoparticle mRNA Therapy Improves Survival in a Mouse Model of Classic Maple Syrup Urine Disease

Deficiency in the branched-chain alpha-keto acid dehydrogenase (BCKDH) enzyme complex results in a rare metabolic disorder called maple syrup urine disease (MSUD). Similar to other metabolic diseases, MSUD can range in severity. The neonatal onset seen in the classic form of MSUD is the most severe form of the disease. Current treatment options for MSUD are limited, and patients must typically follow a carefully monitored, restricted diet, with the potential for undergoing liver transplantation.

Based on our previous success in using lipid nanoparticle (LNP) mRNA therapy in a mouse model of a less severe, intermediate form of MSUD, we evaluated the same treatment approach in a mouse model of classic MSUD.

LNP Encapsulated mRNA Extended Survival

The classic MSUD mouse model is deficient in the mouse E2 subunit of BCKDH and demonstrates a lethal neonatal phenotype. E2 knockout (KO) mice do not survive past the second day of life. We evaluated the use of LNPs to deliver mRNA, wild type nucleic acid sequence, encoding three BCKDH subunits (E1a/E1b/E2) in E2 KO mice.

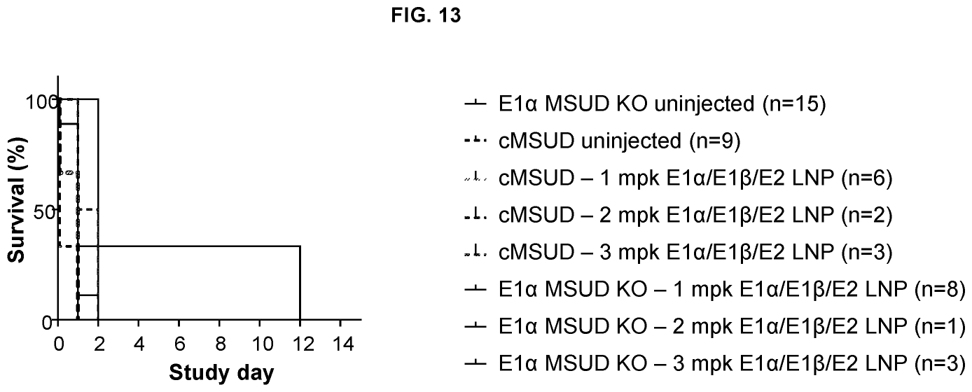

FIG. 13 shows an extended survival of classic MSUD mice following intravenous LNP encapsulated mRNA administration. We administered newborn mice with 2 mg per kg (mpk) of LNP encapsulated mRNA for the three BCKDH subunits (E1a/E1b/E2) intravenously (IV) on days 0 and 3 of life via the facial vein. Starting on day 7, mice received weekly or biweekly IV administrations of LNPs via either the retro-orbital or tail veins. Mice were followed for survival. Treatment with LNP encapsulated mRNA extended survival in this severe model of classic MSUD. Mean survival was extended to 5 days in a total of 22 treated E2 KO mice. Some mice survived to 11, 13, 15, and 40 days

LNP Encapsulated mRNA Reduced Serum Leucine Levels

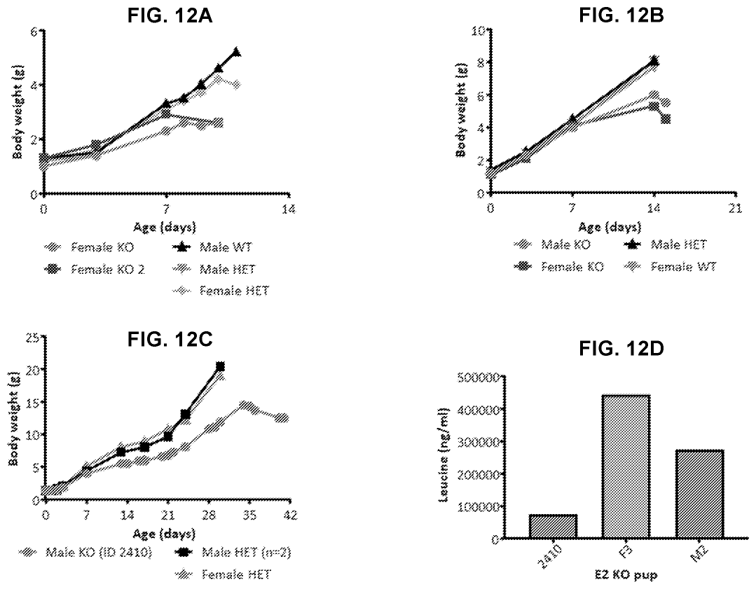

FIGS. 12A to 12D show an intravenous LNP encapsulated mRNA administration extends survival, increases body weight, and reduced serum leucine levels of classic MSUD mice. We administered litters of newborn E2 KO mice, and heterozygous (HET) and wild type (WT) littermates, with 2 mpk of LNP encapsulated mRNA for the three BCKDH subunits (E1a/E1b/E2) IV on days 0 and 3 of life via the facial vein. Starting on day 7, mice received weekly or biweekly IV administrations of LNPs via either the retro-orbital or tail veins. At day 21 and onward dosing was switched to biweekly. Mice were followed for survival and body weight (FIGS. 12A, 12B, and 12C) during the in-life phase of the study. Mice were euthanized based on clinical signs. Mouse 2410 was euthanized 24 hrs post-LNP injection, blood collected for serum leucine levels (FIG. 12D), and liver harvested for in situ hybridization (data not shown). While E2 KO mice have enhanced survival compared to untreated E2 KO mice, they still fail to thrive as indicated by body weight. While serum leucine levels are decreased in treated E2 KO mice, they have not been normalized to wild type levels.

Additionally, we examined effect of triple LNP (E1a/E1b/E2) injections in E1a MSUD KO litter. Studies were performed at doses ranging from 1-3 mpk. FIG. 13 shows a comparison of percent survival of cMSUD (E2 KO) and E1a MSUD KO mice with respect to rescuing of acute crisis in newborns following triple LNP injections. cMSUD mice administered with 2 mpk were able to survival past the neonatal lethality seen in this stain up to 40 days of life

### Example 5: Chronic Therapy Study with mRNA LNP in Adult iMSUD Mice

In this study, iMSUD mice were i.v. injected weekly with LNPs beginning at 21-28 days of age containing mRNA(s) for E2 only, E1a/E1b/E2 (triple), E2/GFP, or GFP only. Mice were also injected with vehicle (PBS) as a control. Survival and body weight were evaluated throughout the study, and mice were bled at 24 hours post LNP administration for BCAA analysis.

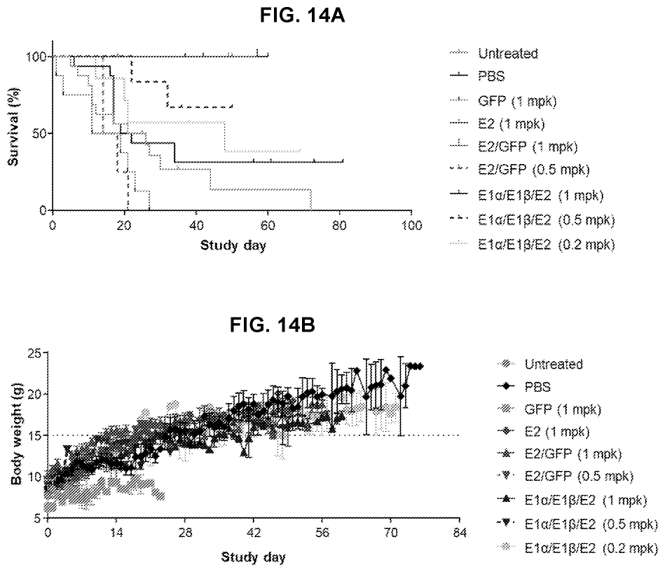

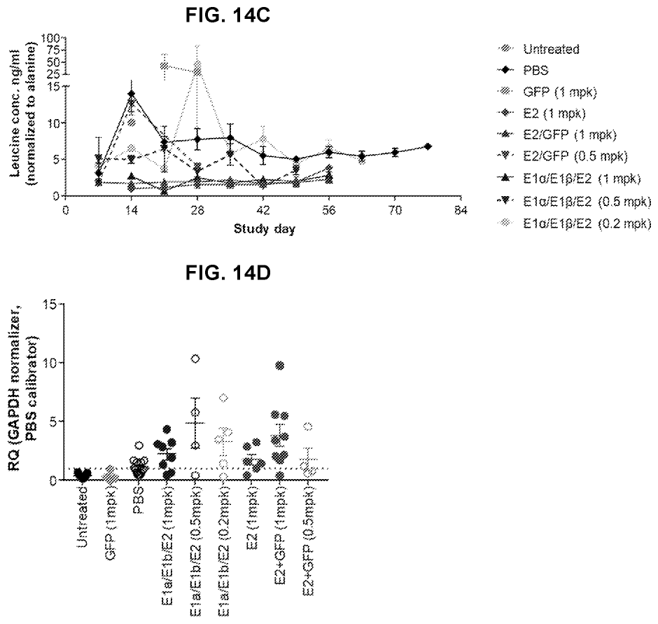

IV injections, either retroorbital (ROV) or tail vein (TV), were administered depending on the size of the iMSUD mouse, every 7 days from P21-28 onwards. iMSUD mice were dosed with: E2 only (1 mpk), E2.GFP (0.5 and 1 mpk), E1a/E1b/E2 (0.2, 0.5, and 1 mpk), GFP (1 mpk), PBS or untreated. Mice were evaluated for survival, body weight, serum BCAAs, and liver RNA levels by RT-PCR and ISH. FIG. 14A shows percent survival. Mice administered with 1 mpk of GFP LNP died within the first 30 days of the study. Dose of 1 mpk of triple LNP conferred an increase in survival. Decreasing the dose to 0.5 mpk of triple LNP showed reduced survival compared to the high dose group. FIG. 14B shows body weight changes. Dose of 1 mpk of triple LNP conferred an increase in body weights relative to mice administered with 1 mpk GFP LNP. All LNP treatment groups displayed increased body weight gain relative to GFP LNP treated controls. FIG. 14C shows normalized levels of leucine throughout the study. Mice that received treatment LNPs have reduced serum BCAA concentrations relative to both GFP LNP treated mice and levels historically observed in untreated iMSUD mice. However, only the high dose reduced BCAA levels to baseline levels observed in WT mice. FIG. 14D shows liver RNA levels of E2 evaluated by RT-PCR. Mice that received treatment LNPs have increase E2 RNA levels at necropsy (24 hrs final post-LNP dose). Liver tissue was additionally analyzed with in situ hybridization (ISH) with various BCKDH probes including a DBT probe that was designed to avoid endogenous hypomorph product (data not shown), and confirmed the expression levels of E1a, E1b, E2 as observed by RT-PCR.

### Example 6: Chronic Therapy Study with mRNA LNP in Newborn iMSUD Mice

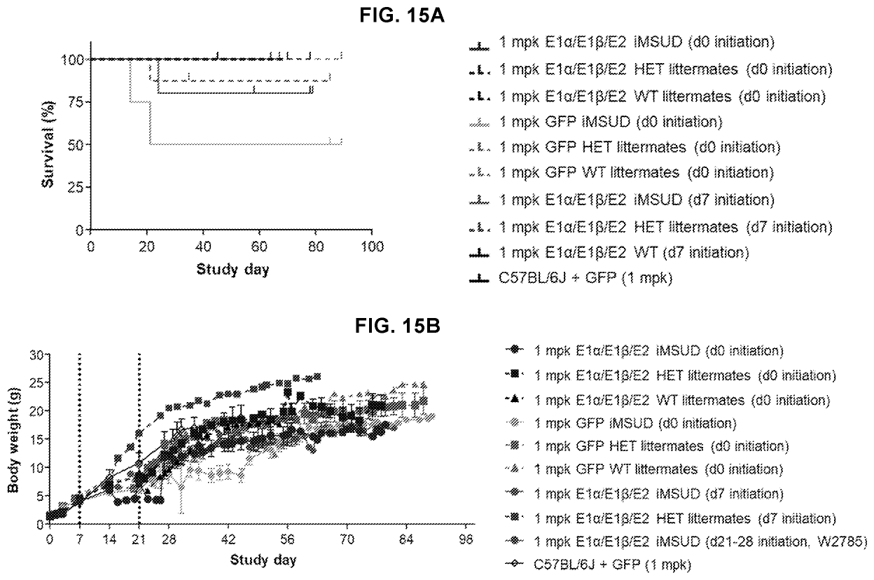

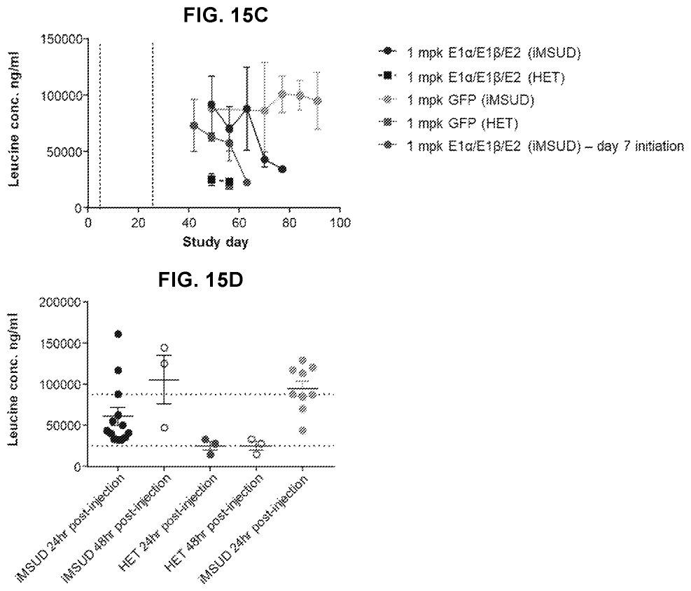

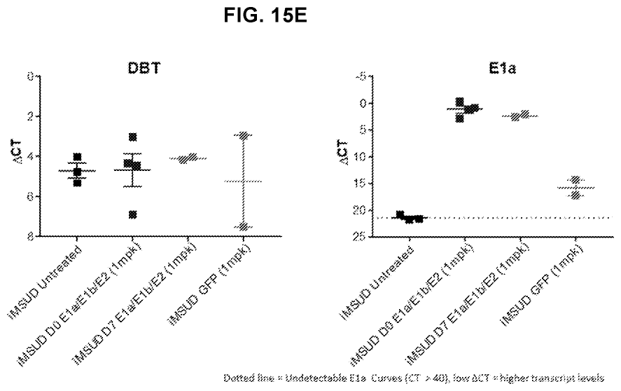

In this study, litters of iMSUD were administered with the newborn formulation of the triple LNP on days 0 and 3 by IV injection. Thereafter mice received weekly or twice a weekly IV injections of the adult formulation of the triple LNP or eGFP LNP (starting at day 7). Mice were monitored for survival (FIG. 15A) and body weight (FIG. 15B) throughout the in-life phase of the study. Mice were euthanized 24 hrs after final LNP injection. Mice were additionally evaluated for serum BCAAs (FIGS. 15C and 15D, and liver RNA levels by RT-QPCR (FIG. 17E) and ISH (data not shown). A decrease in serum leucine concentration over time was observed in iMSUD mice treated at 1 mpk E1α/E1β/E2 in comparison to GFP LNP treated group (FIG. 15C). Mice were euthanized at 24 hrs post final LNP treatment. A DBT probe used for RT-QPCR does not differentiate between endogenous human (pre-existing in iMSUD) and LNP delivered human mRNA (FIG. 15E). An E1a probe shows high expression (transcription) levels detectable post 24 hrs LNP treatment (FIG. 15E). ISH showed increased levels of RNA levels detected with E1a and E2 probes at day 7 with 1 mpk dose delivered.

### Example 7: A Pharmacokinetic Study of mRNA LNP Administered in Adult C57BL/6J Mice

In initial study, adult male C57BL/6 mice were administered with 1 mg/kg (mpk) of E1a/E1b/E2 or E2 LNP to determine the pharmacokinetic (PK) profiles of mRNA and protein in the liver at 24 hrs post-injection. Liver samples were analyzed by ISH for RNA levels. At 24-hours post LNP injection, E1a and E2 expression was observed (data not shown). The study was then repeated with E1a/E1b/E2, E2 LNP and E2/GFP LNP and additional time points were measured. Adult male C57BL/6 mice were administered with 1 mg/kg of E1a/E1b/E2 LNP or PBS to determine the PK profiles of mRNA and protein in the liver over time. LNPs were administered IV and mice were sacrificed at 6 hrs, 24 hrs, 48 hrs, 72 hrs, and 7 days post-injection. Liver samples were analyzed by qRT-PCR and ISH for RNA levels, and by Western blot and IHC for protein levels. The results from qRT-PCR indicated that E2 and E1b transcripts were detectable most strongly at 6 hrs post injection, whereas E1a transcripts were detectable strongly at both 6 hrs and 24 hrs, indicating possible greater stability in this mRNA. The mRNA levels were still detectable up to 72 hrs post injection of all transcripts, although to a greatly diminished level relative to 6 hrs post injection. ISH results mirrored the qRT-PCR results, with maximal mRNA visible at 6 hrs for E2 and E1b probes, and at both 6 hrs and 24 hrs for the E1a probe.

### REFERENCES

1. Hinton C F, Mai C T, Nabukera S K, Botto L D, Feuchtbaum L, Romitti P A, et al. Developing a public health-tracking system for follow-up of newborn screening metabolic conditions: a four-state pilot project structure and initial findings. Genet Med. 2014; 16(6):484-90.
2. Therrell B L, Jr., Lloyd-Puryear M A, Camp K M, Mann M Y. Inborn errors of metabolism identified via newborn screening: Ten-year incidence data and costs of nutritional interventions for research agenda planning. Mol Genet Metab. 2014; 113(1-2):14-26.
3. Nellis M M, Kasinski A, Carlson M, Allen R, Schaefer A M, Schwartz E M, et al. Relationship of causative genetic mutations in maple syrup urine disease with their clinical expression. Mol Genet Metab. 2003; 80(1-2):189-95.
4. Ali E Z, Ngu L H. Fourteen new mutations of BCKDHA, BCKDHB and DBT genes associated with maple syrup urine disease (MSUD) in Malaysian population. Mol Genet Metab Rep. 2018; 17:22-30.
5. Henneke M, Flaschker N, Helbling C, Muller M, Schadewaldt P, Gartner J, et al. Identification of twelve novel mutations in patients with classic and variant forms of maple syrup urine disease. Hum Mutat. 2003; 22(5):417.
6. Rodriguez-Pombo P, Navarrete R, Merinero B, Gomez-Puertas P, Ugarte M. Mutational spectrum of maple syrup urine disease in Spain. Hum Mutat. 2006; 27(7):715.
7. Morton D H, Strauss K A, Robinson D L, Puffenberger E G, Kelley R I. Diagnosis and treatment of maple syrup disease: a study of 36 patients. Pediatrics. 2002; 109(6):999-1008.
8. Strauss K A, Carson V J, Soltys K, Young M E, Bowser L E, Puffenberger E G, et al. Branched-chain alpha-ketoacid dehydrogenase deficiency (maple syrup urine disease): Treatment, biomarkers, and outcomes. Mol Genet Metab. 2020.
9. Mazariegos G V, Morton D H, Sindhi R, Soltys K, Nayyar N, Bond G, et al. Liver transplantation for classical maple syrup urine disease: long-term follow-up in 37 patients and comparative United Network for Organ Sharing experience. J Pediatr. 2012; 160(1):116-21 e1.
10. Suryawan A, Hawes J W, Harris R A, Shimomura Y, Jenkins A E, Hutson S M. A molecular model of human branched-chain amino acid metabolism. Am J Clin Nutr. 1998; 68(1):72-81.
11. Roda K M O, Vincenzi R, Fonseca E A, Benavides M, Turine P, Afonso R C, et al. Domino Liver Transplant in Maple Syrup Urine Disease: Technical Details of Cases in Which the First Surgery Involved a Living Donor. Transplantation. 2019; 103(3):536-43.
12. Badell I R, Hanish S I, Hughes C B, Hewitt W R, Chung R T, Spivey J R, et al. Domino liver transplantation in maple syrup urine disease: a case report and review of the literature.

Transplant Proc. 2013; 45(2):806-9.
13. Wang L, Calcedo R, Bell P, Lin J, Grant R L, Siegel D L, et al. Impact of pre-existing immunity on gene transfer to nonhuman primate liver with adeno-associated virus 8 vectors.

Hum Gene Ther. 2011; 22(11):1389-401.
14. Greig J A, Calcedo R, Grant R L, Peng H, Medina-Jaszek C A, Ahonkhai O, et al. Intramuscular administration of AAV overcomes pre-existing neutralizing antibodies in rhesus macaques. Vaccine. 2016; 34(50):6323-9.
15. Homanics G E, Skvorak K, Ferguson C, Watkins S, Paul H S. Production and characterization of murine models of classic and intermediate maple syrup urine disease. BMC Med Genet. 2006; 7:33.
16. Skvorak K J. Animal models of maple syrup urine disease. J Inherit Metab Dis. 2009; 32(2):229-46.
17. Gao G, Lu Y, Calcedo R, Grant R L, Bell P, Wang L, et al. Biology of AAV serotype vectors in liver-directed gene transfer to nonhuman primates. Mol Ther. 2006; 13(1):77-87.
18. Hinderer C, Katz N, Buza E L, Dyer C, Goode T, Bell P, et al. Severe Toxicity in Nonhuman Primates and Piglets Following High-Dose Intravenous Administration of an Adeno-Associated Virus Vector Expressing Human SMN. Hum Gene Ther. 2018.
19. Greig J A, Peng H, Ohlstein J, Medina-Jaszek C A, Ahonkhai O, Mentzinger A, et al. Intramuscular injection of AAV8 in mice and macaques is associated with substantial hepatic targeting and transgene expression. pLoS One. 2014; 9(11):e112268.
20. Bell P, Moscioni A D, McCarter R J, Wu D, Gao G, Hoang A, et al. Analysis of tumors arising in male B6C3F1 mice with and without AAV vector delivery to liver. Mol Ther. 2006; 14(1):34-44.
21. Wang L, Wang H, Bell P, McMenamin D, Wilson J M. Hepatic gene transfer in neonatal mice by adeno-associated virus serotype 8 vector. Hum Gene Ther. 2012; 23(5):533-9.

TABLE
(Sequence Listing Free Text)
The following information is provided for sequences
containing free text under numeric identifier <223>.

| SEQ ID NO: | |
|---|---|
| (containing | |
| free text) | Free text under <223> |
| 1 | <223> mature chain |
| 2 | <223> Engineered E2 coding sequence |
| 3 | <223> engineered nucleic sequence encoding human E1A |
| 4 | <223> Synthetic Construct |
| 5 | <223> engineered sequence encoding human E1B subunit |
| | protein |
| 6 | <223> Synthetic Construct |
| 7 | <223> CB7 Promoter Sequence |
| 8 | <223> CI Intron sequence |
| 9 | <223> SV40 Poly A |
| 10 | <223> TBG promoter |
| 11 | <223> WPRE |
| 12 | <223> bovine growth hormone (BGH) polyA |
| 13 | <223> alpha mic/bik |
| 14 | <223> SV40 misc intron |
| 15 | <223> '′ITR w Deletion 22 bp |
| 16 | <223> 2R5 S |
| 17 | <223> shortened MCK promoter |
| 18 | <223> TBG promoter with enhancer |
| 19 | <223> Promega intron sequence |
| 20 | <223> expression cassette |
| | <220> |
| | <221> repeat_region |
| | <222> (1) . . . (168) |
| | <223> 5′ITR |
| | <220> |
| | <221> misc_feature |
| | <222> (211) . . . (907) |
| | <223> TBG promoter w/enhancers |
| | <220> |
| | <221> enhancer |
| | <222> (211) . . . (310) |
| | <223> alpha mic/bik enhancer |
| | <220> |
| | <221> enhancer |
| | <222> (317) . . . (416) |
| | <223> alpha mic/bik enhancer |
| | <220> |
| | <221> Intron |
| | <222> (939) . . . (1071) |
| | <223> SV40 misc intron (Promega) |
| | <220> |
| | <221> CDS |
| | <222> (1089) . . . (2426) |
| | <223> hE1Aco |
| | <220> |
| | <221> misc_feature |
| | <222> (2449) . . . (2990) |
| | <223> WRPE |
| | <220> |
| | <221> polyA_signal |
| | <222> (2997) . . . (3211) |
| | <223> BGH pA |
| | <220> |
| | <221> misc_feature |
| | <222> (3253) . . . (3298) |
| | <223> Additional AAV sequences |
| | <220> |
| | <221> repeat_region |
| | <222> (3261) . . . (3428) |
| | <223> 3′ITR |
| 21 | <223> Synthetic Construct |
| 22 | <223> expression cassette |
| | <220> |
| | <221> repeat_region |
| | <222> (1) . . . (168) |
| | <223> 5'ITR |
| | <220> |
| | <221> enhancer |
| | <222> (211) . . . (310) |
| | <223> alpha mic/bik |
| | <220> |
| | <221> enhancer |
| | <222> (317) . . . (416) |
| | <223> alpha mic/bik |
| | <220> |
| | <221> misc_feature |
| | <222> (431) . . . (907) |
| | <223> TBG promoter |
| | <220> |
| | <221> Intron |
| | <222> (939) . . . (1071) |
| | <223> SV40 misc intron (Promega) |
| | <220> |
| | <221> CDS |
| | <222> (1092) . . . (2543) |
| | <223> hMSUD-E2co |
| | <220> |
| | <221> polyA_signal |
| | <222> (2550) . . . (2764) |
| | <223> BGHpolyA |
| | <220> |
| | <221> misc_feature |
| | <222> (2806) . . . (2851) |
| | <223> Additional AAV sequences |
| | <220> |
| | <221> repeat_region |
| | <222> (2814) . . . (2981) |
| | <223> 3′ITR |
| 23 | <223> Synthetic Construct |
| 24 | <223> expression cassette |
| | <220> |
| | <221> repeat_region |
| | <222> (1) . . . (168) |
| | <223> 5′ITR |
| | <220> |
| | <221> enhancer |
| | <222> (211) . . . (310) |
| | <223> alpha mic/bik |
| | <220> |
| | <221> enhancer |
| | <222> (317) . . . (416) |
| | <223> alpha mic/bik |
| | <220> |
| | <221> misc_feature |
| | <222> (431) . . . (907) |
| | <223> TBG promoter |
| | <220> |
| | <221> Intron |
| | <222> (939) . . . (1071) |
| | <223> SV40 misc intron (Promega) |
| | <220> |
| | <221> misc_feature |
| | <222> (1086) . . . (1091) |
| | <223> Kozak |
| | <220> |
| | <221> CDS |
| | <222> (1092) . . . (2543) |
| | <223> hMSUD-E2co |
| | <220> |
| | <221> misc_feature |
| | <222> (2559) . . . (3100) |
| | <223> WRPE |
| | <220> |
| | <221> polyA_signal |
| | <222> (3107) . . . (3321) |
| | <223> BGHpolyA |
| | <220> |
| | <221> misc_feature |
| | <222> (3363) . . . (3408) |
| | <223> additional AAV sequences |
| | <220> |
| | <221> repeat_region |
| | <222> (3371) . . . (3538) |
| | <223> 3′ITR |
| 25 | <223> Synthetic Construct |
| 26 | <223> expression cassette |
| | <220> |
| | <221> repeat_region |
| | <222> (1) . . . (108) |
| | <223> Repeat Region 1 5′ITR |
| | <220> |
| | <221> repeat_region |
| | <222> (182) . . . (563) |
| | <223> Repeat region 1 - CMV IE promoter |
| | <220> |
| | <221> promoter |
| | <222> (566) . . . (847) |
| | <223> Promoter E1 CB promoter |
| | <220> |
| | <221> TATA_signal |
| | <222> (820) . . . (823) |
| | <223> TATA signal 1 |
| | <220> |
| | <221> Intron |
| | <222> (940) . . . (1911) |
| | <223> chicken beta actin intron |
| | <220> |
| | <221> CDS |
| | <222> (1924) . . . (3372) |
| | <223> hMSUD-E2co |
| | <220> |
| | <221> polyA_signal |
| | <222> (3386) . . . (3617) |
| | <223> PolyA SV40 |
| | <220> |
| | <221> repeat_region |
| | <222> (3682) . . . (3811) |
| | <223> Repeat Region - 3′ITR |
| 27 | <223> Synthetic Construct |
| 28 | <223> expression cassette |
| | <220> |
| | <221> repeat_region |
| | <222> (1) . . . (108) |
| | <223> Repeat region - 5′ITR |
| | <220> |
| | <221> enhancer |
| | <222> (175) . . . (385) |
| | <223> 2R5 S |
| | <220> |
| | <221> enhancer |
| | <222> (386) . . . (596) |
| | <223> 2R5 S |
| | <220> |
| | <221> enhancer |
| | <222> (596) . . . (806) |
| | <223> 2R5 S |
| | <220> |
| | <221> promoter |
| | <222> (807) . . . (952) |
| | <223> shortened MCK promoter |
| | <220> |
| | <221> Intron |
| | <222> (979) . . . (1111) |
| | <223> PI (promega intron) |
| | <220> |
| | <221> CDS |
| | <222> (1140) . . . (2591) |
| | <223> hMSUD-E2co |
| | <220> |
| | <221> polyA_signal |
| | <222> (2602) . . . (2833) |
| | <223> poly A SV40 |
| | <220> |
| | <221> repeat_region |
| | <222> (2898) . . . (3027) |
| | <223> 3′ITR |
| 29 | <223> Synthetic Construct |
| 30 | <223> mRNA-E2 |
| 31 | <223> mRNA-E1a |
| 32 | <223> mRNA-E1b |

All publications cited in this specification are incorporated herein by reference in their entireties. U.S. Provisional Patent Application No. 62/864,263, filed Jun. 20, 2019, U.S. Provisional Patent Application No. 63/016,240, filed Apr. 27, 2020 and the SEQ ID NOs which are referenced herein and which appear in the appended Sequence Listing labeled “UPN-19-8936PCT_ST25.txt” are incorporated by reference. While the invention has been described with reference to particular embodiments, it will be appreciated that modifications can be made without departing from the spirit of the invention. Such modifications are intended to fall within the scope of the appended claims.

1. A vector comprising at least one nucleic acid sequence encoding one or more subunit proteins from a human branched-chain alpha-keto acid dehydrogenase (BCKDH) operably linked to a regulatory sequence which directs expression of the subunit protein in a target cell, wherein the at least one nucleic acid sequence is selected from:
(a) a nucleic acid sequence of SEQ ID NO: 2 or a sequence at least 95% identical to SEQ ID NO: 2 encoding a BCKDH E2 subunit protein;
(b) a nucleic acid sequence of SEQ ID NO: 3 or a sequence at least 95% identical to SEQ ID NO: 3 encoding a BCKDH E1A subunit protein;
(c) a nucleic acid sequence of SEQ ID NO: 5 or a sequence at least 95% identical to SEQ ID NO: 5 encoding a BCKDH E1B subunit protein.
(d) an E2 mRNA sequence of SEQ ID NO: 30 or a sequence at least 95% identical to SEQ ID NO: 30;
(e) a E1A mRNA sequence of SEQ ID NO: 31 or a sequence at least 95% identical to SEQ ID NO: 31; and/or
(f) a E1B mRNA sequence of SEQ ID NO: 32 or a sequence at least 95% identical to SEQ ID NO: 32.

2. The vector according to claim 1, wherein the vector comprises nucleic acid sequences encoding two or more of E2, E1A and E1B proteins.

3. A composition comprising at least one lipid nanoparticle comprising one or more of:
(a) an E2 mRNA sequence of SEQ ID NO: 30 or a sequence at least 95% identical thereto;
(b) an E1A mRNA sequence of SEQ ID NO: 31 or a sequence at least 95% identical thereto; and/or
(c) an E1B mRNA sequence of SEQ ID NO: 31 or a sequence at least 95% identical thereto.

4. The composition according to claim 2, wherein the composition comprises two or more of E2 mRNA, E1A mRNA, and/or E1B mRNA.

5. The composition according to claim 4, wherein the composition comprises a lipid nanoparticle comprising the mRNA.

6. A recombinant AAV (rAAV) comprising an AAV capsid and a vector genome packaged therein, wherein the vector genome comprises an AAV 5′ inverted terminal repeat (ITR), an engineered nucleic acid sequence encoding a E2 subunit protein from a human branched-chain alpha-keto acid dehydrogenase (BCKDH), a regulatory sequence which directs expression of E2 in a target cell, and an AAV 3′ ITR, wherein the E2 coding sequence is at least 95% identical to SEQ ID NO: 2.

7. The rAAV according to claim 1, wherein the E2 coding sequence is SEQ ID NO: 2.

8. A recombinant AAV (rAAV) comprising an AAV capsid and a vector genome packaged therein, wherein the vector genome comprises an AAV 5′ inverted terminal repeat (ITR), an engineered nucleic acid sequence encoding a regulatory sequence which directs expression of a E1A subunit protein from a human branched-chain alpha-keto acid dehydrogenase (BCKDH) in a target cell, and an AAV 3′ ITR, wherein the E1A coding sequence is at least 95% identical to SEQ ID NO: 3.

9. The rAAV according to claim 8, wherein the E1A coding sequence is SEQ ID NO: 3.

10. A recombinant AAV (rAAV) comprising an AAV capsid and a vector genome packaged therein, wherein the vector genome comprises an AAV 5′ inverted terminal repeat (ITR), an engineered nucleic acid sequence encoding a regulatory sequence which directs expression of a E1B subunit protein from a human branched-chain alpha-keto acid dehydrogenase (BCKDH) in a target cell, and an AAV 3′ ITR, wherein the E1B coding sequence is at least 95% identical to SEQ ID NO: 5.

11. The rAAV according to claim 10, wherein the E1B coding sequence is SEQ ID NO: 5.

12. The vector according to claim 1 or 2, the composition according to claim 3 or 4, or the rAAV according to any one of claims 5 to 11, wherein the vector genome comprises a muscle-specific promoter.

13. The vector according to claim 1 or 2, the composition according to claim 3 or 4, or the rAAV according to any one of claims 5 to 11, wherein the vector genome comprises a liver-specific promoter.

14. The rAAV according to any of claims 5 to 11, wherein the AAV capsid is an AAV9 capsid.

15. The rAAV according to any of claims 5 to 11, wherein the AAV capsid is an AAV1 capsid.

16. The rAAV according to any of claims 5 to 11, wherein the AAV capsid is an AAV8 capsid.

17. The vector according to claim 1 or 2, the composition according to claim 3 or 4, or the rAAV according to any one of claims 5 to 11, wherein the regulatory sequences comprise a CB7 promoter.

18. A non-viral vector comprising an mRNA, wherein mRNA sequence comprises a 5′ cap, a 5′ untranslated region (UTR), an engineered nucleic acid sequence encoding a E2 subunit protein from a human branched-chain alpha-keto acid dehydrogenase (BCKDH), a regulatory sequence which directs expression of E2 in a target cell, a 3′ UTR, and a poly(A) tail wherein the E2 coding sequence is at least 95% identical to SEQ ID NO: 30.

19. A non-viral vector comprising an mRNA, wherein mRNA sequence comprises a 5′ cap, a 5′ untranslated region (UTR), an engineered nucleic acid sequence encoding a E1A subunit protein from a human branched-chain alpha-keto acid dehydrogenase (BCKDH), a regulatory sequence which directs expression of E1A in a target cell, a 3′ UTR, and a poly(A) tail wherein the E1A coding sequence is at least 95% identical to SEQ ID NO: 31.

20. A non-viral vector comprising an mRNA, wherein mRNA sequence comprises a 5′ cap, a 5′ untranslated region (UTR), an engineered nucleic acid sequence encoding a E1B subunit protein from a human branched-chain alpha-keto acid dehydrogenase (BCKDH), a regulatory sequence which directs expression of E1B in a target cell, a 3′ UTR, and a poly(A) tail wherein the E1B coding sequence is at least 95% identical to SEQ ID NO: 32.

21. A pharmaceutical composition comprising the vector according to claim 1 or 2, the composition according to claim 3 or 4, the rAAV according to any one of claims 5 to 11, the non-viral vector according to any one of claims 18 to 20, or combinations thereof, in a suspension buffer.

22. The pharmaceutical composition according to claim 21, which is formulated for delivery via intramuscular or intravenous injection.

23. A vector according to claim 1 or 2, a composition according to claim 3 or 4, or a rAAV according to any one of claims 5 to 11, the non-viral vector according to any one of claims 18 to 20, or combinations thereof, for use in the treatment of Maple Syrup Urine Disease in a subject in need thereof.

24. The vector according to claim 1 or 2, a composition according to claim 3 or 4, or a rAAV according to any one of claims 5 to 11, the non-viral vector according to any one of claims 18 to 20, or combinations thereof, wherein the vector, composition or rAAV is formulated for combined targeting of the liver and muscle.

25. Use of a vector according to claim 1 or 2, a composition according to claim 3 or 4, or a rAAV according to any one of claims 5 to 11, the non-viral vector according to any one of claims 18 to 20, or combinations thereof, in the manufacture of a medicament for treatment of Maple Syrup Urine Disease in a subject in need thereof.

26. Use according to claim 22, wherein the medicament is adapted for co-administration to the liver and muscle.

27. A method for treating Maple Syrup Urine Disease comprising co-administering at least one vector stock comprising a nucleic acid sequence encoding one or more of a BCKDH E2A subunit protein, a BCKDH E1B subunit protein, and/or a BCKDH E1A subunit protein under control of regulatory sequences which direct expression in liver and muscle.

28. The method according to claim 24, wherein the at least one vector stock comprises a vector according to claim 1 or 2, a composition according to claim 3 or 4, or a rAAV according to any one of claims 5 to 11, the non-viral vector according to any one of claims 18 to 20, or combinations thereof.

29. A combination regimen for treating patients having Maple Syrup Urine Disease comprising co-delivering to muscle and liver at least one composition comprising at least one BCKDH nucleic acid encoding an E1A, E1B and/or E2 subunit protein under control of regulatory sequences which direct expression thereof in muscle and liver.

30. The combination regimen according to claim 29, wherein the at least one composition comprises one or more vector according to claim 1 or 2, one or more composition according to claim 3 or 4, one or more rAAV according to any one of claims 5 to 11, one or more non-viral vector according to any one of claims 18 to 20, or combinations thereof.


SEQUENCE LISTING (32 sequences)

SEQ ID NO: 1 - protein, 482 aa, Homo sapiens
  TRANSIT (1)..(61)
  MISC_FEATURE (62)..(482): mature chain
MAAVRMLRTWSRNAGKLICVRYFQTCGNVHVLKPNYVCFFGYPSFKYSHPHHFLKTTAALRGQVVQFKLSDIGEGIREVTVKEWYVKEGDTVSQFDSICEVQSDKASVTITSRYDGVIKKLYYNLDDIAYVGKPLVDIETEALKDSEEDVVETPAVSHDEHTHQEIKGRKTLATPAVRRLAMENNIKLSEVVGSGKDGRILKEDILNYLEKQTGAILPPSPKVEIMPPPPKPKDMTVPILVSKPPVFTGKDKTEPIKGFQKAMVKTMSAALKIPHFGYCDEIDLTELVKLREELKPIAFARGIKLSFMPFFLKAASLGLLQFPILNASVDENCQNITYKASHNIGIAMDTEQGLIVPNVKNVQICSIFDIATELNRLQKLGSVGQLSTTDLTGGTFTLSNIGSIGGTFAKPVIMPPEVAIGALGSIKAIPRFNQKGEVYKAQIMNVSWSADHRVIDGATMSRFSNLWKSYLENPAFMLLDLK

SEQ ID NO: 2 - DNA, 1449 bp, Artificial Sequence
  Engineered E2 coding sequence
atggccgccgtcagaatgctgagaacctggtccagaaacgccggcaagctgatctgcgtgcggtactttcagacctgcggcaatgtgcacgtgctgaagcccaactacgtgtgcttcttcggctaccccagcttcaagtactctcaccctcaccactttctcaagaccaccgccgctctgagaggacaggtggtgcagttcaagctgagcgacatcggcgagggcatcagagaagtgaccgtgaaagagtggtacgtcaaagagggcgacaccgtcagccagttcgacagcatctgtgaagtgcagagcgacaaggccagcgtgaccatcaccagcagatacgacggcgtgatcaagaagctgtactacaacctggacgatatcgcctacgtgggcaagcccctggtggacattgaaacagaggccctgaaggacagcgaagaggacgtggtggaaacccctgccgtgtctcacgacgagcacacccaccaagagatcaagggcagaaagaccctggccacaccagctgttagacggctggccatggaaaacaacatcaagctgtccgaggtcgtcggcagcggcaaggatggcagaatcctgaaagaggacatcctgaactacctcgagaagcagaccggcgccatcctgcctccatctccaaaggtggaaatcatgcctcctccacctaagcctaaggacatgaccgtgcctatcctggtgtccaagcctcctgtgttcaccggcaaggacaagaccgagccaatcaagggcttccagaaagccatggtcaagaccatgagcgctgccctgaagatccctcactttggctactgcgacgagatcgacctgaccgagctggttaagctgagagaggaactgaagcctatcgccttcgccagaggcatcaaactgagcttcatgccgttctttctgaaggccgcctctctgggcctgctgcagttccctattctgaacgccagcgtggacgagaactgccagaacatcacctacaaggcctctcacaacatcggaatcgccatggacacagagcagggcctgatcgtgcccaacgtgaagaacgtgcagatttgctccatcttcgacattgccaccgagctgaaccggctgcagaaactgggatctgtgggccagctgagcaccaccgatcttacaggcggcaccttcacactgtccaacatcggctctatcggcggcacattcgccaagcctgtgatcatgccaccagaggtggccattggagccctgggcagcattaaggccattcctcggttcaaccagaaaggcgaggtgtacaaggcccagatcatgaacgtgtcttggagcgccgaccacagagtgatcgatggcgccaccatgagcagattcagcaacctgtggaagtcctacctggaaaaccccgccttcatgctgctggacctgaagtga

SEQ ID NO: 3 - DNA, 1339 bp, Artificial Sequence
  engineered nucleic sequence encoding human E1A
  CDS (1)..(1338)
atggctgtggctattgctgctgccagagtgtggcggctgaatagaggactgtctcaggctgctctgctgctgctgagacaacctggtgcaagaggactggccagaagccatcctcctagacagcagcaacagttcagcagcctggacgacaagcctcagtttcctggcgcctccgccgagttcatcgacaagctggaattcatccagcctaacgtgatcagcggcatccccatctacagagtgatggacagacagggccagatcatcaaccccagcgaggaccctcacctgcccaaagaaaaggtgctgaagctgtacaagagcatgaccctgctgaacacaatggaccggatcctgtacgagagccagagacagggcagaatcagcttctacatgaccaactacggcgaagagggcacacacgtgggatctgctgccgctctggacaacaccgacctggtgtttggccagtacagagaagccggcgtgctgatgtaccgggactaccctctggaactgttcatggcccagtgctacggcaacatcagcgacctcggaaagggcagacagatgcctgtgcactacggctgcaaagaacggcacttcgtgaccatcagcagccctctggccacacagattcctcaggctgtgggagccgcctacgccgccaaaagagccaacgccaacagagtggtcatctgctactttggcgagggcgctgcttctgaaggcgacgctcacgccggcttcaatttcgccgccactctggagtgccccatcatctttttctgccggaacaacggctacgccatcagcacacctaccagcgagcagtatagaggcgacggaattgccgccagaggacctggctatggcatcatgagcatcagagtggacggcaacgacgtgttcgccgtgtacaacgccaccaaagaagccaggcggagagccgtggctgagaaccagccttttctgatcgaggccatgacctaccggatcggccaccacagcacctccgacgatagcagcgcctacagatccgtggacgaagtgaactactgggacaagcaggatcaccccatctccagactgcggcactacctgctgtctcaaggctggtgggacgaagaacaagagaaggcctggcggaagcagagccggcggaaagtgatggaagcctttgagcaggccgagcggaagcccaagcctaatcctaacctgctgttcagcgacgtgtaccaagagatgcctgctcagctgcgcaagcagcaagaaagcctggccagacacctgcagacctacggcgagcactacccactggaccacttcgacaaatgat

SEQ ID NO: 4 - protein, 445 aa, Artificial Sequence
  Synthetic Construct
MAVAIAAARVWRLNRGLSQAALLLLRQPGARGLARSHPPRQQQQFSSLDDKPQFPGASAEFIDKLEFIQPNVISGIPIYRVMDRQGQIINPSEDPHLPKEKVLKLYKSMTLLNTMDRILYESQRQGRISFYMTNYGEEGTHVGSAAALDNTDLVFGQYREAGVLMYRDYPLELFMAQCYGNISDLGKGRQMPVHYGCKERHFVTISSPLATQIPQAVGAAYAAKRANANRVVICYFGEGAASEGDAHAGFNFAATLECPIIFFCRNNGYAISTPTSEQYRGDGIAARGPGYGIMSIRVDGNDVFAVYNATKEARRRAVAENQPFLIEAMTYRIGHHSTSDDSSAYRSVDEVNYWDKQDHPISRLRHYLLSQGWWDEEQEKAWRKQSRRKVMEAFEQAERKPKPNPNLLFSDVYQEMPAQLRKQQESLARHLQTYGEHYPLDHFDK

SEQ ID NO: 5 - DNA, 1182 bp, Artificial Sequence
  engineered sequence encoding human E1B subunit protein
  CDS (1)..(1182)
atggctgttgttgctgctgcagctggatggctgctgagacttagagctgctggcgccgaaggccattggagaaggttgcctggtgctggactggccagaggctttctgcatcctgccgccacagtggaagacgccgctcagagaaggcaggtcgcccacttcaccttccagcctgatcctgagcctagagagtacggccagacacagaagatgaacctgttccagagcgtgacaagcgccctggacaacagcctggccaaagatcctaccgccgtgatcttcggagaggacgtggcattcggcggcgtgttcagatgtacagtgggcctgagagataagtacggcaaggaccgggtgttcaacacacccctgtgtgaacagggcatcgtcggcttcggaatcggaattgctgtgacaggcgccaccgccattgccgagattcagttcgccgactacatcttccccgccttcgaccagatcgtgaacgaggccgccaagtacagatacagaagcggcgacctgttcaactgcggcagcctgaccattagaagcccttggggctgtgttggacacggcgccctgtatcactctcagagccctgaggccttcttcgcccactgtcctggcatcaaggtggtcatccctagaagcccctttcaggccaagggcctgctgctgtcctgcatcgaggacaagaacccctgcatcttcttcgagcccaagatcctgtaccgggctgccgctgaggaagtgcctatcgagccctacaacatccctctgagccaggccgaagtgatccaagagggctccgatgtgacactggtggcctggggaacacaggtgcacgtgatcagagaagtggccagcatggctaaagagaagctgggcgtcagctgtgaagtgatcgacctgagaacaatcatcccctgggacgtcgacaccatctgcaagtccgtgatcaagaccggcagactgctgatctctcacgaggctcctctgacaggcggctttgcctctgagatcagctccaccgtgcaagaggagtgcttcctgaacctggaagccccaatcagcagagtgtgcggctacgacaccccttttccacacatctttgagcccttctacatccccgacaagtggaagtgctacgacgccctgcggaagatgatcaactactgataa

SEQ ID NO: 6 - protein, 392 aa, Artificial Sequence
  Synthetic Construct
MAVVAAAAGWLLRLRAAGAEGHWRRLPGAGLARGFLHPAATVEDAAQRRQVAHFTFQPDPEPREYGQTQKMNLFQSVTSALDNSLAKDPTAVIFGEDVAFGGVFRCTVGLRDKYGKDRVFNTPLCEQGIVGFGIGIAVTGATAIAEIQFADYIFPAFDQIVNEAAKYRYRSGDLFNCGSLTIRSPWGCVGHGALYHSQSPEAFFAHCPGIKVVIPRSPFQAKGLLLSCIEDKNPCIFFEPKILYRAAAEEVPIEPYNIPLSQAEVIQEGSDVTLVAWGTQVHVIREVASMAKEKLGVSCEVIDLRTIIPWDVDTICKSVIKTGRLLISHEAPLTGGFASEISSTVQEECFLNLEAPISRVCGYDTPFPHIFEPFYIPDKWKCYDALRKMINY

SEQ ID NO: 7 - DNA, 282 bp, Artificial Sequence
  CB7 Promoter Sequence
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 8 - DNA, 282 bp, Artificial Sequence; identical to SEQ ID NO: 7
  CI Intron sequence

SEQ ID NO: 9 - DNA, 232 bp, Artificial Sequence
  SV40 PolyA
cgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 10 - DNA, 477 bp, Artificial Sequence
  TBG promoter
agggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 11 - DNA, 542 bp, Artificial Sequence
  WPRE
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 12 - DNA, 215 bp, Artificial Sequence
  bovine growth hormone (BGH) polyA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 13 - DNA, 100 bp, Artificial sequence
  alpha mic/bik
aggttaatttttaaaaagcagtcaaaagtccaagtggcccttggcagcatttactctctctgtttgctctggttaataatctcaggagcacaaacattcc

SEQ ID NO: 14 - DNA, 133 bp, Artificial Sequence
  SV40 misc intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 15 - DNA, 108 bp, Artificial sequence
  5′ ITR w Deletion 22 bp
ctgcgcgctcgctcgctcactgaggccgcccgggcaaagcccgggcggcctcagtgagcgagcgagcgcgcagagagggagtggccaactccatcactaggggttcct

SEQ ID NO: 16 - DNA, 108 bp, Artificial Sequence; identical to SEQ ID NO: 15
  2R5 S

SEQ ID NO: 17 - DNA, 146 bp, Artificial sequence
  shortened MCK promoter
cctccctggggacagcccctcctggctagtcacaccctgtaggctcctctatataacccaggggcacaggggctgcccccgggtcacggggatcctctagacctccgaacggtaagagcctagcatgtagaactggttaccctgca

SEQ ID NO: 18 - DNA, 697 bp, Artificial Sequence
  TBG promoter with enhancer
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 19 - DNA, 133 bp, Artificial Sequence; identical to SEQ ID NO: 14
  Promega intron sequence

SEQ ID NO: 20 - DNA, 3428 bp, Artificial Sequence
  expression cassette
  repeat_region (1)..(168): 5′ITR
  misc_feature (211)..(907): TBG promoter w/enhancers
  enhancer (211)..(310): alpha mic/bik enhancer
  enhancer (317)..(416): alpha mic/bik enhancer
  Intron (939)..(1071): SV40 misc intron (Promega)
  CDS (1089)..(2426): hE1Aco
  misc_feature (2449)..(2990): WRPE
  polyA_signal (2997)..(3211): BGH pA
  misc_feature (3253)..(3298): Additional AAV sequences
  repeat_region (3261)..(3428): 3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggctgtggctattgctgctgccagagtgtggcggctgaatagaggactgtctcaggctgctctgctgctgctgagacaacctggtgcaagaggactggccagaagccatcctcctagacagcagcaacagttcagcagcctggacgacaagcctcagtttcctggcgcctccgccgagttcatcgacaagctggaattcatccagcctaacgtgatcagcggcatccccatctacagagtgatggacagacagggccagatcatcaaccccagcgaggaccctcacctgcccaaagaaaaggtgctgaagctgtacaagagcatgaccctgctgaacacaatggaccggatcctgtacgagagccagagacagggcagaatcagcttctacatgaccaactacggcgaagagggcacacacgtgggatctgctgccgctctggacaacaccgacctggtgtttggccagtacagagaagccggcgtgctgatgtaccgggactaccctctggaactgttcatggcccagtgctacggcaacatcagcgacctcggaaagggcagacagatgcctgtgcactacggctgcaaagaacggcacttcgtgaccatcagcagccctctggccacacagattcctcaggctgtgggagccgcctacgccgccaaaagagccaacgccaacagagtggtcatctgctactttggcgagggcgctgcttctgaaggcgacgctcacgccggcttcaatttcgccgccactctggagtgccccatcatctttttctgccggaacaacggctacgccatcagcacacctaccagcgagcagtatagaggcgacggaattgccgccagaggacctggctatggcatcatgagcatcagagtggacggcaacgacgtgttcgccgtgtacaacgccaccaaagaagccaggcggagagccgtggctgagaaccagccttttctgatcgaggccatgacctaccggatcggccaccacagcacctccgacgatagcagcgcctacagatccgtggacgaagtgaactactgggacaagcaggatcaccccatctccagactgcggcactacctgctgtctcaaggctggtgggacgaagaacaagagaaggcctggcggaagcagagccggcggaaagtgatggaagcctttgagcaggccgagcggaagcccaagcctaatcctaacctgctgttcagcgacgtgtaccaagagatgcctgctcagctgcgcaagcagcaagaaagcctggccagacacctgcagacctacggcgagcactacccactggaccacttcgacaaatgataaagatctgcccggccgatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - protein, 445 aa, Artificial Sequence; identical to SEQ ID NO: 4
  Synthetic Construct

SEQ ID NO: 22 - DNA, 2981 bp, Artificial Sequence
  expression cassette
  repeat_region (1)..(168): 5′ITR
  enhancer (211)..(310): alpha mic/bik
  enhancer (317)..(416): alpha mic/bik
  misc_feature (431)..(907): TBG promoter
  Intron (939)..(1071): SV40 misc intron (Promega)
  CDS (1092)..(2543): hMSUD-E2co
  polyA_signal (2550)..(2764): BGH polyA
  misc_feature (2806)..(2851): Additional AAV sequences
  repeat_region (2814)..(2981): 3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatggccgccgtcagaatgctgagaacctggtccagaaacgccggcaagctgatctgcgtgcggtactttcagacctgcggcaatgtgcacgtgctgaagcccaactacgtgtgcttcttcggctaccccagcttcaagtactctcaccctcaccactttctcaagaccaccgccgctctgagaggacaggtggtgcagttcaagctgagcgacatcggcgagggcatcagagaagtgaccgtgaaagagtggtacgtcaaagagggcgacaccgtcagccagttcgacagcatctgtgaagtgcagagcgacaaggccagcgtgaccatcaccagcagatacgacggcgtgatcaagaagctgtactacaacctggacgatatcgcctacgtgggcaagcccctggtggacattgaaacagaggccctgaaggacagcgaagaggacgtggtggaaacccctgccgtgtctcacgacgagcacacccaccaagagatcaagggcagaaagaccctggccacaccagctgttagacggctggccatggaaaacaacatcaagctgtccgaggtcgtcggcagcggcaaggatggcagaatcctgaaagaggacatcctgaactacctcgagaagcagaccggcgccatcctgcctccatctccaaaggtggaaatcatgcctcctccacctaagcctaaggacatgaccgtgcctatcctggtgtccaagcctcctgtgttcaccggcaaggacaagaccgagccaatcaagggcttccagaaagccatggtcaagaccatgagcgctgccctgaagatccctcactttggctactgcgacgagatcgacctgaccgagctggttaagctgagagaggaactgaagcctatcgccttcgccagaggcatcaaactgagcttcatgccgttctttctgaaggccgcctctctgggcctgctgcagttccctattctgaacgccagcgtggacgagaactgccagaacatcacctacaaggcctctcacaacatcggaatcgccatggacacagagcagggcctgatcgtgcccaacgtgaagaacgtgcagatttgctccatcttcgacattgccaccgagctgaaccggctgcagaaactgggatctgtgggccagctgagcaccaccgatcttacaggcggcaccttcacactgtccaacatcggctctatcggcggcacattcgccaagcctgtgatcatgccaccagaggtggccattggagccctgggcagcattaaggccattcctcggttcaaccagaaaggcgaggtgtacaaggcccagatcatgaacgtgtcttggagcgccgaccacagagtgatcgatggcgccaccatgagcagattcagcaacctgtggaagtcctacctggaaaaccccgccttcatgctgctggacctgaagtgataaagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - protein, 482 aa, Artificial Sequence; identical to SEQ ID NO: 1
  Synthetic Construct

SEQ ID NO: 24 - DNA, 3538 bp, Artificial Sequence
  expression cassette
  repeat_region (1)..(168): 5′ITR
  enhancer (211)..(310): alpha mic/bik
  enhancer (317)..(416): alpha mic/bik
  misc_feature (431)..(907): TBG promoter
  Intron (939)..(1071): SV40 misc intron (Promega)
  misc_feature (1086)..(1091): Kozak
  CDS (1092)..(2543): hMSUD-E2co
  misc_feature (2559)..(3100): WRPE
  polyA_signal (3107)..(3321): BGH polyA
  misc_feature (3363)..(3408): additional AAV sequences
  repeat_region (3371)..(3538): 3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatggccgccgtcagaatgctgagaacctggtccagaaacgccggcaagctgatctgcgtgcggtactttcagacctgcggcaatgtgcacgtgctgaagcccaactacgtgtgcttcttcggctaccccagcttcaagtactctcaccctcaccactttctcaagaccaccgccgctctgagaggacaggtggtgcagttcaagctgagcgacatcggcgagggcatcagagaagtgaccgtgaaagagtggtacgtcaaagagggcgacaccgtcagccagttcgacagcatctgtgaagtgcagagcgacaaggccagcgtgaccatcaccagcagatacgacggcgtgatcaagaagctgtactacaacctggacgatatcgcctacgtgggcaagcccctggtggacattgaaacagaggccctgaaggacagcgaagaggacgtggtggaaacccctgccgtgtctcacgacgagcacacccaccaagagatcaagggcagaaagaccctggccacaccagctgttagacggctggccatggaaaacaacatcaagctgtccgaggtcgtcggcagcggcaaggatggcagaatcctgaaagaggacatcctgaactacctcgagaagcagaccggcgccatcctgcctccatctccaaaggtggaaatcatgcctcctccacctaagcctaaggacatgaccgtgcctatcctggtgtccaagcctcctgtgttcaccggcaaggacaagaccgagccaatcaagggcttccagaaagccatggtcaagaccatgagcgctgccctgaagatccctcactttggctactgcgacgagatcgacctgaccgagctggttaagctgagagaggaactgaagcctatcgccttcgccagaggcatcaaactgagcttcatgccgttctttctgaaggccgcctctctgggcctgctgcagttccctattctgaacgccagcgtggacgagaactgccagaacatcacctacaaggcctctcacaacatcggaatcgccatggacacagagcagggcctgatcgtgcccaacgtgaagaacgtgcagatttgctccatcttcgacattgccaccgagctgaaccggctgcagaaactgggatctgtgggccagctgagcaccaccgatcttacaggcggcaccttcacactgtccaacatcggctctatcggcggcacattcgccaagcctgtgatcatgccaccagaggtggccattggagccctgggcagcattaaggccattcctcggttcaaccagaaaggcgaggtgtacaaggcccagatcatgaacgtgtcttggagcgccgaccacagagtgatcgatggcgccaccatgagcagattcagcaacctgtggaagtcctacctggaaaaccccgccttcatgctgctggacctgaagtgataaagatctgccggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - protein, 482 aa, Artificial Sequence; identical to SEQ ID NO: 1
  Synthetic Construct

SEQ ID NO: 26 - DNA, 3811 bp, Artificial Sequence
  expression cassette
  repeat_region (1)..(108): Repeat Region 1 5′ITR
  repeat_region (182)..(563): Repeat region 1 - CMV IE promoter
  promoter (566)..(847): Promoter E1 CB promoter
  TATA_signal (820)..(823): TATA signal 1
  Intron (940)..(1911): chicken beta actin intron
  CDS (1924)..(3372): hMSUD-E2co
  polyA_signal (3386)..(3617): PolyA  SV40
  repeat_region (3682)..(3811): Repeat Region - 3′ITR
ctgcgcgctcgctcgctcactgaggccgcccgggcaaagcccgggcggcctcagtgagcgagcgagcgcgcagagagggagtggccaactccatcactaggggttccttgtagttaatgattaacccgccatgctacttatctacgtagccatgctctaggaagatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggccgccgtcagaatgctgagaacctggtccagaaacgccggcaagctgatctgcgtgcggtactttcagacctgcggcaatgtgcacgtgctgaagcccaactacgtgtgcttcttcggctaccccagcttcaagtactctcaccctcaccactttctcaagaccaccgccgctctgagaggacaggtggtgcagttcaagctgagcgacatcggcgagggcatcagagaagtgaccgtgaaagagtggtacgtcaaagagggcgacaccgtcagccagttcgacagcatctgtgaagtgcagagcgacaaggccagcgtgaccatcaccagcagatacgacggcgtgatcaagaagctgtactacaacctggacgatatcgcctacgtgggcaagcccctggtggacattgaaacagaggccctgaaggacagcgaagaggacgtggtggaaacccctgccgtgtctcacgacgagcacacccaccaagagatcaagggcagaaagaccctggccacaccagctgttagacggctggccatggaaaacaacatcaagctgtccgaggtcgtcggcagcggcaaggatggcagaatcctgaaagaggacatcctgaactacctcgagaagcagaccggcgccatcctgcctccatctccaaaggtggaaatcatgcctcctccacctaagcctaaggacatgaccgtgcctatcctggtgtccaagcctcctgtgttcaccggcaaggacaagaccgagccaatcaagggcttccagaaagccatggtcaagaccatgagcgctgccctgaagatccctcactttggctactgcgacgagatcgacctgaccgagctggttaagctgagagaggaactgaagcctatcgccttcgccagaggcatcaaactgagcttcatgccgttctttctgaaggccgcctctctgggcctgctgcagttccctattctgaacgccagcgtggacgagaactgccagaacatcacctacaaggcctctcacaacatcggaatcgccatggacacagagcagggcctgatcgtgcccaacgtgaagaacgtgcagatttgctccatcttcgacattgccaccgagctgaaccggctgcagaaactgggatctgtgggccagctgagcaccaccgatcttacaggcggcaccttcacactgtccaacatcggctctatcggcggcacattcgccaagcctgtgatcatgccaccagaggtggccattggagccctgggcagcattaaggccattcctcggttcaaccagaaaggcgaggtgtacaaggcccagatcatgaacgtgtcttggagcgccgaccacagagtgatcgatggcgccaccatgagcagattcagcaacctgtggaagtcctacctggaaaaccccgccttcatgctgctggacctgaagtgataagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 27 - protein, 482 aa, Artificial Sequence; identical to SEQ ID NO: 1
  Synthetic Construct

SEQ ID NO: 28 - DNA, 3027 bp, Artificial Sequence
  expression cassette
  repeat_region (1)..(108): Repeat region - 5′ITR
  enhancer (175)..(385): 2R5 S
  enhancer (386)..(596): 2R5 S
  enhancer (596)..(806): 2R5 S
  promoter (807)..(952): shortened MCK promoter
  Intron (979)..(1111): PI (promega intron)
  CDS (1140)..(2591): hMSUD-E2co
  polyA_signal (2602)..(2833): polyA SV40
  repeat_region (2898)..(3027): 3′ITR
ctgcgcgctcgctcgctcactgaggccgcccgggcaaagcccgggcggcctcagtgagcgagcgagcgcgcagagagggagtggccaactccatcactaggggttccttgtagttaatgattaacccgccatgctacttatctacgtagccatgctctaggaagatcctctagccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccctccctggggacagcccctcctggctagtcacaccctgtaggctcctctatataacccaggggcacaggggctgcccccgggtcacggggatcctctagacctccgaacggtaagagcctagcatgtagaactggttaccctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgaattcgccaccatggccgccgtcagaatgctgagaacctggtccagaaacgccggcaagctgatctgcgtgcggtactttcagacctgcggcaatgtgcacgtgctgaagcccaactacgtgtgcttcttcggctaccccagcttcaagtactctcaccctcaccactttctcaagaccaccgccgctctgagaggacaggtggtgcagttcaagctgagcgacatcggcgagggcatcagagaagtgaccgtgaaagagtggtacgtcaaagagggcgacaccgtcagccagttcgacagcatctgtgaagtgcagagcgacaaggccagcgtgaccatcaccagcagatacgacggcgtgatcaagaagctgtactacaacctggacgatatcgcctacgtgggcaagcccctggtggacattgaaacagaggccctgaaggacagcgaagaggacgtggtggaaacccctgccgtgtctcacgacgagcacacccaccaagagatcaagggcagaaagaccctggccacaccagctgttagacggctggccatggaaaacaacatcaagctgtccgaggtcgtcggcagcggcaaggatggcagaatcctgaaagaggacatcctgaactacctcgagaagcagaccggcgccatcctgcctccatctccaaaggtggaaatcatgcctcctccacctaagcctaaggacatgaccgtgcctatcctggtgtccaagcctcctgtgttcaccggcaaggacaagaccgagccaatcaagggcttccagaaagccatggtcaagaccatgagcgctgccctgaagatccctcactttggctactgcgacgagatcgacctgaccgagctggttaagctgagagaggaactgaagcctatcgccttcgccagaggcatcaaactgagcttcatgccgttctttctgaaggccgcctctctgggcctgctgcagttccctattctgaacgccagcgtggacgagaactgccagaacatcacctacaaggcctctcacaacatcggaatcgccatggacacagagcagggcctgatcgtgcccaacgtgaagaacgtgcagatttgctccatcttcgacattgccaccgagctgaaccggctgcagaaactgggatctgtgggccagctgagcaccaccgatcttacaggcggcaccttcacactgtccaacatcggctctatcggcggcacattcgccaagcctgtgatcatgccaccagaggtggccattggagccctgggcagcattaaggccattcctcggttcaaccagaaaggcgaggtgtacaaggcccagatcatgaacgtgtcttggagcgccgaccacagagtgatcgatggcgccaccatgagcagattcagcaacctgtggaagtcctacctggaaaaccccgccttcatgctgctggacctgaagtgataagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 29 - protein, 482 aa, Artificial Sequence; identical to SEQ ID NO: 1
  Synthetic Construct

SEQ ID NO: 30 - RNA, 1449 nt, Artificial Sequence
  mRNA-E2
auggccgccgucagaaugcugagaaccugguccagaaacgccggcaagcugaucugcgugcgguacuuucagaccugcggcaaugugcacgugcugaagcccaacuacgugugcuucuucggcuaccccagcuucaaguacucucacccucaccacuuucucaagaccaccgccgcucugagaggacagguggugcaguucaagcugagcgacaucggcgagggcaucagagaagugaccgugaaagagugguacgucaaagagggcgacaccgucagccaguucgacagcaucugugaagugcagagcgacaaggccagcgugaccaucaccagcagauacgacggcgugaucaagaagcuguacuacaaccuggacgauaucgccuacgugggcaagccccugguggacauugaaacagaggcccugaaggacagcgaagaggacgugguggaaaccccugccgugucucacgacgagcacacccaccaagagaucaagggcagaaagacccuggccacaccagcuguuagacggcuggccauggaaaacaacaucaagcuguccgaggucgucggcagcggcaaggauggcagaauccugaaagaggacauccugaacuaccucgagaagcagaccggcgccauccugccuccaucuccaaagguggaaaucaugccuccuccaccuaagccuaaggacaugaccgugccuauccugguguccaagccuccuguguucaccggcaaggacaagaccgagccaaucaagggcuuccagaaagccauggucaagaccaugagcgcugcccugaagaucccucacuuuggcuacugcgacgagaucgaccugaccgagcugguuaagcugagagaggaacugaagccuaucgccuucgccagaggcaucaaacugagcuucaugccguucuuucugaaggccgccucucugggccugcugcaguucccuauucugaacgccagcguggacgagaacugccagaacaucaccuacaaggccucucacaacaucggaaucgccauggacacagagcagggccugaucgugcccaacgugaagaacgugcagauuugcuccaucuucgacauugccaccgagcugaaccggcugcagaaacugggaucugugggccagcugagcaccaccgaucuuacaggcggcaccuucacacuguccaacaucggcucuaucggcggcacauucgccaagccugugaucaugccaccagagguggccauuggagcccugggcagcauuaaggccauuccucgguucaaccagaaaggcgagguguacaaggcccagaucaugaacgugucuuggagcgccgaccacagagugaucgauggcgccaccaugagcagauucagcaaccuguggaaguccuaccuggaaaaccccgccuucaugcugcuggaccugaaguga

SEQ ID NO: 31 - RNA, 1339 nt, Artificial Sequence
  mRNA-E1a
auggcuguggcuauugcugcugccagaguguggcggcugaauagaggacugucucaggcugcucugcugcugcugagacaaccuggugcaagaggacuggccagaagccauccuccuagacagcagcaacaguucagcagccuggacgacaagccucaguuuccuggcgccuccgccgaguucaucgacaagcuggaauucauccagccuaacgugaucagcggcauccccaucuacagagugauggacagacagggccagaucaucaaccccagcgaggacccucaccugcccaaagaaaaggugcugaagcuguacaagagcaugacccugcugaacacaauggaccggauccuguacgagagccagagacagggcagaaucagcuucuacaugaccaacuacggcgaagagggcacacacgugggaucugcugccgcucuggacaacaccgaccugguguuuggccaguacagagaagccggcgugcugauguaccgggacuacccucuggaacuguucauggcccagugcuacggcaacaucagcgaccucggaaagggcagacagaugccugugcacuacggcugcaaagaacggcacuucgugaccaucagcagcccucuggccacacagauuccucaggcugugggagccgccuacgccgccaaaagagccaacgccaacagaguggucaucugcuacuuuggcgagggcgcugcuucugaaggcgacgcucacgccggcuucaauuucgccgccacucuggagugccccaucaucuuuuucugccggaacaacggcuacgccaucagcacaccuaccagcgagcaguauagaggcgacggaauugccgccagaggaccuggcuauggcaucaugagcaucagaguggacggcaacgacguguucgccguguacaacgccaccaaagaagccaggcggagagccguggcugagaaccagccuuuucugaucgaggccaugaccuaccggaucggccaccacagcaccuccgacgauagcagcgccuacagauccguggacgaagugaacuacugggacaagcaggaucaccccaucuccagacugcggcacuaccugcugucucaaggcuggugggacgaagaacaagagaaggccuggcggaagcagagccggcggaaagugauggaagccuuugagcaggccgagcggaagcccaagccuaauccuaaccugcuguucagcgacguguaccaagagaugccugcucagcugcgcaagcagcaagaaagccuggccagacaccugcagaccuacggcgagcacuacccacuggaccacuucgacaaaugau

SEQ ID NO: 32 - RNA, 1182 nt, Artificial Sequence
  mRNA-E1b
auggcuguuguugcugcugcagcuggauggcugcugagacuuagagcugcuggcgccgaaggccauuggagaagguugccuggugcuggacuggccagaggcuuucugcauccugccgccacaguggaagacgccgcucagagaaggcaggucgcccacuucaccuuccagccugauccugagccuagagaguacggccagacacagaagaugaaccuguuccagagcgugacaagcgcccuggacaacagccuggccaaagauccuaccgccgugaucuucggagaggacguggcauucggcggcguguucagauguacagugggccugagagauaaguacggcaaggaccggguguucaacacaccccugugugaacagggcaucgucggcuucggaaucggaauugcugugacaggcgccaccgccauugccgagauucaguucgccgacuacaucuuccccgccuucgaccagaucgugaacgaggccgccaaguacagauacagaagcggcgaccuguucaacugcggcagccugaccauuagaagcccuuggggcuguguuggacacggcgcccuguaucacucucagagcccugaggccuucuucgcccacuguccuggcaucaagguggucaucccuagaagccccuuucaggccaagggccugcugcuguccugcaucgaggacaagaaccccugcaucuucuucgagcccaagauccuguaccgggcugccgcugaggaagugccuaucgagcccuacaacaucccucugagccaggccgaagugauccaagagggcuccgaugugacacugguggccuggggaacacaggugcacgugaucagagaaguggccagcauggcuaaagagaagcugggcgucagcugugaagugaucgaccugagaacaaucauccccugggacgucgacaccaucugcaaguccgugaucaagaccggcagacugcugaucucucacgaggcuccucugacaggcggcuuugccucugagaucagcuccaccgugcaagaggagugcuuccugaaccuggaagccccaaucagcagagugugcggcuacgacaccccuuuuccacacaucuuugagcccuucuacauccccgacaaguggaagugcuacgacgcccugcggaagaugaucaacuacugauaa